(19) United States
(12) Patent Application Publication
(10) Pub. No.: US 2018/0282324 A1
(43) Pub. Date: Oct. 4, 2018

(54) CRYSTALLINE FORMS OF A PYRROLOPYRIDINE COMPOUND

(71) Applicants: Genentech, Inc., South San Francisco, CA (US); Array Biopharma Inc., Boulder, CO (US)

(72) Inventors: Jeffrey Stults, South San Francisco, CA (US); Christopher M. Lindemann, Fort Collins, CO (US); Keith L. Spencer, Lyons, CO (US); Weidong Liu, Superior, CO (US); Joseph Lubach, Half Moon Bay, CA (US)

(73) Assignees: Genentech, Inc., South San Francisco, CA (US); Array Biopharma Inc., Boulder, CO (US)

(21) Appl. No.: 15/806,650

(22) Filed: Nov. 8, 2017

Related U.S. Application Data
(63) Continuation of application No. 15/054,611, filed on Feb. 26, 2016, now abandoned.
(60) Provisional application No. 62/121,396, filed on Feb. 26, 2015.

(51) Int. Cl. C07D 471/04; A61K 31/437; A61K 45/06

(52) U.S. Cl. CPC C07D 471/04; A61K 45/06; A61K 31/437

(57) ABSTRACT

Disclosed are crystalline forms of (R)—N-(4-(3-aminopiperidin-1-yl)-5-bromo-1H-pyrrolo[2,3-b]pyridin-3-yl)cyclopropanecarboxamide, and salts, solvates, and hydrates thereof, and pharmaceutical compositions, formulations and a process of manufacturing thereof.

### CROSS REFERENCE TO RELATED APPLICATIONS

This non-provisional application filed under 37 CFR § 1.53(b), claims the benefit under 35 USC § 119(e) of U.S. Provisional Application Ser. No. 62/121,396 filed on 26 Feb. 2015, which is incorporated by reference in its entirety.

### FIELD OF THE INVENTION

Disclosed herein are crystalline forms of a pyrrolopyridine compound, and salts solvates, and hydrates thereof, with therapeutic activity, against diseases such as cancer, and processes for making the same.

### BACKGROUND OF INVENTION

CHK1 is a serine/threonine kinase that regulates cell-cycle progression and is a main factor in DNA-damage response within a cell. CHK1 inhibitors have been shown to sensitize tumor cells to a variety of genotoxic agents, such as chemotherapy and radiation. U.S. Pat. No. 8,178,131 discusses a number of inhibitors of CHK1, including the compound (R)—N-(4-(3-aminopiperidin-1-yl)-5-bromo-1H-pyrrolo[2,3-b]pyridin-3-yl)cyclopropanecarboxamide (Compound 1), which is being investigated in clinical trials for the treatment of various cancers.

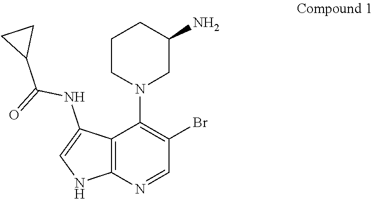 [SMILES: N[C@@H]1CCCN(c2c(Br)cnc3[nH]cc(NC(=O)C4CC4)c23)C1]

What is needed are forms of (R)—N-(4-(3-aminopiperidin-1-yl)-5-bromo-1H-pyrrolo[2,3-b]pyridin-3-yl)cyclopropanecarboxamide and its salts, solvates, and hydrates that have improved pharmaceutical properties.

### SUMMARY OF INVENTION

One aspect includes crystalline forms of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof, and pharmaceutical compositions, formulations and a process of manufacturing thereof.

Another aspect includes use of a crystalline form of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof, or pharmaceutical formulation thereof in therapy.

Another aspect includes a method of treating a cancer comprising administering a crystalline form of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof, or pharmaceutical formulation thereof to a patient in need thereof.

One aspect includes crystalline forms of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof, pharmaceutical compositions, formulations and a process of manufacturing thereof.

Another aspect includes use of a crystalline form of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof, or pharmaceutical formulation thereof in therapy.

Another aspect includes a method of treating a cancer comprising administering a crystalline form of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof, or pharmaceutical formulation thereof to a patient in need thereof.

### DESCRIPTION OF FIGURES

FIG. 1 shows Xray physical characterization of a crystalline form of (R)—N-(4-(3-aminopiperidin-1-yl)-5-bromo-1H-pyrrolo[2,3-b]pyridin-3-yl)cyclopropanecarboxamide acetic acid salt.

FIG. 2 shows Xray physical characterization of a crystalline form of (R)—N-(4-(3-aminopiperidin-1-yl)-5-bromo-1H-pyrrolo[2,3-b]pyridin-3-yl)cyclopropanecarboxamide ethanedisulfonic acid hydrate.

FIG. 3 shows Xray physical characterization of a crystalline form of (R)—N-(4-(3-aminopiperidin-1-yl)-5-bromo-1H-pyrrolo[2,3-b]pyridin-3-yl)cyclopropanecarboxamide fumaric acid hydrate.

FIG. 4 shows Xray physical characterization of a crystalline form (Form A) of the non-solvated free base of (R)—N-(4-(3-aminopiperidin-1-yl)-5-bromo-1H-pyrrolo[2,3-b]pyridin-3-yl)cyclopropanecarboxamide.

FIG. 5 shows differential scanning calorimetry (DSC) and thermogravimetric analysis (TGA) of a crystalline form (Form A) of the non-solvated free base of (R)—N-(4-(3-aminopiperidin-1-yl)-5-bromo-1H-pyrrolo[2,3-b]pyridin-3-yl)cyclopropanecarboxamide.

FIG. 6 shows Xray physical characterization of a crystalline form (Form B) of the non-solvated free base of (R)—N-(4-(3-aminopiperidin-1-yl)-5-bromo-1H-pyrrolo[2,3-b]pyridin-3-yl)cyclopropanecarboxamide.

FIG. 7 shows DSC of a crystalline form (Form B) of the non-solvated free base of (R)—N-(4-(3-aminopiperidin-1-yl)-5-bromo-1H-pyrrolo[2,3-b]pyridin-3-yl)cyclopropanecarboxamide.

FIG. 8 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base cyclopropyl methyl ether solvate.

FIG. 9 shows DSC and TGA of a crystalline form of (R)—N-(4-(3-aminopiperidin-1-yl)-5-bromo-1H-pyrrolo[2,3-b]pyridin-3-yl)cyclopropanecarboxamide free base cyclopropyl methyl ether solvate.

FIG. 10 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base 1,2-dichloroethane solvate.

FIG. 11 shows DSC of a crystalline form of (R)—N-(4-(3-aminopiperidin-1-yl)-5-bromo-1H-pyrrolo[2,3-b]pyridin-3-yl)cyclopropanecarboxamide free base 1,2-dichloroethane solvate.

FIG. 12 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base 2-methyltetrahydrofuran solvate.

FIG. 13 shows DSC of a crystalline form of (R)—N-(4-(3-aminopiperidin-1-yl)-5-bromo-1H-pyrrolo[2,3-b]pyridin-3-yl)cyclopropanecarboxamide free base 2-methyltetrahydrofuran solvate.

FIG. 14 shows the crystal structure of the asymmetric unit of the 2-methyltetrahydrofuran solvate of the free base of (R)—N-(4-(3-aminopiperidin-1-yl)-5-bromo-1H-pyrrolo[2,3-b]pyridin-3-yl)cyclopropanecarboxamide, as determined by single crystal X-ray diffraction (SCXRD).

FIG. 15 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base 1-pentanol solvate.

FIG. 16 shows DSC of a crystalline form of (R)—N-(4-(3-aminopiperidin-1-yl)-5-bromo-1H-pyrrolo[2,3-b]pyridin-3-yl)cyclopropanecarboxamide free base 1-pentanol solvate.

FIG. 17 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base pyridine solvate.

FIG. 18 shows DSC of a crystalline form of (R)—N-(4-(3-aminopiperidin-1-yl)-5-bromo-1H-pyrrolo[2,3-b]pyridin-3-yl)cyclopropanecarboxamide free base pyridine solvate.

FIG. 19 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base 1,4-dioxane solvate.

FIG. 20 shows DSC of a crystalline form of (R)—N-(4-(3-aminopiperidin-1-yl)-5-bromo-1H-pyrrolo[2,3-b]pyridin-3-yl)cyclopropanecarboxamide free base 1,4-dioxane solvate.

FIG. 21 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base 2-butanol solvate.

FIG. 22 shows DSC of a crystalline form of (R)—N-(4-(3-aminopiperidin-1-yl)-5-bromo-1H-pyrrolo[2,3-b]pyridin-3-yl)cyclopropanecarboxamide free base 2-butanol solvate.

FIG. 23 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base anisole solvate.

FIG. 24 shows DSC and TGA of a crystalline form of (R)—N-(4-(3-aminopiperidin-1-yl)-5-bromo-1H-pyrrolo[2,3-b]pyridin-3-yl)cyclopropanecarboxamide free base anisole solvate.

FIG. 25 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base 1-propanol solvate.

FIG. 26 shows the crystal structure of the asymmetric unit of the bis-ethanol solvate of the free base of (R)—N-(4-(3-aminopiperidin-1-yl)-5-bromo-1H-pyrrolo[2,3-b]pyridin-3-yl)cyclopropanecarboxamide, as determined by SCXRD.

FIG. 27 shows an X-ray powder diffraction (XRPD) pattern of a crystalline form of (R)—N-(4-(3-aminopiperidin-1-yl)-5-bromo-1H-pyrrolo[2,3-b]pyridin-3-yl)cyclopropanecarboxamide free base bis-ethanol solvate calculated from the SCXRD data at 100 K.

FIG. 28 shows the crystal structure of the asymmetric unit of the bis-methanol solvate of the free base of (R)—N-(4-(3-aminopiperidin-1-yl)-5-bromo-1H-pyrrolo[2,3-b]pyridin-3-yl)cyclopropanecarboxamide along the crystallographic b axis, as determined by SCXRD.

FIG. 29 shows an XRPD pattern of a crystalline form of (R)—N-(4-(3-aminopiperidin-1-yl)-5-bromo-1H-pyrrolo[2,3-b]pyridin-3-yl)cyclopropanecarboxamide free base bis-methanol solvate calculated from the SCXRD data at 100 K.

FIG. 30 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base methyl tert-butyl ether solvate.

FIG. 31 shows DSC and TGA of a crystalline form of (R)—N-(4-(3-aminopiperidin-1-yl)-5-bromo-1H-pyrrolo[2,3-b]pyridin-3-yl)cyclopropanecarboxamide free base methyl tert-butyl ether solvate.

FIG. 32 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base toluene solvate.

FIG. 33 shows DSC and TGA of a crystalline form of (R)—N-(4-(3-aminopiperidin-1-yl)-5-bromo-1H-pyrrolo[2,3-b]pyridin-3-yl)cyclopropanecarboxamide free base toluene solvate.

FIG. 34 shows Xray physical characterization of a crystalline form of (R)—N-(4-(3-aminopiperidin-1-yl)-5-bromo-1H-pyrrolo[2,3-b]pyridin-3-yl)cyclopropanecarboxamide free base butyronitrile solvate.

FIG. 35 shows DSC and TGA of a crystalline form of (R)—N-(4-(3-aminopiperidin-1-yl)-5-bromo-1H-pyrrolo[2,3-b]pyridin-3-yl)cyclopropanecarboxamide free base butyronitrile solvate.

FIG. 36 shows Xray physical characterization of a crystalline form of (R)—N-(4-(3-aminopiperidin-1-yl)-5-bromo-1H-pyrrolo[2,3-b]pyridin-3-yl)cyclopropanecarboxamide di-methanesulfonic acid salt hydrate.

FIG. 37 shows DSC of a crystalline form of (R)—N-(4-(3-aminopiperidin-1-yl)-5-bromo-1H-pyrrolo[2,3-b]pyridin-3-yl)cyclopropanecarboxamide di-methanesulfonic acid salt hydrate.

FIG. 38 shows TGA of a crystalline form of (R)—N-(4-(3-aminopiperidin-1-yl)-5-bromo-1H-pyrrolo[2,3-b]pyridin-3-yl)cyclopropanecarboxamide di-methanesulfonic acid salt hydrate.

FIG. 39 shows Xray physical characterization of a crystalline form of (R)—N-(4-(3-aminopiperidin-1-yl)-5-bromo-1H-pyrrolo[2,3-b]pyridin-3-yl)cyclopropanecarboxamide di-ethanesulfonic acid salt hydrate.

FIG. 40 shows DSC of a crystalline form of (R)—N-(4-(3-aminopiperidin-1-yl)-5-bromo-1H-pyrrolo[2,3-b]pyridin-3-yl)cyclopropanecarboxamide di-ethanesulfonic acid salt hydrate.

FIG. 41 shows TGA of a crystalline form of (R)—N-(4-(3-aminopiperidin-1-yl)-5-bromo-1H-pyrrolo[2,3-b]pyridin-3-yl)cyclopropanecarboxamide di-ethanesulfonic acid salt hydrate.

FIG. 42 shows Xray physical characterization of a crystalline form of (R)—N-(4-(3-aminopiperidin-1-yl)-5-bromo-1H-pyrrolo[2,3-b]pyridin-3-yl)cyclopropanecarboxamide mono-methanesulfonic acid salt.

FIG. 43 shows DSC and TGA of a crystalline form of (R)—N-(4-(3-aminopiperidin-1-yl)-5-bromo-1H-pyrrolo[2,3-b]pyridin-3-yl)cyclopropanecarboxamide mono-methanesulfonic acid salt.

FIG. 44 shows Xray physical characterization of a crystalline form of (R)—N-(4-(3-aminopiperidin-1-yl)-5-bromo-1H-pyrrolo[2,3-b]pyridin-3-yl)cyclopropanecarboxamide mono-ethanesulfonic acid salt.

FIG. 45 shows DSC and TGA of a crystalline form of (R)—N-(4-(3-aminopiperidin-1-yl)-5-bromo-1H-pyrrolo[2,3-b]pyridin-3-yl)cyclopropanecarboxamide mono-ethanesulfonic acid salt.

FIG. 46 shows Xray physical characterization of a crystalline form of (R)—N-(4-(3-aminopiperidin-1-yl)-5-bromo-1H-pyrrolo[2,3-b]pyridin-3-yl)cyclopropanecarboxamide di-benzenesulfonic acid salt.

FIG. 47 shows Xray physical characterization of a crystalline form of (R)—N-(4-(3-aminopiperidin-1-yl)-5-bromo-1H-pyrrolo[2,3-b]pyridin-3-yl)cyclopropanecarboxamide di-toluenesulfonic acid salt.

FIG. 48 shows DSC of a crystalline form of (R)—N-(4-(3-aminopiperidin-1-yl)-5-bromo-1H-pyrrolo[2,3-b]pyridin-3-yl)cyclopropanecarboxamide di-toluenesulfonic acid salt.

FIG. 49 shows Xray physical characterization of a crystalline form of (R)—N-(4-(3-aminopiperidin-1-yl)-5-bromo-1H-pyrrolo[2,3-b]pyridin-3-yl)cyclopropanecarboxamide di-ethanesulfonic acid salt anhydrate.

FIG. 50 shows DSC of a crystalline form of (R)—N-(4-(3-aminopiperidin-1-yl)-5-bromo-1H-pyrrolo[2,3-b]pyridin-3-yl)cyclopropanecarboxamide di-ethanesulfonic acid salt anhydrate.

FIG. 51 shows TGA of a crystalline form of (R)—N-(4-(3-aminopiperidin-1-yl)-5-bromo-1H-pyrrolo[2,3-b]pyridin-3-yl)cyclopropanecarboxamide di-ethanesulfonic acid salt anhydrate.

FIG. 52 shows DSC and TGA of a crystalline form of (R)—N-(4-(3-aminopiperidin-1-yl)-5-bromo-1H-pyrrolo[2,3-b]pyridin-3-yl)cyclopropanecarboxamide acetic acid salt.

FIG. 53 shows DSC and TGA of a crystalline form of (R)—N-(4-(3-aminopiperidin-1-yl)-5-bromo-1H-pyrrolo[2,3-b]pyridin-3-yl)cyclopropanecarboxamide ethanedisulfonic acid hydrate.

FIG. 54 shows the oral absorption of (R)—N-(4-(3-aminopiperidin-1-yl)-5-bromo-1H-pyrrolo[2,3-b]pyridin-3-yl)cyclopropanecarboxamide free base Form A and (R)—N-(4-(3-aminopiperidin-1-yl)-5-bromo-1H-pyrrolo[2,3-b]pyridin-3-yl)cyclopropanecarboxamide maleic acid salt in PiC formulations, versus (R)—N-(4-(3-aminopiperidin-1-yl)-5-bromo-1H-pyrrolo[2,3-b]pyridin-3-yl)cyclopropanecarboxamide free base in solution, in a preclinical pharmacokinetic study conducted in canines.

### DETAILED DESCRIPTION OF INVENTION

The term “a” as used herein means one or more.

Reference to “about” a value or parameter herein includes (and describes) embodiments that are directed to that value or parameter per se and in one embodiment plus or minus 20% of the given value. For example, description referring to “about X” includes description of “X”.

A “hydrate” refers to an association or complex of one or more water molecules and a compound of the invention.

Compounds of the present invention, unless otherwise indicated, include compounds that differ only in the presence of one or more isotopically enriched atoms. For example, compounds of the present invention, wherein one or more hydrogen atoms are replaced by deuterium or tritium, or one or more carbon atoms are replaced by a ¹³C or ¹⁴C carbon atom, or one or more nitrogen atoms are replaced by a ¹⁵N nitrogen atom, or one or more sulfur atoms are replaced by a ³³S, ³⁴S or ³⁶S sulfur atom, or one or more oxygen atoms are replaced by a ¹⁷O or ¹⁸O oxygen atom are within the scope of this invention.

It has been unexpectedly discovered that isolating (R)—N-(4-(3-aminopiperidin-1-yl)-5-bromo-1H-pyrrolo[2,3-b]pyridin-3-yl)cyclopropanecarboxamide, and salts, solvates, and hydrates thereof, from particular solvents produces different physical forms of the compound, and that the different forms have different pharmaceutical properties. It has been found that certain forms have improved properties useful for formulating the compound into stable drug forms for treating diseases such as cancer.

Therefore, one aspect includes crystalline forms of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof, pharmaceutical compositions, formulations and a process of manufacturing thereof.

Another aspect includes crystalline form of an acetic acid salt of (R)—N-(4-(3-aminopiperidin-1-yl)-5-bromo-1H-pyrrolo[2,3-b]pyridin-3-yl)cyclopropanecarboxamide, pharmaceutical compositions, formulations and a process of manufacturing thereof.

Another aspect includes crystalline form of an ethanedisulfonic acid salt of (R)—N-(4-(3-aminopiperidin-1-yl)-5-bromo-1H-pyrrolo[2,3-b]pyridin-3-yl)cyclopropanecarboxamide or hydrates thereof, pharmaceutical compositions, formulations and a process of manufacturing thereof.

Another aspect includes crystalline form of a fumaric acid salt of (R)—N-(4-(3-aminopiperidin-1-yl)-5-bromo-1H-pyrrolo[2,3-b]pyridin-3-yl)cyclopropanecarboxamide or hydrates thereof, pharmaceutical compositions, formulations and a process of manufacturing thereof.

Another aspect includes a tablet formulation, comprising a crystalline form of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof and a process of manufacturing thereof.

Another aspect includes crystalline forms of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt; (R)—N-(4-(3-aminopiperidin-1-yl)-5-bromo-1H-pyrrolo[2,3-b]pyridin-3-yl)cyclopropanecarboxamide fumaric acid salt; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide di-methanesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and pharmaceutically acceptable solvates and hydrates thereof; and pharmaceutical compositions, formulations and a process of manufacturing thereof.

Another aspect includes crystalline forms of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt hydrate; (R)—N-(4-(3-aminopiperidin-1-yl)-5-bromo-1H-pyrrolo[2,3-b]pyridin-3-yl)cyclopropanecarboxamide fumaric acid salt hydrate; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide cyclopropyl methyl ether solvate; (R)—N-(4-(3-aminopiperidin-1-yl)-5-bromo-1H-pyrrolo[2,3-b]pyridin-3-yl)cyclopropanecarboxamide 1,2-dichloroethane solvate; (R)—N-(4-(3-aminopiperidin-1-yl)-5-bromo-1H-pyrrolo[2,3-b]pyridin-3-yl)cyclopropanecarboxamide 2-methyltetrahydrofuran solvate; (R)—N-(4-(3-aminopiperidin-1-yl)-5-bromo-1H-pyrrolo[2,3-b]pyridin-3-yl)cyclopropanecarboxamide 1-pentanol solvate; (R)—N-(4-(3-aminopiperidin-1-yl)-5-bromo-1H-pyrrolo[2,3-b]pyridin-3-yl)cyclopropanecarboxamide pyridine solvate; (R)—N-(4-(3-aminopiperidin-1-yl)-5-bromo-1H-pyrrolo[2,3-b]pyridin-3-yl)cyclopropanecarboxamide 1,4-dioxane solvate; (R)—N-(4-(3-aminopiperidin-1-yl)-5-bromo-1H-pyrrolo[2,3-b]pyridin-3-yl)cyclopropanecarboxamide 2-butanol solvate; (R)—N-(4-(3-aminopiperidin-1-yl)-5-bromo-1H-pyrrolo[2,3-b]pyridin-3-yl)cyclopropanecarboxamide anisole solvate; (R)—N-(4-(3-aminopiperidin-1-yl)-5-bromo-1H-pyrrolo[2,3-b]pyridin-3-yl)cyclopropanecarboxamide 1-propanol solvate; (R)—N-(4-(3-aminopiperidin-1-yl)-5-bromo-1H-pyrrolo[2,3-b]pyridin-3-yl)cyclopropanecarboxamide bis-ethanol solvate; (R)—N-(4-(3-aminopiperidin-1-yl)-5-bromo-1H-pyrrolo[2,3-b]pyridin-3-yl)cyclopropanecarboxamide bis-methanol solvate; (R)—N-(4-(3-aminopiperidin-1-yl)-5-bromo-1H-pyrrolo[2,3-b]pyridin-3-yl)cyclopropanecarboxamide methyl tert-butyl ether solvate; (R)—N-(4-(3-aminopiperidin-1-yl)-5-bromo-1H-pyrrolo[2,3-b]pyridin-3-yl)cyclopropanecarboxamide toluene solvate; (R)—N-(4-(3-aminopiperidin-1-yl)-5-bromo-1H-pyrrolo[2,3-b]pyridin-3-yl)cyclopropanecarboxamide butyronitrile solvate; (R)—N-(4-(3-aminopiperidin-1-yl)-5-bromo-1H-pyrrolo[2,3-b]pyridin-3-yl)cyclopropanecarboxamide di-methanesulfonic acid salt hydrate; (R)—N-(4-(3-aminopiperidin-1-yl)-5-bromo-1H-pyrrolo[2,3-b]pyridin-3-yl)cyclopropanecarboxamide di-ethanesulfonic acid salt hydrate; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and pharmaceutical compositions, formulations and a process of manufacturing thereof.

Another aspect includes a crystalline form of (R)—N-(4-(3-aminopiperidin-1-yl)-5-bromo-1H-pyrrolo[2,3-b]pyridin-3-yl)cyclopropanecarboxamide; and pharmaceutical compositions, formulations and a process of manufacturing thereof. In some embodiments, the crystalline form is Form A. In some embodiments, the crystalline form is Form B.

Another aspect includes a composition comprising a crystalline form of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof, and a solvent selected from: cyclopropyl methyl ether, 1-pentanol, 2-butanol, anisole, 1-propanol, ethanol, methanol, and methyl tert-butyl ether.

Another aspect includes a tablet formulation, comprising a crystalline form of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt; (R)—N-(4-(3-aminopiperidin-1-yl)-5-bromo-1H-pyrrolo[2,3-b]pyridin-3-yl)cyclopropanecarboxamide fumaric acid salt; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide di-methanesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and pharmaceutically acceptable solvates and hydrates thereof; and a process of manufacturing thereof.

Another aspect includes a tablet formulation, comprising a crystalline form of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt hydrate; (R)—N-(4-(3-aminopiperidin-1-yl)-5-bromo-1H-pyrrolo[2,3-b]pyridin-3-yl)cyclopropanecarboxamide fumaric acid salt hydrate; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide cyclopropyl methyl ether solvate; (R)—N-(4-(3-aminopiperidin-1-yl)-5-bromo-1H-pyrrolo[2,3-b]pyridin-3-yl)cyclopropanecarboxamide 1,2-dichloroethane solvate; (R)—N-(4-(3-aminopiperidin-1-yl)-5-bromo-1H-pyrrolo[2,3-b]pyridin-3-yl)cyclopropanecarboxamide 2-methyltetrahydrofuran solvate; (R)—N-(4-(3-aminopiperidin-1-yl)-5-bromo-1H-pyrrolo[2,3-b]pyridin-3-yl)cyclopropanecarboxamide 1-pentanol solvate; (R)—N-(4-(3-aminopiperidin-1-yl)-5-bromo-1H-pyrrolo[2,3-b]pyridin-3-yl)cyclopropanecarboxamide pyridine solvate; (R)—N-(4-(3-aminopiperidin-1-yl)-5-bromo-1H-pyrrolo[2,3-b]pyridin-3-yl)cyclopropanecarboxamide 1,4-dioxane solvate; (R)—N-(4-(3-aminopiperidin-1-yl)-5-bromo-1H-pyrrolo[2,3-b]pyridin-3-yl)cyclopropanecarboxamide 2-butanol solvate; (R)—N-(4-(3-aminopiperidin-1-yl)-5-bromo-1H-pyrrolo[2,3-b]pyridin-3-yl)cyclopropanecarboxamide anisole solvate; (R)—N-(4-(3-aminopiperidin-1-yl)-5-bromo-1H-pyrrolo[2,3-b]pyridin-3-yl)cyclopropanecarboxamide 1-propanol solvate; (R)—N-(4-(3-aminopiperidin-1-yl)-5-bromo-1H-pyrrolo[2,3-b]pyridin-3-yl)cyclopropanecarboxamide bis-ethanol solvate; (R)—N-(4-(3-aminopiperidin-1-yl)-5-bromo-1H-pyrrolo[2,3-b]pyridin-3-yl)cyclopropanecarboxamide bis-methanol solvate; (R)—N-(4-(3-aminopiperidin-1-yl)-5-bromo-1H-pyrrolo[2,3-b]pyridin-3-yl)cyclopropanecarboxamide methyl tert-butyl ether solvate; (R)—N-(4-(3-aminopiperidin-1-yl)-5-bromo-1H-pyrrolo[2,3-b]pyridin-3-yl)cyclopropanecarboxamide toluene solvate; (R)—N-(4-(3-aminopiperidin-1-yl)-5-bromo-1H-pyrrolo[2,3-b]pyridin-3-yl)cyclopropanecarboxamide butyronitrile solvate; (R)—N-(4-(3-aminopiperidin-1-yl)-5-bromo-1H-pyrrolo[2,3-b]pyridin-3-yl)cyclopropanecarboxamide di-methanesulfonic acid salt hydrate; (R)—N-(4-(3-aminopiperidin-1-yl)-5-bromo-1H-pyrrolo[2,3-b]pyridin-3-yl)cyclopropanecarboxamide di-ethanesulfonic acid salt hydrate; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and a process of manufacturing thereof.

Another aspect includes a tablet formulation, comprising a crystalline form of (R)—N-(4-(3-aminopiperidin-1-yl)-5-bromo-1H-pyrrolo[2,3-b]pyridin-3-yl)cyclopropanecarboxamide; and a process of manufacturing thereof. In some embodiments, the crystalline form is Form A. In some embodiments, the crystalline form is Form B.

Methods of Treatment with Crystalline Form of the Present Invention

The compounds described herein can be used as therapeutic agents for treating diseases.

In one embodiment, compounds of the present invention can be used for the treatment of hyperproliferative disorders, including cancers of the following categories: (1) Cardiac: sarcoma (angiosarcoma, fibrosarcoma, rhabdomyosarcoma, liposarcoma), myxoma, rhabdomyoma, fibroma, lipoma and teratoma; (2) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung, small cell lung; (3) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); (4) Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); (5) Liver: hepatoma (hepatocellular carcinoma), cholangiocarcinoma, hepatoblastoma, angiosarcoma, hepatocellular adenoma, hemangioma; (6) Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; (7) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme. oligodendroglioma, schwannoma, retinoblastoma, congenital tumors), spinal cord neurofibroma, meningioma, glioma, sarcoma); (8) Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); (9) Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; (10) Skin: advanced melanoma, malignant melanoma, basal cell carcinoma, squamous cell carcinoma, Karposi's sarcoma, moles dysplastic nevi, lipoma, angioma, dermatofibroma, keloids, psoriasis; (11) Adrenal glands: neuroblastoma; (12) Breast: metastatic breast; breast adenocarcinoma; (13) Colon; (14) Oral cavity; (15) Hairy cell leukemia; (16) Head and neck; (17) and others including refractory metastatic disease; Kaposi's sarcoma; Bannayan-Zonana syndrome; and Cowden disease or Lhermitte-Duclos disease, among other kinds of hyperproliferative disorders. In one example, the disease is triple negative breast cancer.

Accordingly, another aspect of this invention provides a method of treating diseases or medical conditions in a mammal, comprising administering to said mammal one or more crystalline forms of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof in an amount effective to treat or prevent said disorder.

Accordingly, another aspect of this invention provides a method of treating diseases or medical conditions in a mammal, comprising administering to said mammal one or more crystalline forms of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof; in an amount effective to treat or prevent said disorder.

Accordingly, another aspect of this invention provides a method of treating diseases or medical conditions in a mammal, comprising administering to said mammal one or more crystalline forms of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt; (R)—N-(4-(3-aminopiperidin-1-yl)-5-bromo-1H-pyrrolo[2,3-b]pyridin-3-yl)cyclopropanecarboxamide fumaric acid salt; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide di-methanesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and pharmaceutically acceptable solvates and hydrates thereof; in an amount effective to treat or prevent said disorder.

Accordingly, another aspect of this invention provides a method of treating diseases or medical conditions in a mammal, comprising administering to said mammal one or more crystalline forms of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt hydrate; (R)—N-(4-(3-aminopiperidin-1-yl)-5-bromo-1H-pyrrolo[2,3-b]pyridin-3-yl)cyclopropanecarboxamide fumaric acid salt hydrate; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide cyclopropyl methyl ether solvate; (R)—N-(4-(3-aminopiperidin-1-yl)-5-bromo-1H-pyrrolo[2,3-b]pyridin-3-yl)cyclopropanecarboxamide 1,2-dichloroethane solvate; (R)—N-(4-(3-aminopiperidin-1-yl)-5-bromo-1H-pyrrolo[2,3-b]pyridin-3-yl)cyclopropanecarboxamide 2-methyltetrahydrofuran solvate; (R)—N-(4-(3-aminopiperidin-1-yl)-5-bromo-1H-pyrrolo[2,3-b]pyridin-3-yl)cyclopropanecarboxamide 1-pentanol solvate; (R)—N-(4-(3-aminopiperidin-1-yl)-5-bromo-1H-pyrrolo[2,3-b]pyridin-3-yl)cyclopropanecarboxamide pyridine solvate; (R)—N-(4-(3-aminopiperidin-1-yl)-5-bromo-1H-pyrrolo[2,3-b]pyridin-3-yl)cyclopropanecarboxamide 1,4-dioxane solvate; (R)—N-(4-(3-aminopiperidin-1-yl)-5-bromo-1H-pyrrolo[2,3-b]pyridin-3-yl)cyclopropanecarboxamide 2-butanol solvate; (R)—N-(4-(3-aminopiperidin-1-yl)-5-bromo-1H-pyrrolo[2,3-b]pyridin-3-yl)cyclopropanecarboxamide anisole solvate; (R)—N-(4-(3-aminopiperidin-1-yl)-5-bromo-1H-pyrrolo[2,3-b]pyridin-3-yl)cyclopropanecarboxamide 1-propanol solvate; (R)—N-(4-(3-aminopiperidin-1-yl)-5-bromo-1H-pyrrolo[2,3-b]pyridin-3-yl)cyclopropanecarboxamide bis-ethanol solvate; (R)—N-(4-(3-aminopiperidin-1-yl)-5-bromo-1H-pyrrolo[2,3-b]pyridin-3-yl)cyclopropanecarboxamide bis-methanol solvate; (R)—N-(4-(3-aminopiperidin-1-yl)-5-bromo-1H-pyrrolo[2,3-b]pyridin-3-yl)cyclopropanecarboxamide methyl tert-butyl ether solvate; (R)—N-(4-(3-aminopiperidin-1-yl)-5-bromo-1H-pyrrolo[2,3-b]pyridin-3-yl)cyclopropanecarboxamide toluene solvate; (R)—N-(4-(3-aminopiperidin-1-yl)-5-bromo-1H-pyrrolo[2,3-b]pyridin-3-yl)cyclopropanecarboxamide butyronitrile solvate; (R)—N-(4-(3-aminopiperidin-1-yl)-5-bromo-1H-pyrrolo[2,3-b]pyridin-3-yl)cyclopropanecarboxamide di-methanesulfonic acid salt hydrate; (R)—N-(4-(3-aminopiperidin-1-yl)-5-bromo-1H-pyrrolo[2,3-b]pyridin-3-yl)cyclopropanecarboxamide di-ethanesulfonic acid salt hydrate; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; in an amount effective to treat or prevent said disorder.

Accordingly, another aspect of this invention provides a method of treating diseases or medical conditions in a mammal, comprising administering to said mammal a crystalline form of (R)—N-(4-(3-aminopiperidin-1-yl)-5-bromo-1H-pyrrolo[2,3-b]pyridin-3-yl)cyclopropanecarboxamide; in an amount effective to treat or prevent said disorder. In some embodiments, the crystalline form is Form A. In some embodiments, the crystalline form is Form B.

The phrase “effective amount” means an amount of compound that, when administered to a mammal in need of such treatment, is sufficient to (i) attenuate, ameliorate, or eliminate one or more symptoms of the particular disease, condition, or disorder, or (iii) prevent or delay the onset of one or more symptoms of the particular disease, condition, or disorder described herein. In the case of cancer, an effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The amount of a compound of the present invention that will correspond to such an effective amount will vary depending upon factors such as the particular compound, disease condition and its severity, the identity (e.g., weight) of the mammal in need of treatment, but can nevertheless be routinely determined by one skilled in the art.

The terms “treat” and “treatment” refer to therapeutic treatment, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as having been diagnosed as having it by a licensed medical doctor.

As used herein, the term “mammal” refers to a warm-blooded animal that has or is at risk of developing a disease described herein and includes, but is not limited to, guinea pigs, dogs, cats, rats, mice, hamsters, and primates, including humans.

This invention also provides crystalline forms of the present invention for use in the treatment of CHK1 protein kinase-mediated conditions.

An additional aspect of the invention is the use of a crystalline form of the present invention in the preparation of a medicament for therapy, such as for the treatment or prevention of CHK1 protein kinase-mediated conditions.

One aspect of the present invention relates to a method of treating a disease or disorder modulated by CHK1, comprising administering a crystalline form of the present invention, or pharmaceutical formulation thereof, to a patient in need thereof. In some embodiments, the disease is cancer. In some embodiments, the cancer is selected from: leukemia, pancreatic cancer, breast cancer, colon cancer, rectal cancer, colorectal cancer, a refractory solid tumor, and lymphoma.

Another aspect of the present invention relates to a use of a crystalline form of the present invention, in the manufacture of a medicament for treating a disease or disorder modulated by CHK1. In some embodiments, the disease is cancer. In some embodiments, the cancer is selected from: leukemia, pancreatic cancer, breast cancer, colon cancer, rectal cancer, colorectal cancer, a refractory solid tumor, and lymphoma.

Another aspect of the present invention relates to a crystalline form of the present invention, or pharmaceutical formulation thereof, for use in a method of treatment of the human or animal body by therapy.

Another aspect of the present invention relates to a crystalline form of the present invention, or pharmaceutical formulation thereof, for use in a method of treating a disease or disorder modulated by CHK1. In some embodiments, the disease is cancer. In some embodiments, the cancer is selected from: leukemia, pancreatic cancer, breast cancer, colon cancer, rectal cancer, colorectal cancer, a refractory solid tumor, and lymphoma.

### Combination Therapy

The compounds of the present invention can be used in combination with one or more additional drugs such as described below. The dose of the second drug can be appropriately selected based on a clinically employed dose. The proportion of the compound of the present invention and the second drug can be appropriately determined according to the administration subject, the administration route, the target disease, the clinical condition, the combination, and other factors. In cases where the administration subject is a human, for instance, the second drug may be used in an amount of 0.01 to 100 parts by weight per part by weight of the compound of the present invention.

The second compound of the pharmaceutical combination formulation or dosing regimen preferably has complementary activities to the compound of this invention such that they do not adversely affect each other. Such drugs are suitably present in combination in amounts that are effective for the purpose intended. Accordingly, another aspect of the present invention provides a composition comprising a compound of this invention in combination with a second drug, such as described herein.

A compound of this invention and the additional pharmaceutically active drug(s) may be administered together in a unitary pharmaceutical composition or separately and, when administered separately this may occur simultaneously or sequentially in any order. Such sequential administration may be close in time or remote in time. The amounts of the compound of this invention and the second drug(s) and the relative timings of administration will be selected in order to achieve the desired combined therapeutic effect.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

A “chemotherapeutic agent” is a chemical compound useful in the treatment of cancer, regardless of mechanism of action. Chemotherapeutic agents include compounds used in “targeted therapy” and conventional chemotherapy.

Examples of chemotherapeutic agents include gemcitabine, Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNEO, Wyeth), Lapatinib (TYKERB®, GSK572016, Glaxo Smith Kline), Lonafarnib (SCH 66336), Sorafenib (BAY43-9006, Bayer Labs), Irinotecan (CAMPTOSAR®, Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chlorambucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASINO (exemestane; Pfizer), formestanie, fadrozole, RIVISORO (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTINO, LEUVECTINO, and VAXID®; PROLEUKINO rIL-2; a topoisomerase 1 inhibitor such as LURTOTECAN®; ABARELIX® rmRH; (ix) anti-angiogenic agents such as bevacizumab (AVASTINO, Genentech); and (x) pharmaceutically acceptable salts, acids and derivatives of any of the above.

Also included in the definition of “chemotherapeutic agent” are therapeutic antibodies such as alemtuzumab (Campath), bevacizumab (AVASTINO, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTINO, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the PI3K inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Accordingly one aspect of the present invention relates to a pharmaceutical formulation comprising a crystalline form of the present invention and a DNA damaging agent. In some embodiments, the DNA damaging agent is selected from: gemcitabine, irinotecan, temozolomide, capecitabine, camptothecin, cisplatin, ara-C, and 5-FU. In some embodiments, the formulation further comprises an excipient. In some embodiments, the formulation is a tablet for oral delivery.

Another aspect of the present invention relates to a method of treating a disease or disorder modulated by CHK1, comprising administering a crystalline form of the present invention, or pharmaceutical formulation thereof, to a patient in need thereof, wherein a DNA damaging agent is also administered. In some embodiments, the disease is cancer. In some embodiments, the cancer is selected from: leukemia, pancreatic cancer, breast cancer, colon cancer, rectal cancer, colorectal cancer, a refractory solid tumor, and lymphoma. In some embodiments, the DNA damaging agent is selected from: gemcitabine, irinotecan, temozolomide, capecitabine, camptothecin, cisplatin, ara-C, and 5-FU.

Another aspect of the present invention relates to a use of a crystalline form of the present invention, in the manufacture of a medicament for treating a disease or disorder modulated by CHK1, wherein the medicament further comprises a DNA damaging agent. In some embodiments, the disease is cancer. In some embodiments, the cancer is selected from: leukemia, pancreatic cancer, breast cancer, colon cancer, rectal cancer, colorectal cancer, a refractory solid tumor, and lymphoma. In some embodiments, the DNA damaging agent is selected from: gemcitabine, irinotecan, temozolomide, capecitabine, camptothecin, cisplatin, ara-C, and 5-FU.

Another aspect of the present invention relates to a crystalline form of the present invention, or pharmaceutical formulation thereof, for use in a method of treating a disease or disorder modulated by CHK1, in combination with a DNA damaging agent. In some embodiments, the disease is cancer. In some embodiments, the cancer is selected from: leukemia, pancreatic cancer, breast cancer, colon cancer, rectal cancer, colorectal cancer, a refractory solid tumor, and lymphoma. In some embodiments, the DNA damaging agent is selected from: gemcitabine, irinotecan, temozolomide, capecitabine, camptothecin, cisplatin, ara-C, and 5-FU.

### Routes of Administration

The compounds of the invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

### Pharmaceutical Formulations

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition that comprises a compound of this invention. In certain embodiments, the pharmaceutical composition comprises a crystalline form of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof in association with a pharmaceutically acceptable diluent or carrier.

In certain embodiments, the pharmaceutical composition comprises a crystalline form of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof; in association with a pharmaceutically acceptable diluent or carrier.

In certain embodiments, the pharmaceutical composition comprises a composition comprising a crystalline form of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof, and a solvent selected from: cyclopropyl methyl ether, 1-pentanol, 2-butanol, anisole, 1-propanol, ethanol, methanol, and methyl tert-butyl ether; in association with a pharmaceutically acceptable diluent or carrier.

In certain embodiments, the pharmaceutical composition comprises a crystalline form of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt; (R)—N-(4-(3-aminopiperidin-1-yl)-5-bromo-1H-pyrrolo[2,3-b]pyridin-3-yl)cyclopropanecarboxamide fumaric acid salt; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide di-methanesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and pharmaceutically acceptable solvates and hydrates thereof; in association with a pharmaceutically acceptable diluent or carrier.

In certain embodiments, the pharmaceutical composition comprises a crystalline form of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt hydrate; (R)—N-(4-(3-aminopiperidin-1-yl)-5-bromo-1H-pyrrolo[2,3-b]pyridin-3-yl)cyclopropanecarboxamide fumaric acid salt hydrate; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide cyclopropyl methyl ether solvate; (R)—N-(4-(3-aminopiperidin-1-yl)-5-bromo-1H-pyrrolo[2,3-b]pyridin-3-yl)cyclopropanecarboxamide 1,2-dichloroethane solvate; (R)—N-(4-(3-aminopiperidin-1-yl)-5-bromo-1H-pyrrolo[2,3-b]pyridin-3-yl)cyclopropanecarboxamide 2-methyltetrahydrofuran solvate; (R)—N-(4-(3-aminopiperidin-1-yl)-5-bromo-1H-pyrrolo[2,3-b]pyridin-3-yl)cyclopropanecarboxamide 1-pentanol solvate; (R)—N-(4-(3-aminopiperidin-1-yl)-5-bromo-1H-pyrrolo[2,3-b]pyridin-3-yl)cyclopropanecarboxamide pyridine solvate; (R)—N-(4-(3-aminopiperidin-1-yl)-5-bromo-1H-pyrrolo[2,3-b]pyridin-3-yl)cyclopropanecarboxamide 1,4-dioxane solvate; (R)—N-(4-(3-aminopiperidin-1-yl)-5-bromo-1H-pyrrolo[2,3-b]pyridin-3-yl)cyclopropanecarboxamide 2-butanol solvate; (R)—N-(4-(3-aminopiperidin-1-yl)-5-bromo-1H-pyrrolo[2,3-b]pyridin-3-yl)cyclopropanecarboxamide anisole solvate; (R)—N-(4-(3-aminopiperidin-1-yl)-5-bromo-1H-pyrrolo[2,3-b]pyridin-3-yl)cyclopropanecarboxamide 1-propanol solvate; (R)—N-(4-(3-aminopiperidin-1-yl)-5-bromo-1H-pyrrolo[2,3-b]pyridin-3-yl)cyclopropanecarboxamide bis-ethanol solvate; (R)—N-(4-(3-aminopiperidin-1-yl)-5-bromo-1H-pyrrolo[2,3-b]pyridin-3-yl)cyclopropanecarboxamide bis-methanol solvate; (R)—N-(4-(3-aminopiperidin-1-yl)-5-bromo-1H-pyrrolo[2,3-b]pyridin-3-yl)cyclopropanecarboxamide methyl tert-butyl ether solvate; (R)—N-(4-(3-aminopiperidin-1-yl)-5-bromo-1H-pyrrolo[2,3-b]pyridin-3-yl)cyclopropanecarboxamide toluene solvate; (R)—N-(4-(3-aminopiperidin-1-yl)-5-bromo-1H-pyrrolo[2,3-b]pyridin-3-yl)cyclopropanecarboxamide butyronitrile solvate; (R)—N-(4-(3-aminopiperidin-1-yl)-5-bromo-1H-pyrrolo[2,3-b]pyridin-3-yl)cyclopropanecarboxamide di-methanesulfonic acid salt hydrate; (R)—N-(4-(3-aminopiperidin-1-yl)-5-bromo-1H-pyrrolo[2,3-b]pyridin-3-yl)cyclopropanecarboxamide di-ethanesulfonic acid salt hydrate; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; in association with a pharmaceutically acceptable diluent or carrier.

In certain embodiments, the pharmaceutical composition comprises a crystalline form of (R)—N-(4-(3-aminopiperidin-1-yl)-5-bromo-1H-pyrrolo[2,3-b]pyridin-3-yl)cyclopropanecarboxamide; in association with a pharmaceutically acceptable diluent or carrier. In some embodiments, the crystalline form is Form A. In some embodiments, the crystalline form is Form B.

The pharmaceutical compositions of the invention are formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutically effective amount of the compound to be administered will be governed by such considerations, and is the minimum amount necessary to prevent, ameliorate, or treat the disorder. The compound of the present invention is typically formulated into pharmaceutical dosage forms to provide an easily controllable dosage of the drug and to enable patient compliance with the prescribed regimen.

The composition for use herein is preferably sterile. In particular, formulations to be used for in vivo administration must be sterile. Such sterilization is readily accomplished, for example, by filtration through sterile filtration membranes. The compound ordinarily can be stored as a solid composition, a lyophilized formulation or as an aqueous solution.

Pharmaceutical formulations of the compounds of the present invention may be prepared for various routes and types of administration. For example, a compound of this invention having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, a milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. The formulations may be prepared using conventional dissolution and mixing procedures.

The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The formulations may also include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Sustained-release preparations of compounds of this invention may be prepared. Suitable examples of sustained-release preparations include semipermeable matrices of solid hydrophobic polymers containing a crystalline form of the present invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The compositions of the invention may also be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder).

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical composition (or formulation) for application may be packaged in a variety of ways depending upon the method used for administering the drug. For example, an article for distribution can include a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The formulations may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The amount of a compound of this invention that is combined with one or more excipients to produce a single dosage form will necessarily vary depending upon the subject treated, the severity of the disorder or condition, the rate of administration, the disposition of the compound and the discretion of the prescribing physician. In one embodiment, a suitable amount of a compound of this invention is administered to a mammal in need thereof. Administration in one embodiment occurs in an amount between about 0.001 mg/kg of body weight to about 60 mg/kg of body weight per day. In another embodiment, administration occurs in an amount between 0.5 mg/kg of body weight to about 40 mg/kg of body weight per day. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

### Articles of Manufacture

In another embodiment of the invention, an article of manufacture, or “kit”, containing materials useful for the treatment of the disorders described above is provided. In one embodiment, the kit comprises a container comprising a compound of this invention. Suitable containers include, for example, bottles, vials, syringes, blister pack, etc. The container may be formed from a variety of materials such as glass or plastic. The container may hold a compound of this invention or a formulation thereof which is effective for treating the condition and may have a sterile access port (for example, the container may be an intravenous solution bag or a vial having a stopper pierceable by a hypodermic injection needle).

The kit may further comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. In one embodiment, the label or package inserts indicates that the composition comprising a compound of this invention can be used to treat a disorder such as cancer. The label or package insert may also indicate that the composition can be used to treat other disorders.

In certain embodiments, the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to another embodiment, a kit may comprise (a) a first container with a compound of this invention contained therein; and (b) a second container with a second pharmaceutical formulation contained therein, wherein the second pharmaceutical formulation comprises a second compound useful for treating a disorder such as cancer. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of this invention and, if present, the second pharmaceutical formulation. For example, if the kit comprises a first composition comprising a compound of this invention and a second pharmaceutical formulation, the kit may further comprise directions for the simultaneous, sequential or separate administration of the first and second pharmaceutical compositions to a patient in need thereof.

In certain other embodiments wherein the kit comprises a composition of this invention and a second therapeutic agent, the kit may comprise a container for containing the separate compositions such as a divided bottle or a divided foil packet, however, the separate compositions may also be contained within a single, undivided container. In certain embodiments, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Accordingly, a further aspect of this invention provides a kit for treating a disorder, wherein said kit comprises a) a first pharmaceutical composition comprising a crystalline form of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof; and b) instructions for use.

Accordingly, a further aspect of this invention provides a kit for treating a disorder, wherein said kit comprises a) a first pharmaceutical composition comprising a crystalline form of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof; and b) instructions for use.

Accordingly, a further aspect of this invention provides a kit for treating a disorder, wherein said kit comprises a) a first pharmaceutical composition comprising a crystalline form of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt; (R)—N-(4-(3-aminopiperidin-1-yl)-5-bromo-1H-pyrrolo[2,3-b]pyridin-3-yl)cyclopropanecarboxamide fumaric acid salt; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide di-methanesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and pharmaceutically acceptable solvates and hydrates thereof; and b) instructions for use.

Accordingly, a further aspect of this invention provides a kit for treating a disorder, wherein said kit comprises a) a first pharmaceutical composition comprising a crystalline form of a compound selected from: (R)—N-(4-(3-aminopiperidin-1-yl)-5-bromo-1H-pyrrolo[2,3-b]pyridin-3-yl)cyclopropanecarboxamide acetic acid salt; (R)—N-(4-(3-aminopiperidin-1-yl)-5-bromo-1H-pyrrolo[2,3-b]pyridin-3-yl)cyclopropanecarboxamide ethanedisulfonic acid salt hydrate; (R)—N-(4-(3-aminopiperidin-1-yl)-5-bromo-1H-pyrrolo[2,3-b]pyridin-3-yl)cyclopropanecarboxamide fumaric acid salt hydrate; (R)—N-(4-(3-aminopiperidin-1-yl)-5-bromo-1H-pyrrolo[2,3-b]pyridin-3-yl)cyclopropanecarboxamide; (R)—N-(4-(3-aminopiperidin-1-yl)-5-bromo-1H-pyrrolo[2,3-b]pyridin-3-yl)cyclopropanecarboxamide cyclopropyl methyl ether solvate; (R)—N-(4-(3-aminopiperidin-1-yl)-5-bromo-1H-pyrrolo[2,3-b]pyridin-3-yl)cyclopropanecarboxamide 1,2-dichloroethane solvate; (R)—N-(4-(3-aminopiperidin-1-yl)-5-bromo-1H-pyrrolo[2,3-b]pyridin-3-yl)cyclopropanecarboxamide 2-methyltetrahydrofuran solvate; (R)—N-(4-(3-aminopiperidin-1-yl)-5-bromo-1H-pyrrolo[2,3-b]pyridin-3-yl)cyclopropanecarboxamide 1-pentanol solvate; (R)—N-(4-(3-aminopiperidin-1-yl)-5-bromo-1H-pyrrolo[2,3-b]pyridin-3-yl)cyclopropanecarboxamide pyridine solvate; (R)—N-(4-(3-aminopiperidin-1-yl)-5-bromo-1H-pyrrolo[2,3-b]pyridin-3-yl)cyclopropanecarboxamide 1,4-dioxane solvate; (R)—N-(4-(3-aminopiperidin-1-yl)-5-bromo-1H-pyrrolo[2,3-b]pyridin-3-yl)cyclopropanecarboxamide 2-butanol solvate; (R)—N-(4-(3-aminopiperidin-1-yl)-5-bromo-1H-pyrrolo[2,3-b]pyridin-3-yl)cyclopropanecarboxamide anisole solvate; (R)—N-(4-(3-aminopiperidin-1-yl)-5-bromo-1H-pyrrolo[2,3-b]pyridin-3-yl)cyclopropanecarboxamide 1-propanol solvate; (R)—N-(4-(3-aminopiperidin-1-yl)-5-bromo-1H-pyrrolo[2,3-b]pyridin-3-yl)cyclopropanecarboxamide bis-ethanol solvate; (R)—N-(4-(3-aminopiperidin-1-yl)-5-bromo-1H-pyrrolo[2,3-b]pyridin-3-yl)cyclopropanecarboxamide bis-methanol solvate; (R)—N-(4-(3-aminopiperidin-1-yl)-5-bromo-1H-pyrrolo[2,3-b]pyridin-3-yl)cyclopropanecarboxamide methyl tert-butyl ether solvate; (R)—N-(4-(3-aminopiperidin-1-yl)-5-bromo-1H-pyrrolo[2,3-b]pyridin-3-yl)cyclopropanecarboxamide toluene solvate; (R)—N-(4-(3-aminopiperidin-1-yl)-5-bromo-1H-pyrrolo[2,3-b]pyridin-3-yl)cyclopropanecarboxamide butyronitrile solvate; (R)—N-(4-(3-aminopiperidin-1-yl)-5-bromo-1H-pyrrolo[2,3-b]pyridin-3-yl)cyclopropanecarboxamide di-methanesulfonic acid salt hydrate; (R)—N-(4-(3-aminopiperidin-1-yl)-5-bromo-1H-pyrrolo[2,3-b]pyridin-3-yl)cyclopropanecarboxamide di-ethanesulfonic acid salt hydrate; (R)—N-(4-(3-aminopiperidin-1-yl)-5-bromo-1H-pyrrolo[2,3-b]pyridin-3-yl)cyclopropanecarboxamide mono-methanesulfonic acid salt; (R)—N-(4-(3-aminopiperidin-1-yl)-5-bromo-1H-pyrrolo[2,3-b]pyridin-3-yl)cyclopropanecarboxamide mono-ethanesulfonic acid salt; (R)—N-(4-(3-aminopiperidin-1-yl)-5-bromo-1H-pyrrolo[2,3-b]pyridin-3-yl)cyclopropanecarboxamide di-benzenesulfonic acid salt; (R)—N-(4-(3-aminopiperidin-1-yl)-5-bromo-1H-pyrrolo[2,3-b]pyridin-3-yl)cyclopropanecarboxamide di-toluenesulfonic acid salt; (R)—N-(4-(3-aminopiperidin-1-yl)-5-bromo-1H-pyrrolo[2,3-b]pyridin-3-yl)cyclopropanecarboxamide di-ethanesulfonic acid salt; (R)—N-(4-(3-aminopiperidin-1-yl)-5-bromo-1H-pyrrolo[2,3-b]pyridin-3-yl)cyclopropanecarboxamide maleic acid salt; (R)—N-(4-(3-aminopiperidin-1-yl)-5-bromo-1H-pyrrolo[2,3-b]pyridin-3-yl)cyclopropanecarboxamide HBr salt methanol solvate; (R)—N-(4-(3-aminopiperidin-1-yl)-5-bromo-1H-pyrrolo[2,3-b]pyridin-3-yl)cyclopropanecarboxamide di-HCl salt; and (R)—N-(4-(3-aminopiperidin-1-yl)-5-bromo-1H-pyrrolo[2,3-b]pyridin-3-yl)cyclopropanecarboxamide di-HBr salt; and b) instructions for use.

Accordingly, a further aspect of this invention provides a kit for treating a disorder, wherein said kit comprises a) a first pharmaceutical composition comprising a crystalline form of (R)—N-(4-(3-aminopiperidin-1-yl)-5-bromo-1H-pyrrolo[2,3-b]pyridin-3-yl)cyclopropanecarboxamide; and b) instructions for use. In some embodiments, the crystalline form is Form A. In some embodiments, the crystalline form is Form B.

In certain embodiments, the kit further comprises (c) a second pharmaceutical composition, wherein the second pharmaceutical composition comprises a second compound suitable for treating the disease. In certain embodiments comprising a second pharmaceutical composition, the kit further comprises instructions for the simultaneous, sequential or separate administration of said first and second pharmaceutical compositions to a patient in need thereof. In certain embodiments, said first and second pharmaceutical compositions are contained in separate containers. In other embodiments, said first and second pharmaceutical compositions are contained in the same container.

Although the crystalline forms of an acetic acid, ethanedisulfonic acid or fumaric acid salt of (R)—N-(4-(3-aminopiperidin-1-yl)-5-bromo-1H-pyrrolo[2,3-b]pyridin-3-yl)cyclopropanecarboxamide or hydrates thereof, are primarily of value as therapeutic agents for use in mammals, they are also useful whenever it is required to control CHK1 protein kinases, tyrosine kinases, additional serine/threonine kinases, and/or dual specificity kinases. Thus, they are useful as pharmacological standards for use in the development of new biological tests and in the search for new pharmacological agents and pharmaceutical forms thereof.

Although the crystalline forms of a compound selected from: (R)—N-(4-(3-aminopiperidin-1-yl)-5-bromo-1H-pyrrolo[2,3-b]pyridin-3-yl)cyclopropanecarboxamide, and pharmaceutically acceptable salts, solvates, and hydrates thereof, are primarily of value as therapeutic agents for use in mammals, they are also useful whenever it is required to control CHK1 protein kinases, tyrosine kinases, additional serine/threonine kinases, and/or dual specificity kinases. Thus, they are useful as pharmacological standards for use in the development of new biological tests and in the search for new pharmacological agents and pharmaceutical forms thereof.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to compounds described herein that are salts, it embraces pharmaceutically acceptable solvates and/or hydrates of such salts. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999.

### Crystalline Forms

Polymorphism is the ability of a substance to exist as two or more crystalline phases that have different arrangements and/or conformations of the molecules in the crystal lattice. Polymorphs show the same properties in the liquid or gaseous state but they may behave differently in the solid state.

Besides single-component polymorphs, drugs can also exist as salts and other multicomponent crystalline phases. For example, solvates and hydrates may contain an active pharmaceutical ingredient (API) host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily.

Recently, polymorph screens of 245 compounds revealed that about 90% of them exhibited multiple solid forms. Overall, approximately half the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026.)

The present invention is directed, inter alia, to crystalline forms of (R)—N-(4-(3-aminopiperidin-1-yl)-5-bromo-1H-pyrrolo[2,3-b]pyridin-3-yl)cyclopropanecarboxamide and salts, hydrates, and solvates thereof. The crystalline forms of the present invention can be identified by unique solid state signatures with respect to, for example, differential scanning calorimetry (DSC), X-ray powder diffraction (XRPD), and other solid state methods. Further characterization with respect to water or solvent content of the crystalline forms of the present invention can be gauged by any of the following methods for example, thermogravimetric analysis (TGA), DSC and the like. For DSC, it is known that the temperatures observed will depend upon sample purity, the rate of temperature change, as well as sample preparation technique and the particular instrument employed. Thus, the values reported herein relating to DSC thermograms can vary by about ±6° C. For desolvation events, DSC thermograms may vary by more than ±6° C. depending on instrument configuration and/or sample quantity. The values reported herein relating to DSC thermograms can also vary by about ±20 joules per gram. For XRPD, the relative intensities of the peaks can vary, depending upon the sample preparation technique, the sample mounting procedure and the particular instrument employed. Moreover, instrument variation and other factors can often affect the 2θ values. Therefore, the peak assignments of diffraction patterns can vary by about ±0.2 °2θ. The relative intensities of the reported peaks can also vary. For TGA, the features reported herein can vary by about ±5° C. The TGA features reported herein can also vary by about ±2% weight change due to, for example, sample variation. Further characterization with respect to hygroscopicity of the crystalline form can be gauged by, for example, dynamic vapor sorption (DVS). The DVS features reported herein can vary by about ±5% relative humidity. The DVS features reported herein can also vary by about ±5% weight change.

### Crystalline Form A of (R)—N-(4-(3-aminopiperidin-1-yl)-5-bromo-1H-pyrrolo[2,3-b]pyridin-3-yl)cyclopropanecarboxamide

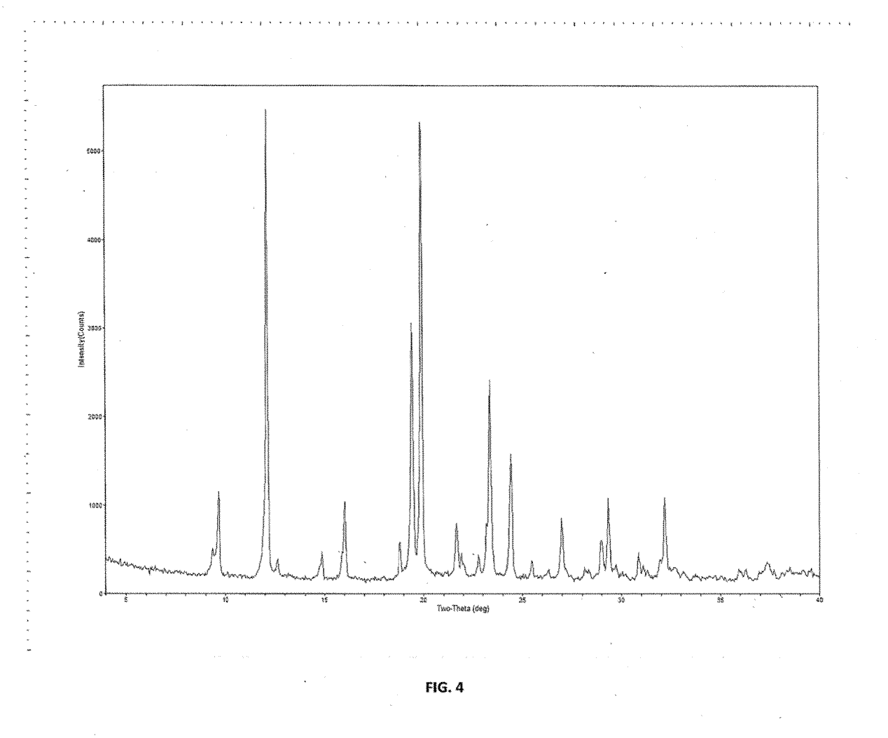

One aspect of the present invention is directed to a crystalline form of (R)—N-(4-(3-aminopiperidin-1-yl)-5-bromo-1H-pyrrolo[2,3-b]pyridin-3-yl)cyclopropanecarboxamide having an X-ray powder diffraction pattern comprising a peak, in terms of °2θ, at about 12.1. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 12.1, 19.9, and 19.5. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 12.1, 19.9, 19.5, 23.4, and 24.4. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 12.1, 19.9, 19.5, 23.4, 24.4, 9.7, and 29.4. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 9.7, 12.1, 16.1, 19.5, 19.9, 21.7, 23.4, 24.4, 27.0, 29.4, and 32.2. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising one or more peaks listed in Table 7. In some embodiments, the salt has an X-ray powder diffraction pattern substantially as shown in FIG. 4, wherein by “substantially” is meant that the reported peaks can vary by about ±0.2 °2θ and also that the relative intensities of the reported peaks can vary.

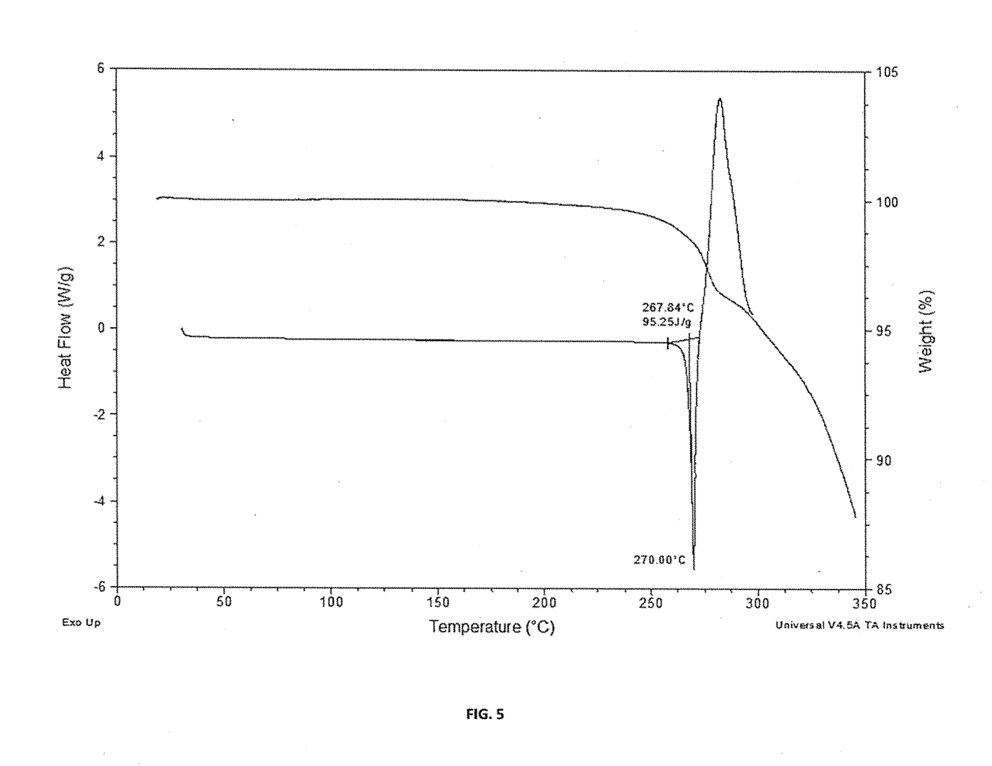

In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram comprising an endotherm with an extrapolated onset temperature between about 258° C. and about 278° C. In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram comprising an endotherm with an extrapolated onset temperature at about 268° C. In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram comprising an endotherm with an associated heat flow of about 95 joules per gram. In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram substantially as shown in FIG. 5, wherein by “substantially” is meant that the reported DSC features can vary by about ±6° C. and by about ±20 joules per gram.

In some embodiments, the crystalline form of (R)—N-(4-(3-aminopiperidin-1-yl)-5-bromo-1H-pyrrolo[2,3-b]pyridin-3-yl)cyclopropanecarboxamide has a thermogravimetric analysis profile substantially as shown in FIG. 5, wherein by “substantially” is meant that the reported TGA features can vary by about ±5° C. and by about ±2% weight change.

Form A of (R)—N-(4-(3-aminopiperidin-1-yl)-5-bromo-1H-pyrrolo[2,3-b]pyridin-3-yl)cyclopropanecarboxamide can be prepared by any of the suitable procedures known in the art for preparing crystalline polymorphs. In some embodiments Form A of (R)—N-(4-(3-aminopiperidin-1-yl)-5-bromo-1H-pyrrolo[2,3-b]pyridin-3-yl)cyclopropanecarboxamide salt can be prepared as described in Example 4. In some embodiments, Form A of (R)—N-(4-(3-aminopiperidin-1-yl)-5-bromo-1H-pyrrolo[2,3-b]pyridin-3-yl)cyclopropanecarboxamide can be prepared by heating (R)—N-(4-(3-aminopiperidin-1-yl)-5-bromo-1H-pyrrolo[2,3-b]pyridin-3-yl)cyclopropanecarboxamide containing one or more crystalline forms other than Form A. In some embodiments, Form A of (R)—N-(4-(3-aminopiperidin-1-yl)-5-bromo-1H-pyrrolo[2,3-b]pyridin-3-yl)cyclopropanecarboxamide can be prepared by recrystallizing crystalline (R)—N-(4-(3-aminopiperidin-1-yl)-5-bromo-1H-pyrrolo[2,3-b]pyridin-3-yl)cyclopropanecarboxamide containing one or more crystalline forms other than Form A.

### Crystalline Form B of (R)—N-(4-(3-aminopiperidin-1-yl)-5-bromo-1H-pyrrolo[2,3-b]pyridin-3-yl)cyclopropanecarboxamide

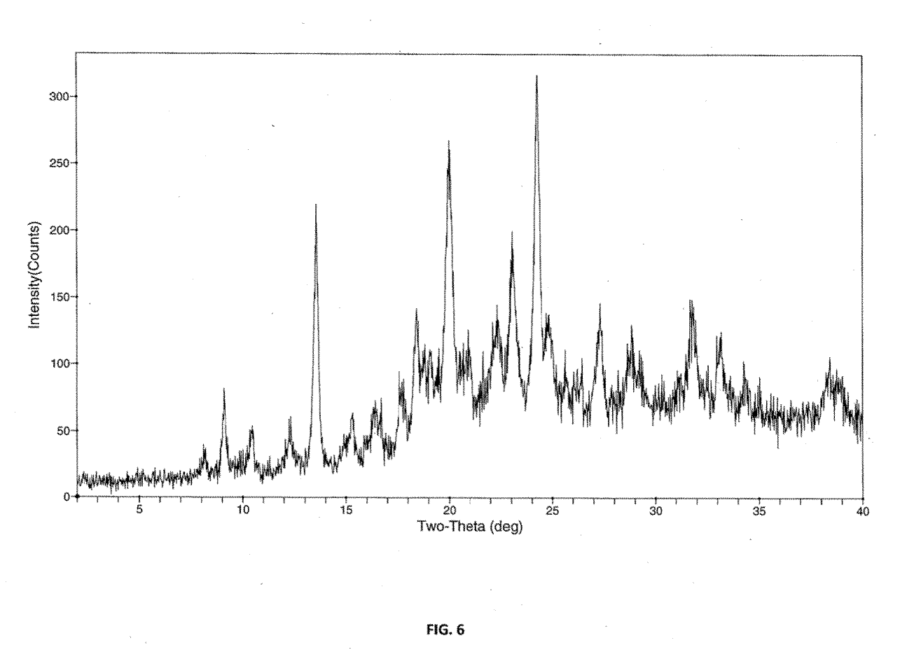

One aspect of the present invention is directed to a crystalline form of (R)—N-(4-(3-aminopiperidin-1-yl)-5-bromo-1H-pyrrolo[2,3-b]pyridin-3-yl)cyclopropanecarboxamide having an X-ray powder diffraction pattern comprising a peak, in terms of °2θ, at about 24.3. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 24.3, 20.0, and 13.6. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 24.3, 20.0, 13.6, 23.1, and 18.4. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 24.3, 20.0, 13.6, 23.1, 18.4, 31.8, and 27.3. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising peaks, in terms of °2θ, at about 9.1, 13.6, 18.4, 18.8, 20.0, 20.9, 23.1, 24.3, 27.3, 28.8, and 31.8. In some embodiments, the crystalline form of (R)—N-(4-(3-aminopiperidin-1-yl)-5-bromo-1H-pyrrolo[2,3-b]pyridin-3-yl)cyclopropanecarboxamide has an X-ray powder diffraction pattern comprising one or more peaks listed in Table 9. In some embodiments, the salt has an X-ray powder diffraction pattern substantially as shown in FIG. 6, wherein by “substantially” is meant that the reported peaks can vary by about ±0.2 °2θ and also that the relative intensities of the reported peaks can vary.

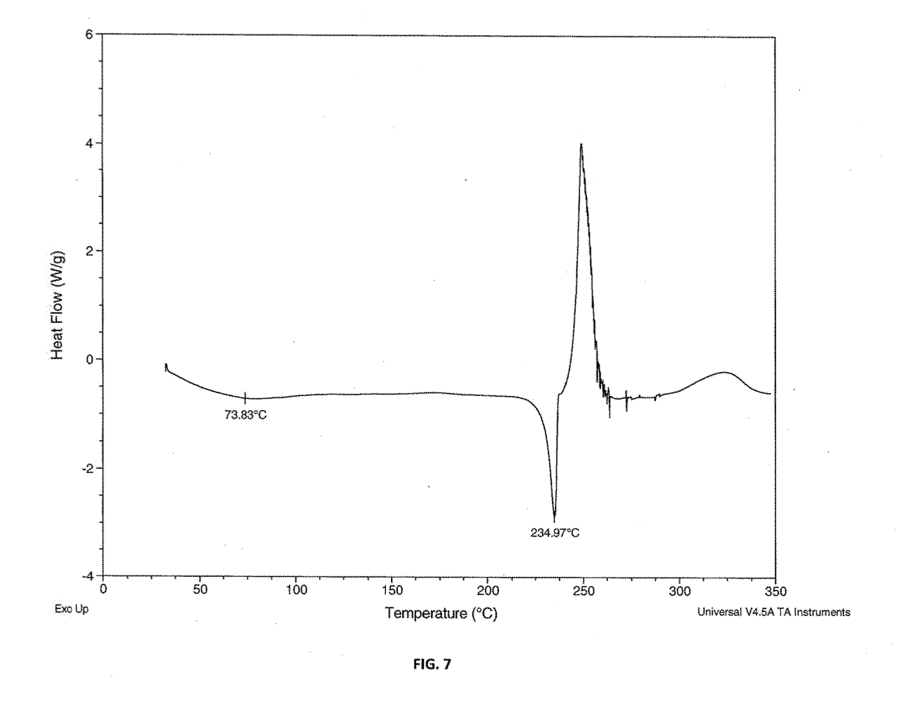

In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram comprising an endotherm with a peak between about 225° C. and about 245° C. In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram comprising an endotherm with a peak at about 235° C. In some embodiments, the crystalline form of (R)—N-(4-(3-aminopiperidin-1-yl)-5-bromo-1H-pyrrolo[2,3-b]pyridin-3-yl)cyclopropanecarboxamide has a differential scanning calorimetry thermogram substantially as shown in FIG. 7, wherein by “substantially” is meant that the reported DSC features can vary by about ±6° C. and by about ±20 joules per gram.

Form B of (R)—N-(4-(3-aminopiperidin-1-yl)-5-bromo-1H-pyrrolo[2,3-b]pyridin-3-yl)cyclopropanecarboxamide can be prepared by any of the suitable procedures known in the art for preparing crystalline polymorphs. In some embodiments Form B of (R)—N-(4-(3-aminopiperidin-1-yl)-5-bromo-1H-pyrrolo[2,3-b]pyridin-3-yl)cyclopropanecarboxamide salt can be prepared as described in Example 5. In some embodiments, Form B of (R)—N-(4-(3-aminopiperidin-1-yl)-5-bromo-1H-pyrrolo[2,3-b]pyridin-3-yl)cyclopropanecarboxamide can be prepared by heating (R)—N-(4-(3-aminopiperidin-1-yl)-5-bromo-1H-pyrrolo[2,3-b]pyridin-3-yl)cyclopropanecarboxamide containing one or more crystalline forms other than Form B. In some embodiments, Form B of (R)—N-(4-(3-aminopiperidin-1-yl)-5-bromo-1H-pyrrolo[2,3-b]pyridin-3-yl)cyclopropanecarboxamide can be prepared by recrystallizing crystalline (R)—N-(4-(3-aminopiperidin-1-yl)-5-bromo-1H-pyrrolo[2,3-b]pyridin-3-yl)cyclopropanecarboxamide containing one or more crystalline forms other than Form B.

### EXAMPLES

### General Experimental

XRPD: XRPD patterns were collected with a Rigaku SmartLab® diffractometer (Rigaku Corp., Tokyo, Japan), using an incident beam of Cu Kα (1.541904 Å) radiation generated using Cross Beam optics (40 kV×44 mA). Powder samples were packed using the top fill method onto zero-background holders and scans were acquired at a scan speed of 1° or 3.0°/min and step size of 0.02 or 0.04° 20 over 2-40 °2θ range in the bragg-brentano or parallel beam configuration (reflection geometry). Data was analyzed using commercial software (JADE®, version 9, Materials Data Inc., Livermore, Calif.).

XRPD data were also obtained using a Rigaku MiniFlexII (Rigaku Corp., Tokyo, Japan) diffractometer. The radiation used was CuKα (1.541837 Å) with voltage and current of 30 kV and 15 mA. Data was collected at ambient temperature from 2.0 to 40.0 °2θ using a step size of 0.020°. A low background sample holder was used and the stage was rotated at a revolution time of 1.0 seconds. The incident beam path was equipped with a 0.02 rad soller slit, 15 mm mask, 4° fixed anti-scatter slit and a programmable divergence slit. The diffracted beam was equipped with a 0.02 rad soller slit, programmable anti-scatter slit and a 0.02 mm nickel filter.

DSC general conditions: DSC analysis was conducted on a TA Instruments Q100 or Q2000 instrument. A sample size of approximately 1-5 mg was weighed out into a standard DSC pan; the pan was crimped. The sample was heated at 10° C./min from ambient temperature to 250-300° C. under dry nitrogen at 50 mL/min.

TGA general conditions: TGA was conducted on a TA Instruments Q500 instrument. A sample size of approximately 1-10 mg was used in a standard pan. The sample was heated at 10°/min from ambient temperature to 250-350° C. under dry nitrogen at 25 mL/min.

### Example 1

Acetic acid salt of (R)—N-(4-(3-aminopiperidin-1-yl)-5-bromo-1H-pyrrolo[2,3-b]pyridin-3-yl)cyclopropanecarboxamide: (R)—N-(4-(3-aminopiperidin-1-yl)-5-bromo-1H-pyrrolo[2,3-b]pyridin-3-yl)cyclopropanecarboxamide (104 mg) was suspended in ethyl acetate (15 mL). Acetic acid (1.32 mL of a 0.2 mol/L solution in ethyl acetate) was added. The suspension was stirred for 1 week and the solid isolated by centrifugation and then analyzed. The physical properties of the non-solvated acetic acid salt of (R)—N-(4-(3-aminopiperidin-1-yl)-5-bromo-1H-pyrrolo[2,3-b]pyridin-3-yl)cyclopropanecarboxamide are summarized in Table 1 below.

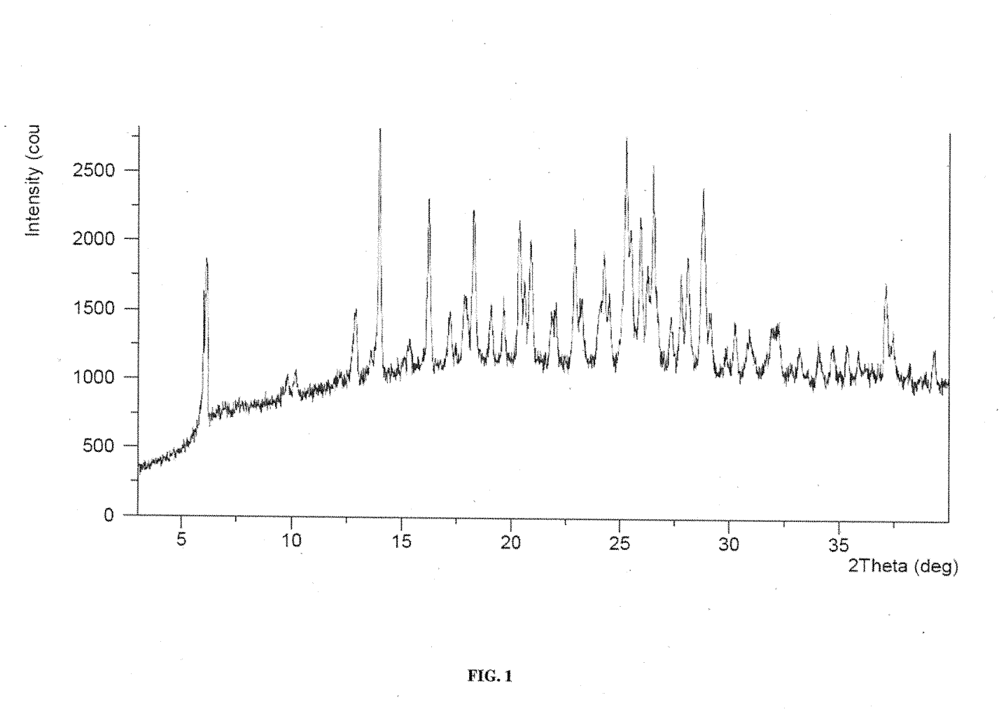

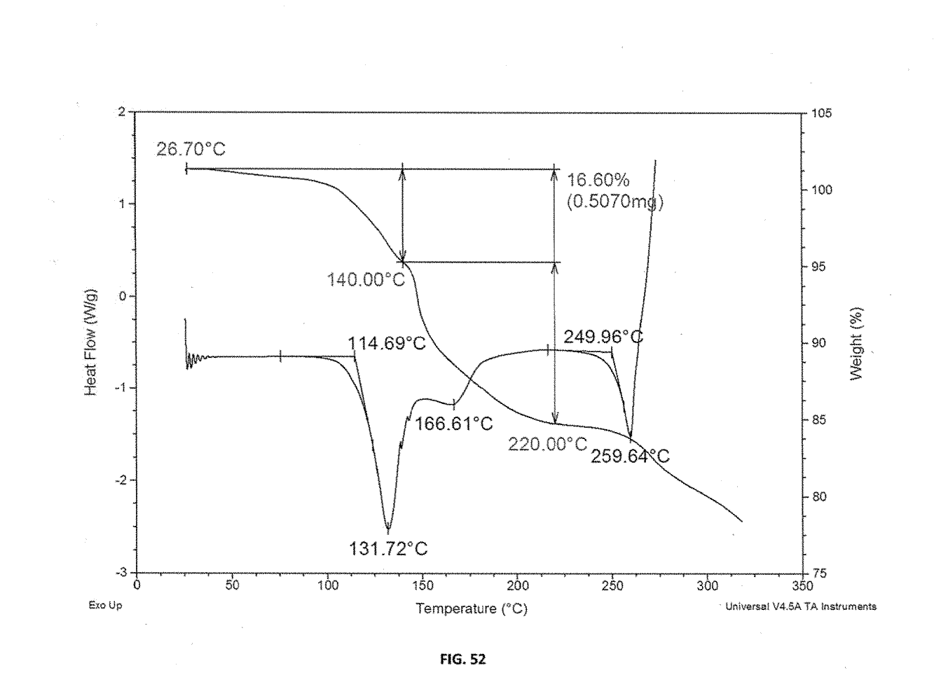

TABLE 1

| | |
|---|---|
| XRPD | FIG. 1: Peaks of ≥40% relative height at about 6.1, |
| | 14.0, 63.4, 18.3, 20.4, 20.9, 22.9, 24.3, 25.3, 25.5, |
| | 25.9, 26.5, and 28.8 °2θ. |
| DSC | FIG. 52: Endotherm with extrapolated onset temperature |
| | about 115° C. Endotherm at about 167° C. Endotherm with |
| | extrapolated onset temperature about 260° C. |
| TGA | FIG. 52: About 16.6% weight loss up to about 220° C. |

Certain XRPD diffraction peaks for the non-solvated acetic acid salt of (R)—N-(4-(3-aminopiperidin-1-yl)-5-bromo-1H-pyrrolo[2,3-b]pyridin-3-yl)cyclopropanecarboxamide are shown in Table 2 below.

TABLE 2
SCAN: 3.0055/39.9125/0.013/1 (sec), Cu, I (p) = 2941, 02/17/16 10:23 a
PEAK: 27 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 6.100 | 14.4774 | 694 | 1165 | 60.5 | 16310 | 46.5 | 0.155 |
| 2 | 9.816 | 9.0030 | 851 | 173 | 9.0 | 6372 | 18.2 | 0.407 |
| 3 | 10.191 | 8.6725 | 867 | 187 | 9.7 | 6521 | 18.6 | 0.385 |
| 4 | 12.923 | 6.8447 | 992 | 519 | 27.0 | 9460 | 27.0 | 0.201 |
| 5 | 13.576 | 6.5172 | 1005 | 204 | 10.6 | 5852 | 16.7 | 0.317 |
| 6 | 13.992 | 6.3242 | 1016 | 1925 | 100.0 | 25674 | 73.2 | 0.147 |
| 7 | 15.358 | 5.7649 | 1121 | 173 | 9.0 | 1882 | 5.4 | 0.120 |
| 8 | 16.239 | 5.4538 | 1097 | 1220 | 63.4 | 14429 | 41.1 | 0.131 |
| 9 | 17.203 | 5.1505 | 1169 | 321 | 16.7 | 3570 | 10.2 | 0.123 |
| 10 | 17.903 | 4.9505 | 1127 | 485 | 25.2 | 14808 | 42.2 | 0.338 |
| 11 | 18.282 | 4.8488 | 1172 | 1063 | 55.2 | 17346 | 49.4 | 0.180 |
| 12 | 19.097 | 4.6436 | 1148 | 396 | 20.5 | 5437 | 15.5 | 0.152 |
| 13 | 19.659 | 4.5122 | 1148 | 422 | 21.9 | 4810 | 13.7 | 0.126 |
| 14 | 20.387 | 4.3526 | 1150 | 998 | 51.8 | 17658 | 50.3 | 0.195 |
| 15 | 20.633 | 4.3013 | 1137 | 570 | 29.6 | 14078 | 40.1 | 0.273 |
| 16 | 20.894 | 4.2480 | 1134 | 880 | 45.7 | 17205 | 49.0 | 0.216 |
| 17 | 22.049 | 4.0281 | 1131 | 418 | 21.7 | 9363 | 26.7 | 0.248 |
| 18 | 22.908 | 3.8791 | 1132 | 973 | 50.5 | 20919 | 59.6 | 0.238 |
| 19 | 23.234 | 3.8253 | 1130 | 453 | 23.5 | 12979 | 37.0 | 0.317 |
| 20 | 24.066 | 3.6948 | 1134 | 404 | 21.0 | 16578 | 47.2 | 0.453 |
| 21 | 24.274 | 3.6638 | 1136 | 797 | 41.4 | 23288 | 66.4 | 0.323 |
| 22 | 24.508 | 3.6293 | 1138 | 475 | 24.6 | 14708 | 41.9 | 0.343 |
| 23 | 25.275 | 3.5209 | 1223 | 1577 | 81.9 | 35093 | 100.0 | 0.246 |
| 24 | 25.482 | 3.4927 | 1260 | 842 | 43.7 | 33509 | 95.5 | 0.440 |
| 25 | 25.913 | 3.4356 | 1180 | 1010 | 52.5 | 21026 | 59.9 | 0.230 |
| 26 | 26.250 | 3.3923 | 1180 | 632 | 32.8 | 14662 | 41.8 | 0.256 |
| 27 | 26.508 | 3.3598 | 1258 | 1294 | 67.2 | 21508 | 61.3 | 0.184 |
| 28 | 27.340 | 3.2595 | 1129 | 344 | 17.9 | 4394 | 12.5 | 0.141 |
| 29 | 27.771 | 3.2098 | 1138 | 639 | 33.2 | 9239 | 26.3 | 0.160 |
| 30 | 28.096 | 3.1735 | 1144 | 758 | 39.4 | 13673 | 39.0 | 0.199 |
| 31 | 28.784 | 3.0991 | 1120 | 1265 | 65.7 | 23997 | 68.4 | 0.210 |
| 32 | 29.148 | 3.0613 | 1096 | 401 | 20.9 | 7285 | 20.8 | 0.201 |
| 33 | 29.860 | 2.9898 | 1074 | 149 | 7.8 | 1872 | 5.3 | 0.139 |
| 34 | 30.228 | 2.9543 | 1068 | 370 | 19.2 | 6364 | 18.1 | 0.190 |
| 35 | 30.891 | 2.8923 | 1066 | 288 | 14.9 | 6602 | 18.8 | 0.254 |
| 36 | 31.619 | 2.8274 | 1064 | 111 | 5.8 | 3061 | 8.7 | 0.303 |
| 37 | 31.918 | 2.8016 | 1058 | 349 | 18.1 | 14746 | 42.0 | 0.467 |
| 38 | 32.255 | 2.7731 | 1053 | 376 | 19.5 | 14746 | 42.0 | 0.433 |
| 39 | 33.180 | 2.6978 | 1034 | 220 | 11.4 | 3939 | 11.2 | 0.198 |
| 40 | 34.050 | 2.6309 | 1011 | 274 | 14.2 | 3721 | 10.6 | 0.150 |
| 41 | 34.711 | 2.5823 | 1009 | 262 | 13.6 | 4729 | 13.5 | 0.199 |
| 42 | 35.338 | 2.5379 | 1036 | 241 | 12.5 | 3668 | 10.5 | 0.168 |
| 43 | 35.892 | 2.5000 | 1053 | 187 | 9.7 | 2440 | 7.0 | 0.144 |
| 44 | 36.481 | 2.4610 | 1040 | 109 | 5.7 | 2427 | 6.9 | 0.245 |
| 45 | 37.118 | 2.4202 | 1034 | 699 | 36.3 | 16168 | 46.1 | 0.255 |
| 46 | 37.442 | 2.3999 | 1008 | 369 | 19.2 | 12498 | 35.6 | 0.374 |
| 47 | 38.225 | 2.3526 | 990 | 158 | 8.2 | 1605 | 4.6 | 0.112 |
| 48 | 39.315 | 2.2898 | 985 | 248 | 12.9 | 5004 | 14.3 | 0.223 |

### Example 2 (R)—N-(4-(3-aminopiperidin-1-yl)-5-bromo-1H-pyrrolo[2,3-b]pyridin-3-yl)cyclopropanecarboxamide ethanedisulfonic acid hydrate

Method A: (R)—N-(4-(3-aminopiperidin-1-yl)-5-bromo-1H-pyrrolo[2,3-b]pyridin-3-yl)cyclopropanecarboxamide (101 mg) was suspended in ethyl acetate (15 mL). 1,2-Ethanedisulfonic acid (54.5 mg) was added and the suspension stirred for 1 day. The solid was isolated via centrifugation and then analyzed.

Method B: (R)—N-(4-(3-aminopiperidin-1-yl)-5-bromo-1H-pyrrolo[2,3-b]pyridin-3-yl)cyclopropanecarboxamide (˜15 g) was weighed into a 250 mL round-bottom flask, and then 230 mL of isopropanol was added. 1.0 equivalent of 1,2-ethanedisulfonic acid was slowly added into the sample. Then the suspension was kept stirring on a magnetic stirrer at room temperature for 24 hrs. After that, acetone was added in order to obtain more solid precipitation. The solid was isolated by vacuum filtering. The sample was purified with acetone and dried under reduced pressure at 40° C. overnight.

The physical properties of the hydrate of the ethanedisulfonic acid salt of (R)—N-(4-(3-aminopiperidin-1-yl)-5-bromo-1H-pyrrolo[2,3-b]pyridin-3-yl)cyclopropanecarboxamide are summarized in Table 3 below.

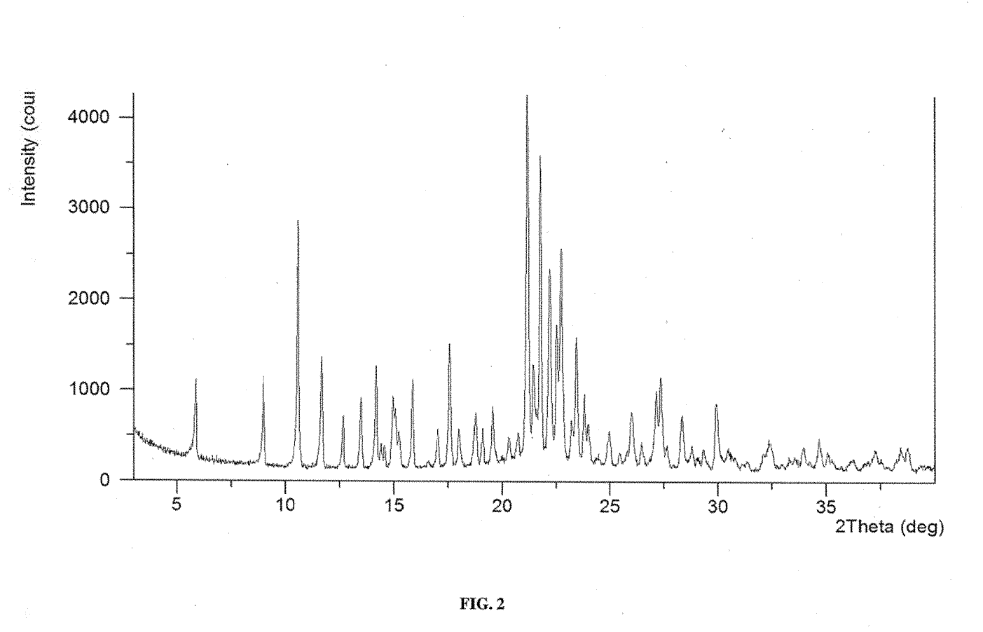

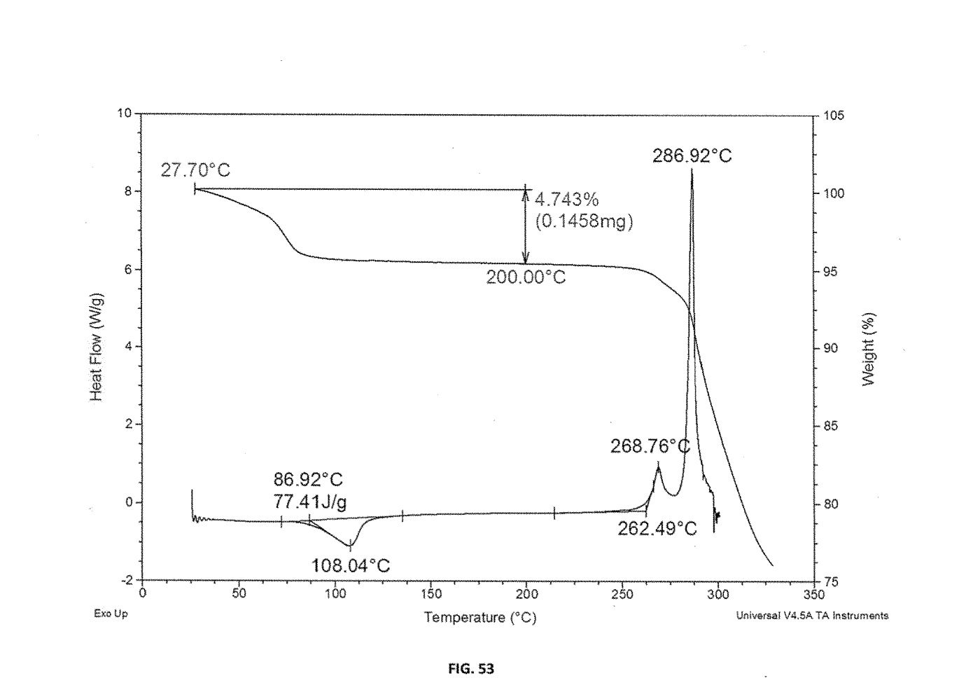

TABLE 3

| | |
|---|---|
| XRPD | FIG. 2: Peaks of ≥25% relative height at about 10.6, 11.7, 14.2, |
| | 17.6, 21.2, 21.8, 22.2, 22.5, 22.7, and 23.4 °2θ. |
| DSC | FIG. 53: Endotherm with extrapolated onset temperature about |
| | 87° C. and enthalpy of fusion about 77 J/g. Exotherm with |
| | extrapolated onset temperature about 262° C. |
| TGA | FIG. 53: About 4.7% weight loss up to about 200° C. |

Certain XRPD diffraction peaks for the non-solvated acetic acid salt of (R)—N-(4-(3-aminopiperidin-1-yl)-5-bromo-1H-pyrrolo[2,3-b]pyridin-3-yl)cyclopropanecarboxamide are shown in Table 4 below.

TABLE 4
SCAN: 3.0055/39.9125/0.013/1 (sec), Cu, I (p) = 4426, 02/17/16 10:25 a
PEAK: 21 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 5.852 | 15.0910 | 253 | 842 | 20.2 | 8732 | 18.4 | 0.115 |
| 2 | 8.984 | 9.8353 | 176 | 976 | 23.4 | 9148 | 19.2 | 0.104 |
| 3 | 10.595 | 8.3429 | 150 | 2638 | 63.3 | 25074 | 52.7 | 0.105 |
| 4 | 11.664 | 7.5805 | 148 | 1208 | 29.0 | 13250 | 27.9 | 0.121 |
| 5 | 12.650 | 6.9917 | 151 | 568 | 13.6 | 4978 | 10.5 | 0.097 |
| 6 | 13.471 | 6.5675 | 151 | 770 | 18.5 | 7647 | 16.1 | 0.110 |
| 7 | 14.185 | 6.2386 | 159 | 1114 | 26.7 | 11881 | 25.0 | 0.118 |
| 8 | 14.420 | 6.1374 | 164 | 256 | 6.1 | 3313 | 7.0 | 0.143 |
| 9 | 14.564 | 6.0771 | 153 | 247 | 5.9 | 3204 | 6.7 | 0.143 |
| 10 | 14.966 | 5.9150 | 167 | 777 | 18.6 | 15063 | 31.7 | 0.214 |
| 11 | 15.251 | 5.8048 | 168 | 373 | 9.0 | 5951 | 12.5 | 0.176 |
| 12 | 15.861 | 5.5829 | 159 | 952 | 22.9 | 8560 | 18.0 | 0.099 |
| 13 | 16.580 | 5.3424 | 164 | 66 | 1.6 | 491 | 1.0 | 0.083 |
| 14 | 17.021 | 5.2052 | 174 | 396 | 9.5 | 3968 | 8.3 | 0.111 |
| 15 | 17.591 | 5.0376 | 170 | 1354 | 32.5 | 15602 | 32.8 | 0.127 |
| 16 | 18.008 | 4.9219 | 174 | 404 | 9.7 | 5003 | 10.5 | 0.137 |
| 17 | 18.775 | 4.7225 | 205 | 557 | 13.4 | 6628 | 13.9 | 0.131 |
| 18 | 19.099 | 4.6432 | 204 | 379 | 9.1 | 3128 | 6.6 | 0.091 |
| 19 | 19.556 | 4.5358 | 224 | 587 | 14.1 | 6092 | 12.8 | 0.115 |
| 20 | 20.310 | 4.3689 | 259 | 225 | 5.4 | 2367 | 5.0 | 0.116 |
| 21 | 20.750 | 4.2772 | 223 | 320 | 7.7 | 4646 | 9.8 | 0.160 |
| 22 | 21.180 | 4.1915 | 259 | 4167 | 100.0 | 47577 | 100.0 | 0.126 |
| 23 | 21.466 | 4.1362 | 271 | 1028 | 24.7 | 22820 | 48.0 | 0.245 |
| 24 | 21.791 | 4.0753 | 271 | 3268 | 78.4 | 35338 | 74.3 | 0.119 |
| 25 | 22.207 | 3.9999 | 271 | 2079 | 49.9 | 28983 | 60.9 | 0.154 |
| 26 | 22.520 | 3.9450 | 271 | 1462 | 35.1 | 25680 | 54.0 | 0.194 |
| 27 | 22.740 | 3.9073 | 319 | 2256 | 54.1 | 33384 | 70.2 | 0.163 |
| 28 | 23.220 | 3.8275 | 271 | 394 | 9.4 | 7175 | 15.1 | 0.201 |
| 29 | 23.441 | 3.7919 | 260 | 1291 | 31.0 | 17720 | 37.2 | 0.152 |
| 30 | 23.818 | 3.7328 | 244 | 711 | 17.1 | 9284 | 19.5 | 0.144 |
| 31 | 24.025 | 3.7011 | 235 | 406 | 9.7 | 9318 | 19.6 | 0.253 |
| 32 | 24.482 | 3.6330 | 212 | 71 | 1.7 | 967 | 2.0 | 0.151 |
| 33 | 24.975 | 3.5624 | 197 | 361 | 8.7 | 4695 | 9.9 | 0.144 |
| 34 | 25.431 | 3.4996 | 195 | 126 | 3.0 | 1843 | 3.9 | 0.161 |
| 35 | 25.795 | 3.4510 | 194 | 156 | 3.8 | 5399 | 11.3 | 0.382 |
| 36 | 26.016 | 3.4223 | 194 | 561 | 13.5 | 11498 | 24.2 | 0.227 |
| 37 | 26.470 | 3.3645 | 204 | 233 | 5.6 | 2224 | 4.7 | 0.106 |
| 38 | 27.159 | 3.2807 | 184 | 792 | 19.0 | 21785 | 45.8 | 0.304 |
| 39 | 27.367 | 3.2562 | 179 | 973 | 23.4 | 19943 | 41.9 | 0.226 |
| 40 | 27.628 | 3.2260 | 172 | 234 | 5.6 | 3971 | 8.3 | 0.188 |
| 41 | 28.342 | 3.1464 | 181 | 556 | 13.4 | 8623 | 18.1 | 0.171 |
| 42 | 28.797 | 3.0977 | 162 | 232 | 5.6 | 5833 | 12.3 | 0.278 |
| 43 | 29.044 | 3.0719 | 163 | 117 | 2.8 | 1995 | 4.2 | 0.189 |
| 44 | 29.344 | 3.0413 | 195 | 166 | 4.0 | 1988 | 4.2 | 0.133 |
| 45 | 29.941 | 2.9819 | 163 | 696 | 16.7 | 10780 | 22.7 | 0.171 |
| 46 | 30.486 | 2.9299 | 161 | 215 | 5.2 | 7550 | 15.9 | 0.388 |
| 47 | 30.762 | 2.9042 | 155 | 122 | 2.9 | 6641 | 14.0 | 0.601 |
| 48 | 31.125 | 2.8711 | 158 | 50 | 1.2 | 1532 | 3.2 | 0.339 |
| 49 | 31.384 | 2.8480 | 149 | 77 | 1.8 | 1532 | 3.2 | 0.220 |
| 50 | 32.099 | 2.7862 | 146 | 177 | 4.2 | 8796 | 18.5 | 0.550 |
| 51 | 32.424 | 2.7590 | 149 | 289 | 6.9 | 9870 | 20.7 | 0.377 |
| 52 | 32.932 | 2.7176 | 159 | 47 | 1.1 | 802 | 1.7 | 0.187 |
| 53 | 33.286 | 2.6895 | 164 | 104 | 2.5 | 2741 | 5.8 | 0.290 |
| 54 | 33.543 | 2.6695 | 164 | 117 | 2.8 | 3361 | 7.1 | 0.318 |
| 55 | 33.971 | 2.6369 | 171 | 215 | 5.2 | 4667 | 9.8 | 0.240 |
| 56 | 34.255 | 2.6156 | 188 | 46 | 1.1 | 476 | 1.0 | 0.114 |
| 57 | 34.673 | 2.5850 | 175 | 304 | 7.3 | 4597 | 9.7 | 0.167 |
| 58 | 35.053 | 2.5579 | 163 | 180 | 4.3 | 4641 | 9.8 | 0.285 |
| 59 | 35.271 | 2.5425 | 157 | 94 | 2.3 | 3419 | 7.2 | 0.402 |
| 60 | 36.246 | 2.4764 | 144 | 116 | 2.8 | 2631 | 5.5 | 0.251 |
| 61 | 36.705 | 2.4464 | 147 | 65 | 1.6 | 2488 | 5.2 | 0.423 |
| 62 | 37.246 | 2.4121 | 146 | 212 | 5.1 | 7887 | 16.6 | 0.410 |
| 63 | 37.508 | 2.3959 | 146 | 112 | 2.7 | 4546 | 9.6 | 0.449 |
| 64 | 38.235 | 2.3520 | 145 | 105 | 2.5 | 4349 | 9.1 | 0.457 |
| 65 | 38.418 | 2.3412 | 144 | 247 | 5.9 | 7053 | 14.8 | 0.315 |
| 66 | 38.743 | 2.3223 | 142 | 242 | 5.8 | 6371 | 13.4 | 0.290 |

### Example 3

(R)—N-(4-(3-aminopiperidin-1-yl)-5-bromo-1H-pyrrolo[2,3-b]pyridin-3-yl)cyclopropanecarboxamide fumaric acid salt hydrate: (R)—N-(4-(3-aminopiperidin-1-yl)-5-bromo-1H-pyrrolo[2,3-b]pyridin-3-yl)cyclopropanecarboxamide (102 mg) was suspended in ethyl acetate (15 mL). Fumaric acid (34.5 mg) was added and the suspension stirred for 1 day. The solid was isolated by centrifugation and dried at room temperature under vacuum. The solid was exposed to 100% RH at room temperature for 3 days and then analyzed.

The physical properties of the hydrate of the fumaric acid salt of (R)—N-(4-(3-aminopiperidin-1-yl)-5-bromo-1H-pyrrolo[2,3-b]pyridin-3-yl)cyclopropanecarboxamide are summarized in Table 5 below.

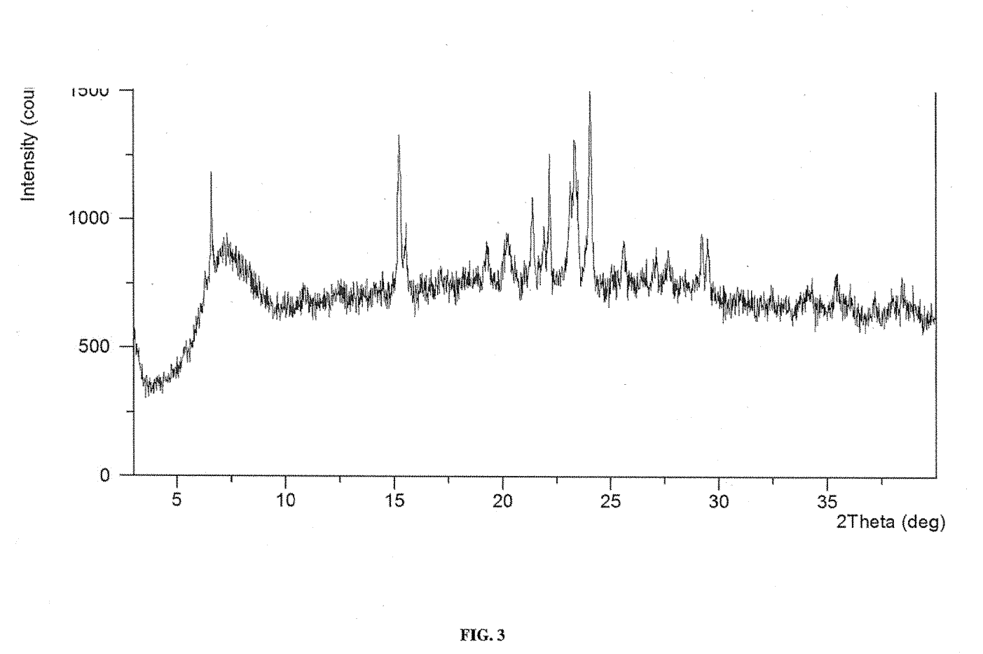

TABLE 5

| | |
|---|---|
| XRPD | FIG. 3. |

### Example 4

(R)—N-(4-(3-aminopiperidin-1-yl)-5-bromo-1H-pyrrolo[2,3-b]pyridin-3-yl)cyclopropanecarboxamide Form A: (R)—N-(4-(3-aminopiperidin-1-yl)-5-bromo-1H-pyrrolo[2,3-b]pyridin-3-yl)cyclopropanecarboxamide (28.6 mg) was dissolved in propyl acetate (6 mL) in a 20 mL vial. The material was allowed to evaporate at ambient to give Form A. Form A is a non-solvated crystalline form of the free base of (R)—N-(4-(3-aminopiperidin-1-yl)-5-bromo-1H-pyrrolo[2,3-b]pyridin-3-yl)cyclopropanecarboxamide. The physical properties of Form A are summarized in Table 6 below.

TABLE 6

| | |
|---|---|
| Aqueous | 0.1N HCl: 49.9; FedSIF pH 5: 10.2; FasSIF pH 6.8: 2.4; |
| Solubility | H₂O: 0.12 |
| (mg/mL) | |
| XRPD | FIG. 4: Peaks of ≥10% relative height at about 9.7, 12.1, |
| | 16.1, 19.5, 19.9, 21.7, 23.4, 24.4, 27.0, 29.4, and 32.2 °2θ. |
| DSC | FIG. 5: Endotherm with extrapolated onset temperature about |
| | 268° C. and enthalpy of fusion about 95 J/g. |
| TGA | FIG. 5: No weight loss until melt/degradation. |

Certain XRPD diffraction peaks for (R)—N-(4-(3-aminopiperidin-1-yl)-5-bromo-1H-pyrrolo[2,3-b]pyridin-3-yl)cyclopropanecarboxamide Form A are shown in Table 7 below.

TABLE 7
SCAN: 4.0/40.0/0.04/1 (sec), Cu (40 kV, 44 mA), I (p) = 5467,
01/12/16 02:48 p
PEAK: 11 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 9.371 | 9.4294 | 216 | 297 | 5.6 | 2546 | 12.2 | 0.291 |
| 2 | 9.677 | 9.1324 | 214 | 935 | 17.8 | 5164 | 24.8 | 0.188 |
| 3 | 12.146 | 7.2807 | 199 | 5268 | 100.0 | 18551 | 89.0 | 0.120 |
| 4 | 12.660 | 6.9864 | 215 | 185 | 3.5 | 1159 | 5.6 | 0.213 |
| 5 | 14.873 | 5.9517 | 174 | 302 | 5.7 | 1295 | 6.2 | 0.146 |
| 6 | 16.061 | 5.5138 | 168 | 876 | 16.6 | 4268 | 20.5 | 0.166 |
| 7 | 18.828 | 4.7094 | 171 | 416 | 7.9 | 1536 | 7.4 | 0.125 |
| 8 | 19.452 | 4.5596 | 211 | 2846 | 54.0 | 13626 | 65.4 | 0.163 |
| 9 | 19.932 | 4.4510 | 223 | 5108 | 97.0 | 20846 | 100.0 | 0.139 |
| 10 | 21.681 | 4.0957 | 217 | 578 | 11.0 | 2871 | 13.8 | 0.169 |
| 11 | 21.927 | 4.0502 | 215 | 250 | 4.8 | 1499 | 7.2 | 0.204 |
| 12 | 22.807 | 3.8959 | 217 | 225 | 4.3 | 907 | 4.4 | 0.137 |
| 13 | 23.396 | 3.7992 | 216 | 2195 | 41.7 | 10647 | 51.1 | 0.165 |
| 14 | 24.435 | 3.6400 | 209 | 1372 | 26.0 | 5520 | 26.5 | 0.137 |
| 15 | 25.480 | 3.4929 | 194 | 186 | 3.5 | 606 | 2.9 | 0.111 |
| 16 | 26.341 | 3.3807 | 191 | 91 | 1.7 | 291 | 1.4 | 0.109 |
| 17 | 27.011 | 3.2983 | 182 | 673 | 12.8 | 3405 | 16.3 | 0.172 |
| 18 | 28.173 | 3.1649 | 187 | 112 | 2.1 | 514 | 2.5 | 0.157 |
| 19 | 29.008 | 3.0757 | 206 | 407 | 7.7 | 2359 | 11.3 | 0.197 |
| 20 | 29.364 | 3.0392 | 188 | 890 | 16.9 | 5866 | 28.1 | 0.224 |
| 21 | 29.752 | 3.0004 | 190 | 144 | 2.7 | 1708 | 8.2 | 0.402 |
| 22 | 30.891 | 2.8923 | 182 | 288 | 5.5 | 1054 | 5.1 | 0.124 |
| 23 | 32.211 | 2.7768 | 206 | 880 | 16.7 | 5107 | 24.5 | 0.197 |
| 24 | 32.724 | 2.7344 | 204 | 99 | 1.9 | 1591 | 7.6 | 0.547 |
| 25 | 35.973 | 2.4945 | 163 | 118 | 2.2 | 755 | 3.6 | 0.218 |
| 26 | 36.313 | 2.4719 | 165 | 119 | 2.3 | 865 | 4.1 | 0.246 |
| 27 | 36.981 | 2.4288 | 171 | 83 | 1.6 | 1024 | 4.9 | 0.421 |
| 28 | 37.363 | 2.4048 | 178 | 181 | 3.4 | 2093 | 10.0 | 0.393 |
| 29 | 37.719 | 2.3830 | 183 | 90 | 1.7 | 532 | 2.5 | 0.202 |
| 30 | 38.517 | 2.3354 | 206 | 101 | 1.9 | 993 | 4.8 | 0.334 |
| 31 | 39.196 | 2.2965 | 219 | 52 | 1.0 | 881 | 4.2 | 0.575 |

### Example 5

(R)—N-(4-(3-aminopiperidin-1-yl)-5-bromo-1H-pyrrolo[2,3-b]pyridin-3-yl)cyclopropanecarboxamide Form B: (R)—N-(4-(3-aminopiperidin-1-yl)-5-bromo-1H-pyrrolo[2,3-b]pyridin-3-yl)cyclopropanecarboxamide (24.6 mg) was dissolved in ethyl formate (5 mL) in a 20 mL scintillation vial at room temperature. The solution was filtered and placed into a clean 20 mL scintillation vial. The vial top was left open and the solution evaporated under ambient conditions. The sample could be heated to 130° C. in a TGA under nitrogen (10° C./min to 140° C. and then cooled to ambient) to remove solvent. Form B is a non-solvated crystalline form of the free base of (R)—N-(4-(3-aminopiperidin-1-yl)-5-bromo-1H-pyrrolo[2,3-b]pyridin-3-yl)cyclopropanecarboxamide. The physical properties of Form B are summarized in Table 8 below.

TABLE 8

| | |
|---|---|
| XRPD | FIG. 6: Peaks of ≥25% relative height at about 9.1, 13.6, |
| | 18.4, 18.8, 20.0, 20.9, 23.1, 24.3, 27.3, 28.8, and 31.8 °2θ. |
| DSC | FIG. 7: Endotherm at about 235° C. |

Certain XRPD diffraction peaks for (R)—N-(4-(3-aminopiperidin-1-yl)-5-bromo-1H-pyrrolo[2,3-b]pyridin-3-yl)cyclopropanecarboxamide, Form B are shown in Table 9 below.

TABLE 9
SCAN: 2.0/40.0/0.02/0.6 (sec), Cu (30 kV, 15 mA), I (p) = 317.0,
11/02/12 01:49 p
PEAK: 25 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 9.102 | 9.7081 | 21 | 61 | 25.4 | 599 | 10.1 | 0.166 |
| 2 | 10.460 | 8.4505 | 23 | 31 | 12.9 | 294 | 5.0 | 0.161 |
| 3 | 12.341 | 7.1662 | 22 | 39 | 16.4 | 609 | 10.3 | 0.263 |
| 4 | 13.561 | 6.5242 | 25 | 195 | 80.9 | 2610 | 44.1 | 0.228 |
| 5 | 15.338 | 5.7721 | 27 | 37 | 15.4 | 778 | 13.1 | 0.356 |
| 6 | 16.303 | 5.4326 | 33 | 35 | 14.3 | 829 | 14.0 | 0.408 |
| 7 | 18.439 | 4.8079 | 53 | 89 | 37.0 | 1999 | 33.8 | 0.381 |
| 8 | 18.838 | 4.7070 | 53 | 62 | 25.8 | 1141 | 19.3 | 0.312 |
| 9 | 19.081 | 4.6475 | 56 | 55 | 22.6 | 2027 | 34.2 | 0.631 |
| 10 | 20.000 | 4.4359 | 56 | 212 | 87.7 | 5919 | 100.0 | 0.476 |
| 11 | 20.678 | 4.2920 | 56 | 60 | 24.7 | 1860 | 31.4 | 0.531 |
| 12 | 20.923 | 4.2423 | 56 | 70 | 28.8 | 1226 | 20.7 | 0.300 |
| 13 | 22.339 | 3.9765 | 87 | 58 | 24.1 | 922 | 15.6 | 0.270 |
| 14 | 23.062 | 3.8534 | 85 | 115 | 47.6 | 1770 | 29.9 | 0.262 |
| 15 | 24.279 | 3.6629 | 76 | 241 | 100.0 | 5108 | 86.3 | 0.360 |
| 16 | 24.858 | 3.5790 | 83 | 55 | 23.0 | 1381 | 23.3 | 0.424 |
| 17 | 27.320 | 3.2617 | 72 | 74 | 30.7 | 875 | 14.8 | 0.201 |
| 18 | 28.839 | 3.0933 | 69 | 61 | 25.3 | 1466 | 24.8 | 0.409 |
| 19 | 31.783 | 2.8132 | 71 | 78 | 32.3 | 1977 | 33.4 | 0.431 |
| 20 | 33.143 | 2.7008 | 70 | 51 | 21.1 | 1016 | 17.2 | 0.340 |
| 21 | 38.416 | 2.3413 | 63 | 43 | 17.9 | 1171 | 19.8 | 0.460 |
| 22 | 38.857 | 2.3157 | 62 | 29 | 12.1 | 777 | 13.1 | 0.453 |

### Example 6 (R)—N-(4-(3-aminopiperidin-1-yl)-5-bromo-1H-pyrrolo[2,3-b]pyridin-3-yl)cyclopropanecarboxamide cyclopropyl methyl ether solvate

Method A: (R)—N-(4-(3-aminopiperidin-1-yl)-5-bromo-1H-pyrrolo[2,3-b]pyridin-3-yl)cyclopropanecarboxamide (27.2 mg) was dissolved in cyclopentyl methyl ether (7.5 mL) in a 20 mL scintillation vial at room temperature. The solution was filtered and placed into a clean 20 mL scintillation vial. The vial top was left open and the solution evaporated under ambient conditions.

Method B: (R)—N-(4-(3-aminopiperidin-1-yl)-5-bromo-1H-pyrrolo[2,3-b]pyridin-3-yl)cyclopropanecarboxamide was dissolved at 200 mg/mL in pyridine 65° C. The solution was diluted to 10 mg/mL with cyclopentyl methyl ether and cooled from 65° C. to 10° C. over 14.5 h. The mixture was allowed to evaporate until dry to yield the cyclopentyl methyl ether solvate.

The physical properties of the cyclopropyl methyl ether solvate of the free base of (R)—N-(4-(3-aminopiperidin-1-yl)-5-bromo-1H-pyrrolo[2,3-b]pyridin-3-yl)cyclopropanecarboxamide are summarized in Table 10 below.

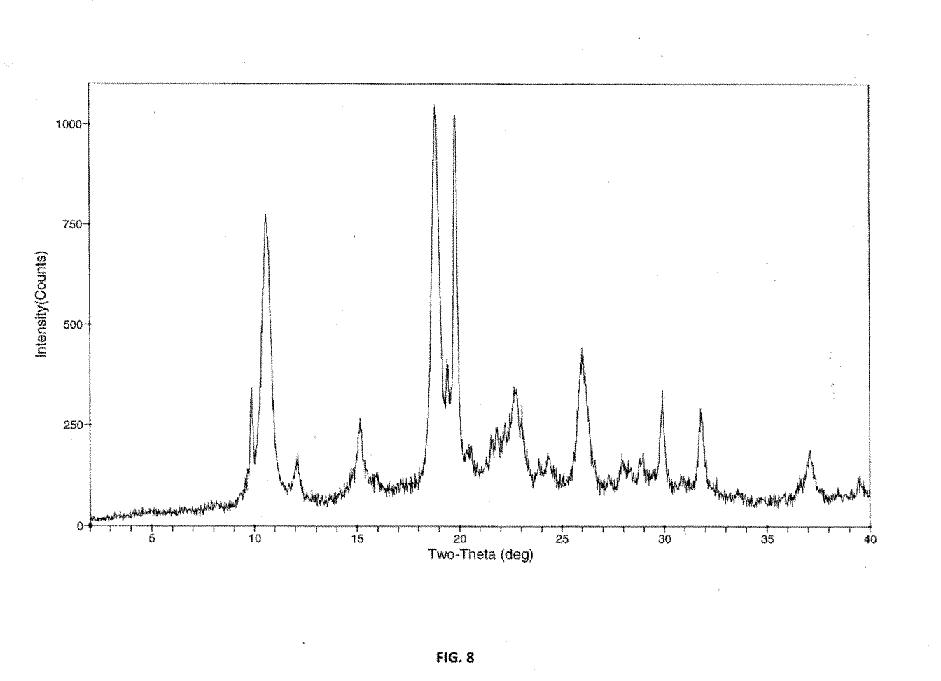

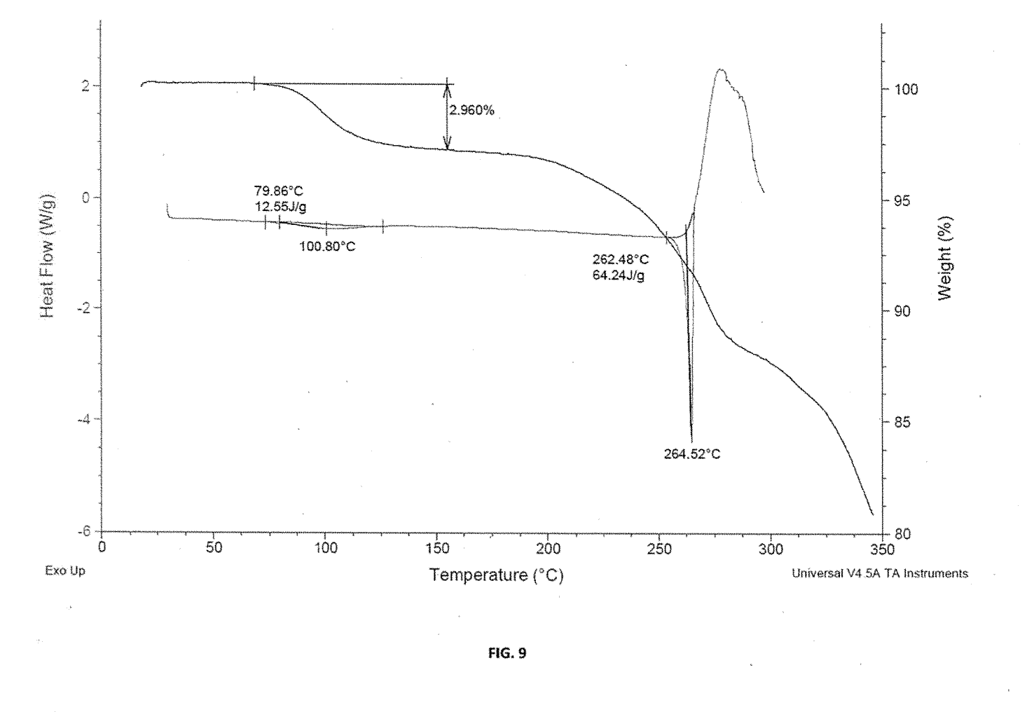

TABLE 10

| | |
|---|---|
| XRPD | FIG. 8: Peaks of ≥10% relative height at about 9.9, 10.6, 12.1, |
| | 15.2, 18.9, 19.8, 21.8, 22.7, 23.1, 26.0, 29.9, 31.8, and |
| | 37.1 °2θ. |
| DSC | FIG. 9: Endotherm with extrapolated onset temperature about |
| | 80° C. and enthalpy of fusion about 13 J/g. Endotherm with |
| | extrapolated onset temperature about 262° C. and enthalpy of |
| | fusion about 64 J/g. |
| TGA | FIG. 9: About 3.0% weight loss up to about 160° C. |

Certain XRPD diffraction peaks for the cyclopropyl methyl ether solvate of the free base of (R)—N-(4-(3-aminopiperidin-1-yl)-5-bromo-1H-pyrrolo[2,3-b]pyridin-3-yl)cyclopropanecarboxamide are shown in Table 11 below.

TABLE 11
SCAN: 2.0/40.0/0.02/0.6 (sec), Cu (30 kV, 15 mA), I (p) = 1048,
10/24/12 11:28 a
PEAK: 27 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 9.877 | 8.9479 | 61 | 280 | 30.0 | 3364 | 14.7 | 0.204 |
| 2 | 10.600 | 8.3391 | 105 | 671 | 71.9 | 18612 | 81.5 | 0.471 |
| 3 | 12.119 | 7.2971 | 85 | 94 | 10.1 | 927 | 4.1 | 0.168 |
| 4 | 15.160 | 5.8396 | 100 | 167 | 17.9 | 3160 | 13.8 | 0.321 |
| 5 | 18.860 | 4.7014 | 114 | 934 | 100.0 | 22831 | 100.0 | 0.416 |
| 6 | 19.802 | 4.4800 | 141 | 882 | 94.5 | 13810 | 60.5 | 0.266 |
| 7 | 21.803 | 4.0730 | 132 | 114 | 12.2 | 2233 | 9.8 | 0.333 |
| 8 | 22.236 | 3.9946 | 163 | 93 | 9.9 | 4418 | 19.4 | 0.809 |
| 9 | 22.681 | 3.9172 | 116 | 230 | 24.6 | 10284 | 45.0 | 0.760 |
| 10 | 23.057 | 3.8542 | 116 | 143 | 15.3 | 5002 | 21.9 | 0.594 |
| 11 | 23.863 | 3.7259 | 114 | 55 | 5.8 | 1557 | 6.8 | 0.486 |
| 12 | 24.302 | 3.6595 | 112 | 69 | 7.4 | 1557 | 6.8 | 0.385 |
| 13 | 26.001 | 3.4242 | 109 | 335 | 35.8 | 9055 | 39.7 | 0.460 |
| 14 | 27.944 | 3.1903 | 111 | 72 | 7.7 | 975 | 4.3 | 0.231 |
| 15 | 28.377 | 3.1426 | 110 | 37 | 4.0 | 1889 | 8.3 | 0.862 |
| 16 | 28.943 | 3.0824 | 120 | 52 | 5.5 | 619 | 2.7 | 0.204 |
| 17 | 29.919 | 2.9840 | 109 | 228 | 24.5 | 2837 | 12.4 | 0.211 |
| 18 | 31.783 | 2.8132 | 96 | 195 | 20.9 | 3113 | 13.6 | 0.272 |
| 19 | 36.603 | 2.4530 | 75 | 54 | 5.8 | 780 | 3.4 | 0.246 |
| 20 | 37.119 | 2.4201 | 69 | 121 | 13.0 | 3293 | 14.4 | 0.463 |

### Example 7 (R)—N-(4-(3-aminopiperidin-1-yl)-5-bromo-1H-pyrrolo[2,3-b]pyridin-3-yl)cyclopropanecarboxamide 1,2-dichloroethane solvate

Method A: (R)—N-(4-(3-aminopiperidin-1-yl)-5-bromo-1H-pyrrolo[2,3-b]pyridin-3-yl)cyclopropanecarboxamide (29.8 mg) was dissolved in 1,2-dichloroethane (4 mL) in a 20 mL scintillation vial at room temperature. The solution was filtered and placed into a clean 20 mL scintillation vial. The vial top was left open and the solution evaporated under ambient conditions.

Method B: (R)—N-(4-(3-aminopiperidin-1-yl)-5-bromo-1H-pyrrolo[2,3-b]pyridin-3-yl)cyclopropanecarboxamide was dissolve at 33.3 mg/mL in 2:1 (v/v) 1,2-dichloroethane:methanol at 65° C. The solution was cooled from 65° C. to 10° C. over 14.5 h, then evaporated to until dry to yield (R)—N-(4-(3-aminopiperidin-1-yl)-5-bromo-1H-pyrrolo[2,3-b]pyridin-3-yl)cyclopropanecarboxamide 1,2-dichloroethane solvate.

The physical properties of the 1,2-dichloroethane solvate of the free base of (R)—N-(4-(3-aminopiperidin-1-yl)-5-bromo-1H-pyrrolo[2,3-b]pyridin-3-yl)cyclopropanecarboxamide are summarized in Table 12 below.

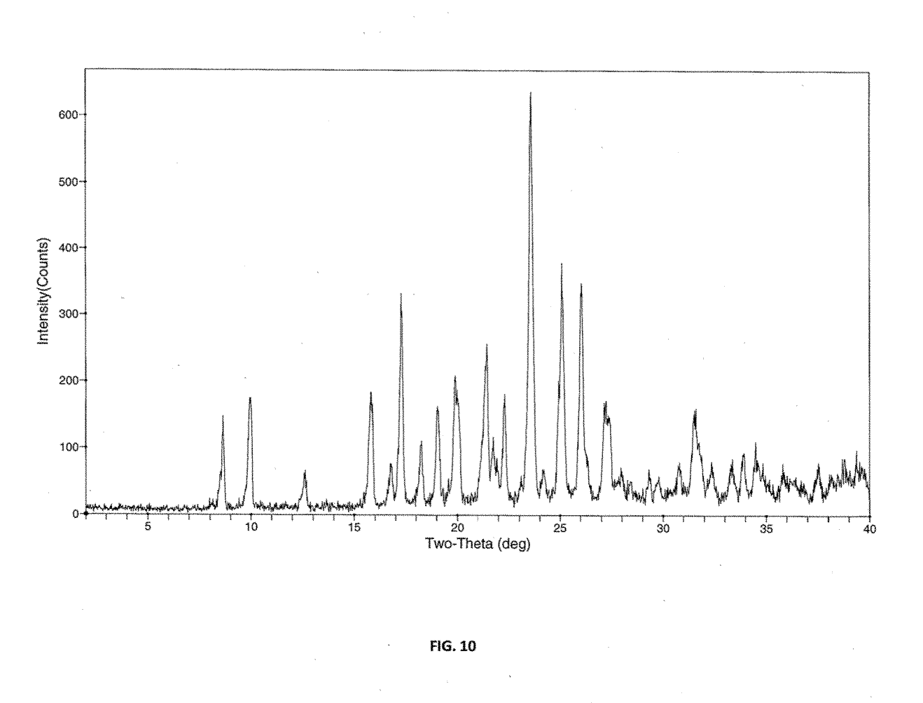

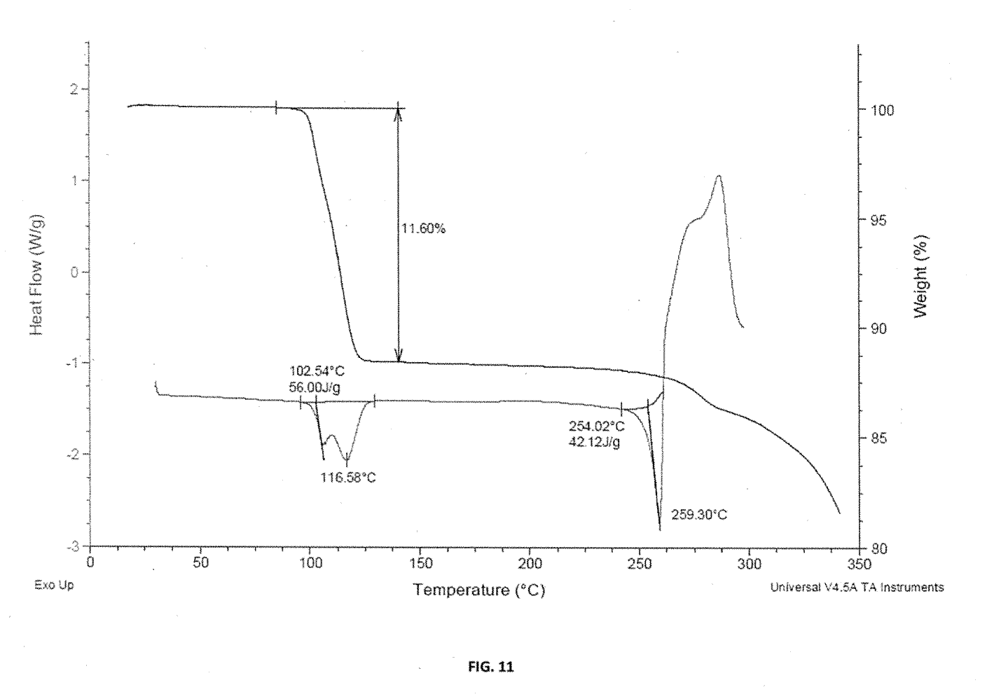

TABLE 12

| | |
|---|---|
| XRPD | FIG. 10: Peaks of ≥12% relative height at about 8.6, 9.9, 15.8, |
| | 17.3, 19.9, 21.3, 21.8, 23.6, 25.1, 26.0, 27.1, 31.5, and |
| | 31.9 °2θ. |
| DSC | FIG. 11: Endotherm with extrapolated onset temperature about |
| | 103° C. and enthalpy of fusion about 56 J/g. Endotherm with |
| | extrapolated onset temperature about 254° C. and enthalpy of |
| | fusion about 42 J/g. |
| TGA | FIG. 11: About 11.6% weight loss up to about 145° C. |

Certain XRPD diffraction peaks for the 1,2-dichloroethane solvate of the free base of (R)—N-(4-(3-aminopiperidin-1-yl)-5-bromo-1H-pyrrolo[2,3-b]pyridin-3-yl)cyclopropanecarboxamide are shown in Table 13 below.

TABLE 13
SCAN: 2.0/40.0/0.02/0.6 (sec), Cu (30 kV, 15 mA), I (p) = 1112,
10/24/12 11:47 a
PEAK: 17 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 8.635 | 10.2324 | 9 | 131 | 12.2 | 1439 | 12.8 | 0.186 |
| 2 | 9.882 | 8.9437 | 10 | 169 | 15.7 | 1620 | 14.4 | 0.163 |
| 3 | 12.612 | 7.0130 | 12 | 82 | 7.6 | 842 | 7.5 | 0.174 |
| 4 | 15.780 | 5.6115 | 17 | 160 | 14.8 | 1676 | 14.9 | 0.178 |
| 5 | 16.726 | 5.2960 | 21 | 85 | 7.9 | 871 | 7.8 | 0.174 |
| 6 | 17.258 | 5.1341 | 23 | 400 | 37.1 | 4107 | 36.6 | 0.175 |
| 7 | 18.262 | 4.8541 | 23 | 122 | 11.3 | 1038 | 9.3 | 0.145 |
| 8 | 19.042 | 4.6570 | 22 | 125 | 11.6 | 1327 | 11.8 | 0.180 |
| 9 | 19.484 | 4.5522 | 25 | 31 | 2.9 | 563 | 5.0 | 0.306 |
| 10 | 19.900 | 4.4579 | 35 | 676 | 62.7 | 4949 | 44.1 | 0.124 |
| 11 | 21.323 | 4.1636 | 30 | 210 | 19.5 | 2872 | 25.6 | 0.232 |
| 12 | 21.781 | 4.0772 | 31 | 135 | 12.6 | 1619 | 14.4 | 0.203 |
| 13 | 22.242 | 3.9935 | 43 | 108 | 10.1 | 954 | 8.5 | 0.150 |
| 14 | 23.581 | 3.7697 | 34 | 1078 | 100.0 | 11212 | 100.0 | 0.177 |
| 15 | 24.222 | 3.6715 | 40 | 49 | 4.6 | 387 | 3.5 | 0.134 |
| 16 | 25.081 | 3.5477 | 40 | 413 | 38.3 | 5700 | 50.8 | 0.235 |
| 17 | 25.999 | 3.4244 | 40 | 401 | 37.2 | 5083 | 45.3 | 0.215 |
| 18 | 27.140 | 3.2829 | 36 | 152 | 14.1 | 2916 | 26.0 | 0.326 |
| 19 | 27.378 | 3.2550 | 39 | 101 | 9.3 | 2062 | 18.4 | 0.348 |
| 20 | 27.780 | 3.2088 | 38 | 32 | 3.0 | 411 | 3.7 | 0.218 |
| 21 | 27.985 | 3.1858 | 34 | 62 | 5.8 | 927 | 8.3 | 0.254 |
| 22 | 29.258 | 3.0499 | 36 | 30 | 2.8 | 196 | 1.7 | 0.111 |
| 23 | 30.820 | 2.8989 | 52 | 142 | 13.2 | 1078 | 9.6 | 0.129 |
| 24 | 31.537 | 2.8346 | 42 | 270 | 25.1 | 5076 | 45.3 | 0.319 |
| 25 | 31.859 | 2.8066 | 35 | 135 | 12.6 | 3485 | 31.1 | 0.438 |
| 26 | 32.321 | 2.7676 | 35 | 85 | 7.8 | 984 | 8.8 | 0.198 |
| 27 | 33.336 | 2.6856 | 38 | 55 | 5.1 | 556 | 5.0 | 0.173 |
| 28 | 33.902 | 2.6420 | 42 | 79 | 7.4 | 750 | 6.7 | 0.161 |
| 29 | 34.555 | 2.5936 | 36 | 77 | 7.1 | 1633 | 14.6 | 0.363 |
| 30 | 34.937 | 2.5661 | 38 | 33 | 3.1 | 635 | 5.7 | 0.325 |
| 31 | 35.761 | 2.5088 | 35 | 50 | 4.6 | 1005 | 9.0 | 0.345 |
| 32 | 36.393 | 2.4667 | 34 | 34 | 3.2 | 930 | 8.3 | 0.465 |
| 33 | 37.981 | 2.3671 | 34 | 44 | 4.1 | 774 | 6.9 | 0.301 |
| 34 | 38.437 | 2.3401 | 40 | 50 | 4.7 | 1703 | 15.2 | 0.576 |
| 35 | 38.779 | 2.3203 | 40 | 45 | 4.2 | 899 | 8.0 | 0.337 |
| 36 | 39.277 | 2.2920 | 40 | 58 | 5.4 | 1190 | 10.6 | 0.347 |
| 37 | 39.539 | 2.2774 | 40 | 49 | 4.6 | 1190 | 10.6 | 0.410 |

### Example 8

(R)—N-(4-(3-aminopiperidin-1-yl)-5-bromo-1H-pyrrolo[2,3-b]pyridin-3-yl)cyclopropanecarboxamide 2-methyltetrahydrofuran solvate: (R)—N-(4-(3-aminopiperidin-1-yl)-5-bromo-1H-pyrrolo[2,3-b]pyridin-3-yl)cyclopropanecarboxamide (28.1 mg) was dissolved in 2-methyltetrahydrofuran (2 mL) in a 20 mL scintillation vial at room temperature. The solution was filtered and placed into a clean 20 mL scintillation vial. The vial top was left open and the solution evaporated under ambient conditions.

The physical properties of the 2-methyltetrahydrofuran solvate of the free base of (R)—N-(4-(3-aminopiperidin-1-yl)-5-bromo-1H-pyrrolo[2,3-b]pyridin-3-yl)cyclopropanecarboxamide are summarized in Table 14 below.

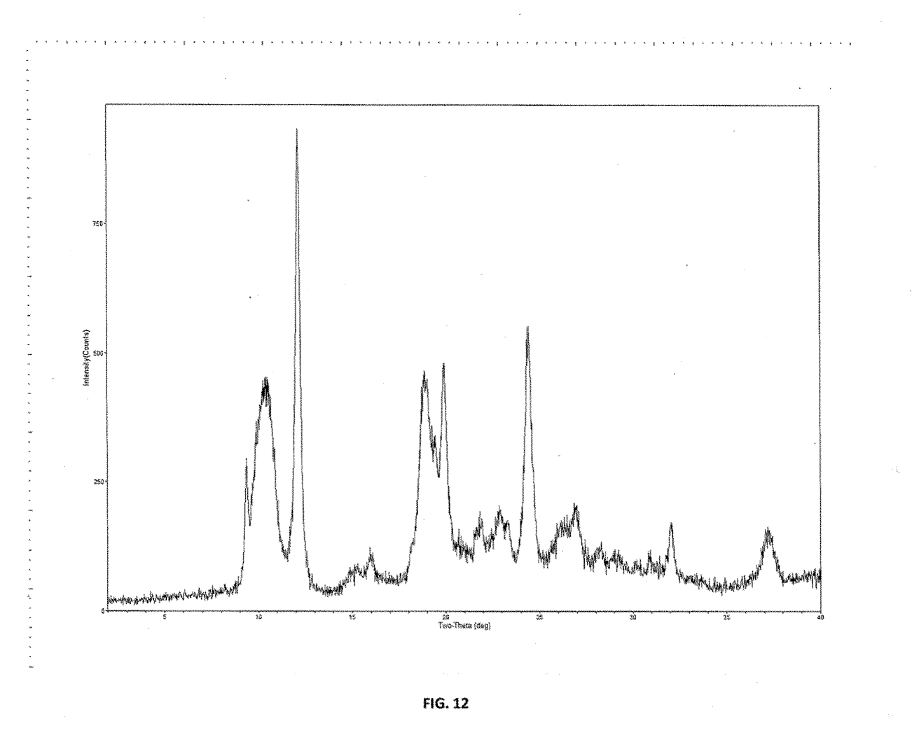

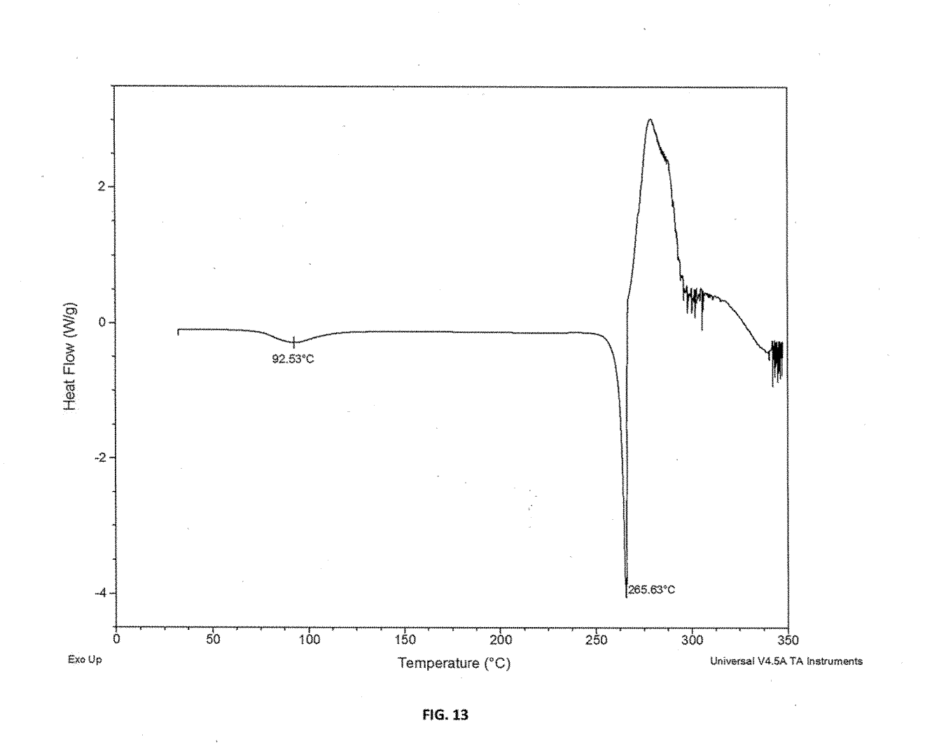

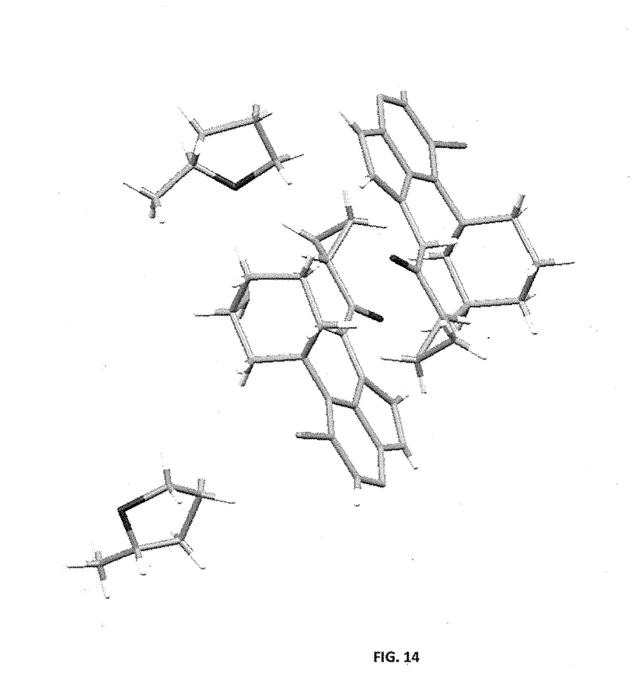

TABLE 14

| | |
|---|---|
| XRPD | FIG. 12: Peaks of ≥5% relative height at about 9.4, 10.5, 12.2, |
| | 18.9, 19.9, 22.9, 24.5, 26.2, 26.9, 32.0, and 37.2 °2θ. |
| DSC | FIG. 13: Endotherms at about 93° C. and about 266° C. |
| SCXRD | FIG. 14. |

Certain XRPD diffraction peaks for the 2-methyltetrahydrofuran solvate of the free base of (R)—N-(4-(3-aminopiperidin-1-yl)-5-bromo-1H-pyrrolo[2,3-b]pyridin-3-yl)cyclopropanecarboxamide are shown in Table 15 below.

TABLE 15
SCAN: 2.0/40.0/0.02/0.6 (sec), Cu (30 kV, 15 mA), I (p) = 934.0,
10/25/12 09:24 a
PEAK: 45 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 9.400 | 9.4009 | 37 | 258 | 29.0 | 3707 | 20.8 | 0.244 |
| 2 | 10.521 | 8.4016 | 109 | 345 | 38.8 | 17692 | 99.1 | 0.871 |
| 3 | 12.161 | 7.2723 | 45 | 889 | 100.0 | 16226 | 90.9 | 0.310 |
| 4 | 18.920 | 4.6866 | 71 | 396 | 44.6 | 17856 | 100.0 | 0.766 |
| 5 | 19.921 | 4.4535 | 71 | 410 | 46.1 | 9713 | 54.4 | 0.402 |
| 6 | 22.903 | 3.8798 | 119 | 86 | 9.7 | 2627 | 14.7 | 0.517 |
| 7 | 24.460 | 3.6363 | 103 | 449 | 50.5 | 10024 | 56.1 | 0.380 |
| 8 | 26.195 | 3.3993 | 125 | 51 | 5.7 | 2223 | 12.4 | 0.741 |
| 9 | 26.901 | 3.3116 | 118 | 92 | 10.3 | 3336 | 18.7 | 0.619 |
| 10 | 32.043 | 2.7909 | 72 | 101 | 11.4 | 1808 | 10.1 | 0.304 |
| 11 | 37.221 | 2.4137 | 64 | 100 | 11.3 | 3275 | 18.3 | 0.555 |

For single crystal X-ray diffraction, (R)—N-(4-(3-aminopiperidin-1-yl)-5-bromo-1H-pyrrolo[2,3-b]pyridin-3-yl)cyclopropanecarboxamide was dissolved at 500 mg/mL in 2,2,2-trifluoroethanol at 65° C. The solution was diluted to 25 mg/mL with 2-methyltetrahydrofuran and cooled from 65° C. to 10° C. over 14.5 h. Crystals for diffraction were pulled directly from resulting saturated solution. A colorless plate of C₁₆H₂₀BrN₅O having approximate dimensions of 0.20×0.20×0.12 mm was mounted on a fiber in a random orientation. Preliminary examination and data collection were performed Cu Kₐ radiation (1=1.54184 Å) on a Rigaku Rapid II equipped with confocal optics. The data were collected at a temperature of 150(1)K. Cell constants for data collection were obtained from least-squares refinement, using the setting angles of 35,637 reflections in the range 3<q<70°. The space group was determined by the program XPREP. There were no systematic absences; the space group was determined to be P−1(#2). A total of 35,637 reflections were collected, of which 7,284 were unique. Frames were integrated using program CrystalClear. Lorentz and polarization corrections were applied to the data. An empirical absorption correction using CrystalClear was applied. Transmission coefficients ranged from 0.425 to 0.716. Intensities of equivalent reflections were averaged. The structure was solved by direct methods using Charge Flipping in PLATON. The remaining atoms were located in succeeding difference Fourier syntheses. Hydrogen atoms were included in the refinement but restrained to ride on the atom to which they are bonded. The structure was refined in full-matrix least-squares where the function minimized was Sw(|Fo|²−|Fc|²)² and the weight w is defined as 1/[s²(Fo²)+(0.1012P)²+10.7676P] where P=(Fo²+2Fc²)/3. Scattering factors were taken from the “International Tables for Crystallography”. Residual electron density was adjusted using the SQUEEZE option in PLATON. Refinement was performed on a LINUX PC using SHELX-97. The crystal structure of the asymmetric unit of the 2-methyltetrahydrofuran solvate is shown in FIG. 14.

### Example 9

(R)—N-(4-(3-aminopiperidin-1-yl)-5-bromo-1H-pyrrolo[2,3-b]pyridin-3-yl)cyclopropanecarboxamide 1-pentanol solvate: (R)—N-(4-(3-aminopiperidin-1-yl)-5-bromo-1H-pyrrolo[2,3-b]pyridin-3-yl)cyclopropanecarboxamide (26.5 mg) was dissolved in 1-pentanol (1.5 mL) in a 20 mL scintillation vial at room temperature. The solution was filtered and placed into a clean 20 mL scintillation vial. The vial top was left open and the solution evaporated under ambient conditions. The physical properties of the 1-pentanol solvate of the free base of (R)—N-(4-(3-aminopiperidin-1-yl)-5-bromo-1H-pyrrolo[2,3-b]pyridin-3-yl)cyclopropanecarboxamide are summarized in Table 16 below.

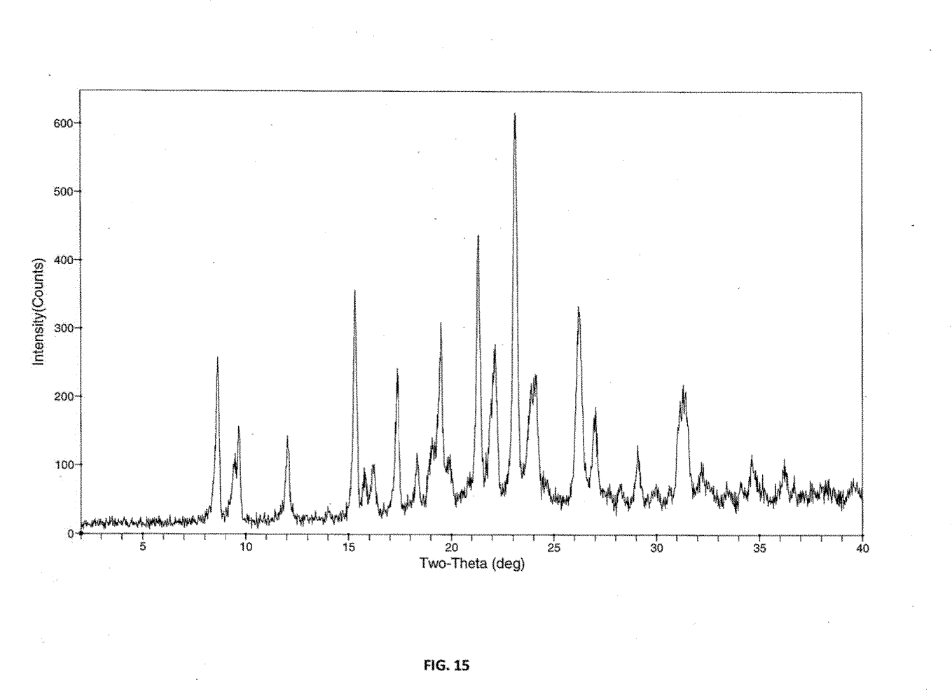

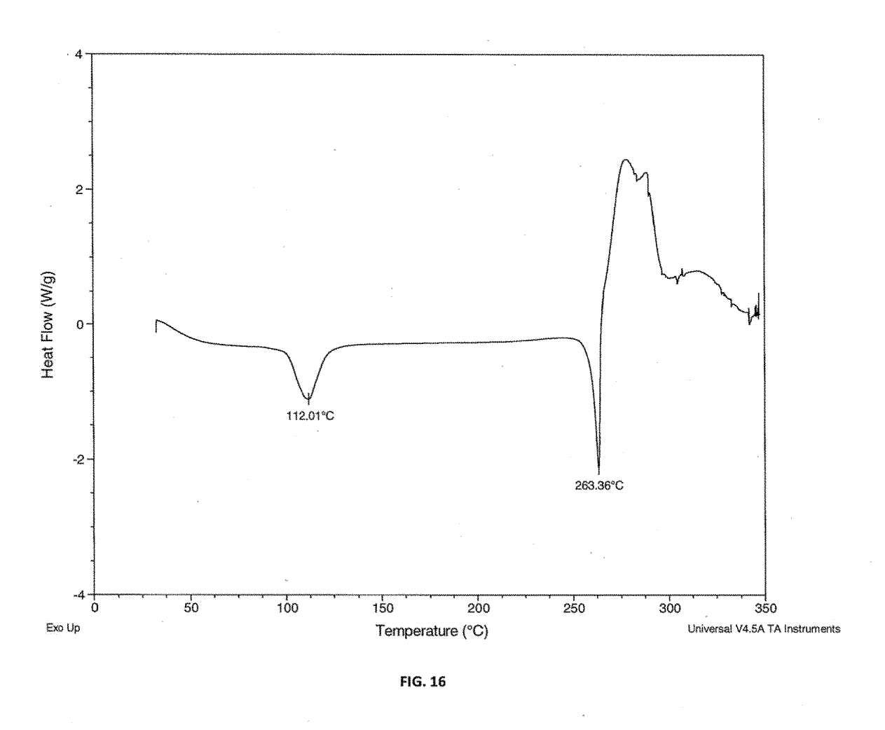

TABLE 16

| | |
|---|---|
| XRPD | FIG. 15: Peaks of ≥25% relative height at about 8.6, 9.7, 15.3, |
| | 17.4, 19.5, 21.4, 22.2, 23.1, 24.1, 26.2, 27.0, and 31.4 °2θ. |
| DSC | FIG. 16: Endotherms at about 112° C. and about 263° C. |

Certain XRPD diffraction peaks for the 1-pentanol solvate of the free base of (R)—N-(4-(3-aminopiperidin-1-yl)-5-bromo-1H-pyrrolo[2,3-b]pyridin-3-yl)cyclopropanecarboxamide are shown in Table 17 below.

TABLE 17
SCAN: 2.0/40.0/0.02/0.6 (sec), Cu (30 kV, 15 mA), I (p) = 617.0,
10/25/12 09:43 a
PEAK: 21 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 8.640 | 10.2264 | 23 | 235 | 44.0 | 2785 | 42.8 | 0.202 |
| 2 | 9.462 | 9.3394 | 23 | 84 | 15.8 | 2453 | 37.7 | 0.494 |
| 3 | 9.661 | 9.1471 | 16 | 141 | 26.4 | 2572 | 39.6 | 0.310 |
| 4 | 12.041 | 7.3440 | 21 | 122 | 22.8 | 1531 | 23.5 | 0.214 |
| 5 | 15.340 | 5.7716 | 24 | 333 | 62.5 | 3734 | 57.4 | 0.191 |
| 6 | 15.764 | 5.6172 | 33 | 64 | 12.0 | 1008 | 15.5 | 0.267 |
| 7 | 16.237 | 5.4544 | 33 | 69 | 13.0 | 1001 | 15.4 | 0.246 |
| 8 | 17.381 | 5.0982 | 37 | 205 | 38.6 | 2300 | 35.4 | 0.190 |
| 9 | 18.342 | 4.8330 | 50 | 68 | 12.8 | 464 | 7.1 | 0.115 |
| 10 | 19.021 | 4.6620 | 47 | 82 | 15.4 | 3022 | 46.5 | 0.624 |
| 11 | 19.501 | 4.5484 | 51 | 259 | 48.6 | 5129 | 78.9 | 0.336 |
| 12 | 19.936 | 4.4500 | 51 | 66 | 12.4 | 1469 | 22.6 | 0.378 |
| 13 | 20.899 | 4.2471 | 51 | 40 | 7.5 | 455 | 7.0 | 0.193 |
| 14 | 21.356 | 4.1572 | 71 | 367 | 68.9 | 4393 | 67.6 | 0.203 |
| 15 | 22.160 | 4.0082 | 76 | 202 | 37.9 | 2975 | 45.8 | 0.250 |
| 16 | 23.121 | 3.8437 | 84 | 533 | 100.0 | 6502 | 100.0 | 0.207 |
| 17 | 24.101 | 3.6896 | 52 | 182 | 34.1 | 5195 | 79.9 | 0.485 |
| 18 | 26.221 | 3.3959 | 49 | 285 | 53.5 | 5358 | 82.4 | 0.319 |
| 19 | 27.036 | 3.2953 | 48 | 138 | 26.0 | 2228 | 34.3 | 0.274 |
| 20 | 29.081 | 3.0681 | 46 | 84 | 15.7 | 971 | 14.9 | 0.197 |
| 21 | 29.975 | 2.9786 | 47 | 27 | 5.1 | 303 | 4.7 | 0.188 |
| 22 | 31.437 | 2.8434 | 64 | 145 | 27.2 | 3730 | 57.4 | 0.438 |
| 23 | 32.198 | 2.7779 | 61 | 45 | 8.4 | 697 | 10.7 | 0.266 |
| 24 | 33.426 | 2.6786 | 46 | 32 | 6.1 | 345 | 5.3 | 0.182 |
| 25 | 34.101 | 2.6271 | 48 | 30 | 5.7 | 414 | 6.4 | 0.233 |
| 26 | 34.639 | 2.5875 | 47 | 70 | 13.0 | 1621 | 24.9 | 0.396 |
| 27 | 36.222 | 2.4780 | 52 | 59 | 11.1 | 901 | 13.9 | 0.258 |
| 28 | 39.557 | 2.2764 | 55 | 28 | 5.2 | 335 | 5.2 | 0.206 |

### Example 10

(R)—N-(4-(3-aminopiperidin-1-yl)-5-bromo-1H-pyrrolo[2,3-b]pyridin-3-yl)cyclopropanecarboxamide pyridine solvate: (R)—N-(4-(3-aminopiperidin-1-yl)-5-bromo-1H-pyrrolo[2,3-b]pyridin-3-yl)cyclopropanecarboxamide (29.9 mg) was dissolved in pyridine (1 mL) in a 20 mL scintillation vial at room temperature. The solution was filtered and placed into a clean 20 mL scintillation vial. The vial top was left open and the solution evaporated under ambient conditions. The physical properties of the pyridine solvate of the free base of (R)—N-(4-(3-aminopiperidin-1-yl)-5-bromo-1H-pyrrolo[2,3-b]pyridin-3-yl)cyclopropanecarboxamide are summarized in Table 18 below.

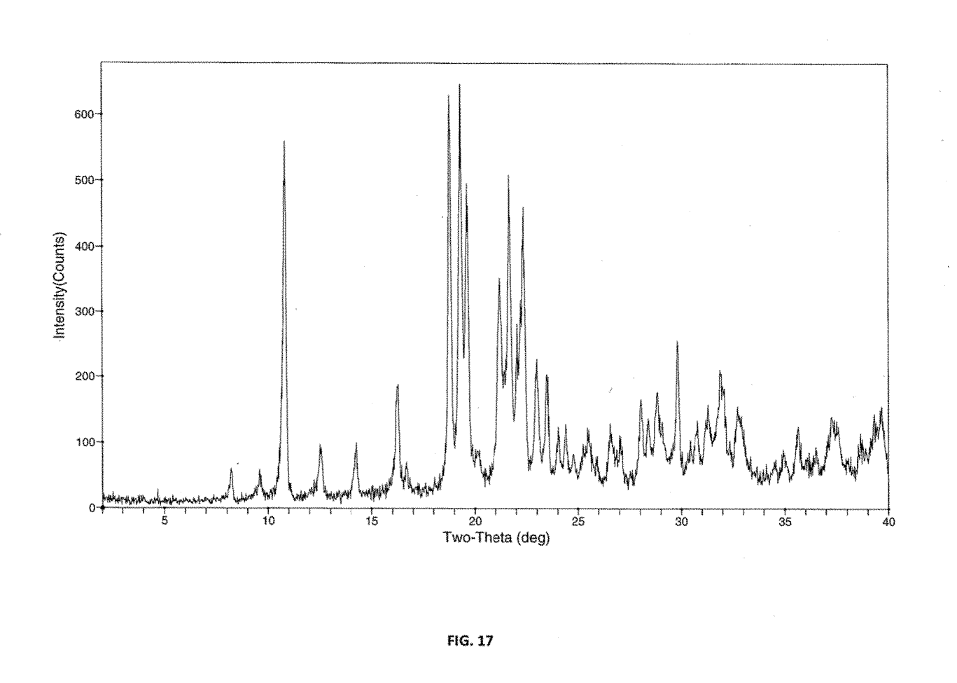

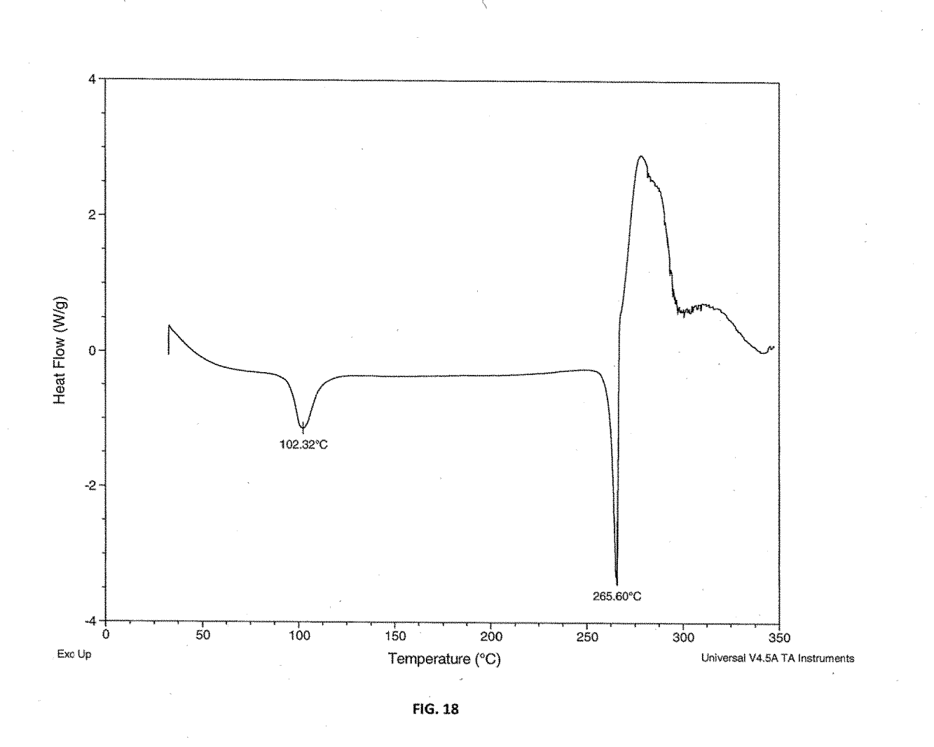

TABLE 18

| | |
|---|---|
| XRPD | FIG. 17: Peaks of ≥25% relative height at about 10.8, 16.3, |
| | 18.8, 19.3, 19.6, 21.2, 21.7, 22.4, 23.0, 29.8, and 31.9 °2θ. |
| DSC | FIG. 18: Endotherms at about 102° C. and about 266° C. |

Certain XRPD diffraction peaks for the pyridine solvate of the free base of (R)—N-(4-(3-aminopiperidin-1-yl)-5-bromo-1H-pyrrolo[2,3-b]pyridin-3-yl)cyclopropanecarboxamide are shown in Table 19 below.

TABLE 19
SCAN: 2.0/40.0/0.02/0.6 (sec), Cu (30 kV, 15 mA), I (p) = 647.0,
10/25/12 11:01 a
PEAK: 23 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 8.203 | 10.7695 | 11 | 49 | 8.0 | 578 | 6.0 | 0.199 |
| 2 | 9.586 | 9.2188 | 13 | 46 | 7.5 | 597 | 6.2 | 0.219 |
| 3 | 10.839 | 8.1558 | 19 | 540 | 87.3 | 5609 | 57.9 | 0.176 |
| 4 | 12.503 | 7.0742 | 18 | 79 | 12.8 | 1064 | 11.0 | 0.228 |
| 5 | 14.276 | 6.1993 | 21 | 79 | 12.8 | 823 | 8.5 | 0.176 |
| 6 | 16.275 | 5.4418 | 25 | 163 | 26.4 | 2384 | 24.6 | 0.249 |
| 7 | 16.679 | 5.3109 | 28 | 43 | 6.9 | 417 | 4.3 | 0.165 |
| 8 | 18.800 | 4.7162 | 28 | 602 | 97.3 | 7107 | 73.4 | 0.201 |
| 9 | 19.320 | 4.5905 | 28 | 619 | 100.0 | 9214 | 95.1 | 0.253 |
| 10 | 19.621 | 4.5207 | 28 | 468 | 75.6 | 9688 | 100.0 | 0.352 |
| 11 | 20.102 | 4.4138 | 28 | 60 | 9.7 | 1044 | 10.8 | 0.297 |
| 12 | 21.220 | 4.1836 | 52 | 301 | 48.7 | 5459 | 56.3 | 0.308 |
| 13 | 21.682 | 4.0955 | 51 | 457 | 73.8 | 7775 | 80.3 | 0.289 |
| 14 | 22.379 | 3.9695 | 51 | 409 | 66.1 | 6782 | 70.0 | 0.282 |
| 15 | 22.999 | 3.8639 | 51 | 176 | 28.4 | 2551 | 26.3 | 0.247 |
| 16 | 23.479 | 3.7859 | 66 | 138 | 22.3 | 1277 | 13.2 | 0.157 |
| 17 | 24.041 | 3.6987 | 56 | 68 | 11.0 | 648 | 6.7 | 0.162 |
| 18 | 24.416 | 3.6427 | 54 | 74 | 11.9 | 1090 | 11.2 | 0.252 |
| 19 | 24.794 | 3.5880 | 53 | 30 | 4.8 | 358 | 3.7 | 0.203 |
| 20 | 25.262 | 3.5226 | 50 | 48 | 7.8 | 1454 | 15.0 | 0.514 |
| 21 | 25.465 | 3.4950 | 49 | 74 | 12.0 | 1454 | 15.0 | 0.332 |
| 22 | 26.559 | 3.3535 | 43 | 87 | 14.1 | 1883 | 19.4 | 0.367 |
| 23 | 27.006 | 3.2990 | 42 | 70 | 11.4 | 1285 | 13.3 | 0.311 |
| 24 | 28.059 | 3.1775 | 41 | 125 | 20.2 | 1668 | 17.2 | 0.226 |
| 25 | 28.401 | 3.1400 | 41 | 96 | 15.6 | 2265 | 23.4 | 0.400 |
| 26 | 28.858 | 3.0914 | 41 | 136 | 22.0 | 4122 | 42.5 | 0.514 |
| 27 | 29.821 | 2.9936 | 41 | 214 | 34.6 | 2611 | 26.9 | 0.207 |
| 28 | 30.442 | 2.9340 | 41 | 64 | 10.4 | 1514 | 15.6 | 0.400 |
| 29 | 30.799 | 2.9008 | 41 | 93 | 15.1 | 2034 | 21.0 | 0.371 |
| 30 | 31.301 | 2.8554 | 41 | 117 | 19.0 | 3950 | 40.8 | 0.572 |
| 31 | 31.937 | 2.7999 | 41 | 164 | 26.6 | 5554 | 57.3 | 0.575 |
| 32 | 32.740 | 2.7331 | 50 | 105 | 17.0 | 2400 | 24.8 | 0.388 |
| 33 | 34.958 | 2.5646 | 50 | 35 | 5.7 | 616 | 6.4 | 0.297 |
| 34 | 35.657 | 2.5159 | 63 | 62 | 10.0 | 565 | 5.8 | 0.155 |
| 35 | 37.263 | 2.4111 | 61 | 79 | 12.7 | 2159 | 22.3 | 0.466 |
| 36 | 37.521 | 2.3951 | 57 | 76 | 12.4 | 2407 | 24.8 | 0.535 |
| 37 | 38.661 | 2.3271 | 56 | 59 | 9.5 | 365 | 3.8 | 0.106 |
| 38 | 39.316 | 2.2898 | 66 | 78 | 12.7 | 2725 | 28.1 | 0.590 |

### Example 11

(R)—N-(4-(3-aminopiperidin-1-yl)-5-bromo-1H-pyrrolo[2,3-b]pyridin-3-yl)cyclopropanecarboxamide 1,4-dioxane solvate: (R)—N-(4-(3-aminopiperidin-1-yl)-5-bromo-1H-pyrrolo[2,3-b]pyridin-3-yl)cyclopropanecarboxamide (26.1 mg) was dissolved in 1,4-dioxane (1.5 mL) in a 20 mL scintillation vial at room temperature. The solution was filtered and placed into a clean 20 mL scintillation vial. The top was covered tightly with aluminum foil and allowed to evaporate under ambient conditions. The physical properties of the 1,4-dioxane solvate of the free base of (R)—N-(4-(3-aminopiperidin-1-yl)-5-bromo-1H-pyrrolo[2,3-b]pyridin-3-yl)cyclopropanecarboxamide are summarized in Table 20 below.

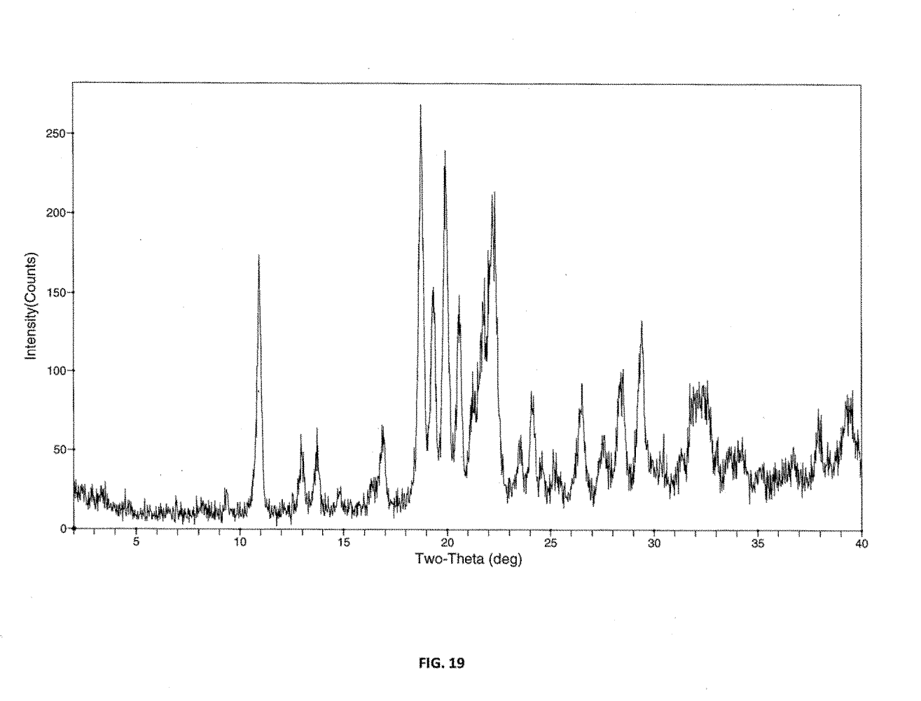

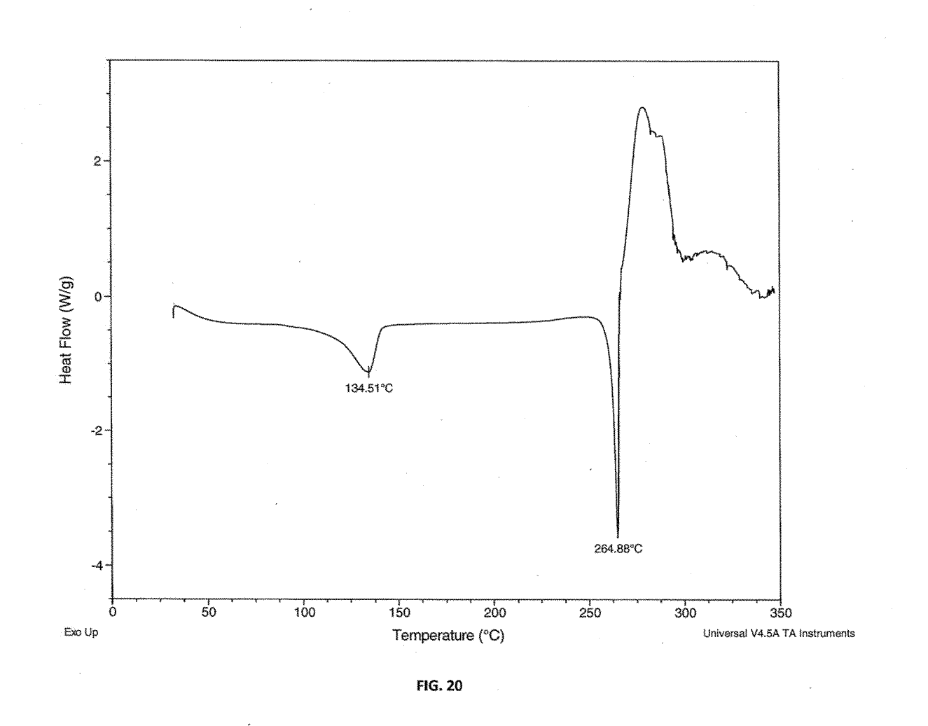

TABLE 20

| | |
|---|---|
| XRPD | FIG. 19: Peaks of ≥25% relative height at about 11.0, 18.8, |
| | 19.4, 19.9, 20.6, 21.3, 21.7, 22.3, 26.5, 28.4, 29.4, and |
| | 31.7 °2θ. |
| DSC | FIG. 20: Endotherms at about 135° C. and about 265° C. |

Certain XRPD diffraction peaks for the 1,4-dioxane solvate of the free base of (R)—N-(4-(3-aminopiperidin-1-yl)-5-bromo-1H-pyrrolo[2,3-b]pyridin-3-yl)cyclopropanecarboxamide are shown in Table 21 below.

TABLE 21
SCAN: 2.0/40.0/0.02/0.6 (sec), Cu (30 kV, 15 mA), I (p) = 269.0,
10/29/12 12:11 p
PEAK: 27 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 10.960 | 8.0663 | 11 | 163 | 64.8 | 2125 | 32.9 | 0.221 |
| 2 | 12.944 | 6.8338 | 11 | 49 | 19.3 | 681 | 10.5 | 0.238 |
| 3 | 13.723 | 6.4478 | 18 | 46 | 18.2 | 273 | 4.2 | 0.101 |
| 4 | 16.860 | 5.2543 | 15 | 51 | 20.3 | 1010 | 15.6 | 0.335 |
| 5 | 18.780 | 4.7212 | 17 | 252 | 100.0 | 4273 | 66.2 | 0.288 |
| 6 | 19.359 | 4.5814 | 17 | 137 | 54.4 | 2719 | 42.1 | 0.337 |
| 7 | 19.940 | 4.4492 | 17 | 223 | 88.5 | 3959 | 61.3 | 0.301 |
| 8 | 20.600 | 4.3082 | 17 | 132 | 52.4 | 1977 | 30.6 | 0.254 |
| 9 | 21.261 | 4.1757 | 17 | 83 | 33.0 | 1243 | 19.3 | 0.254 |
| 10 | 21.743 | 4.0840 | 17 | 128 | 50.8 | 6259 | 96.9 | 0.829 |
| 11 | 22.340 | 3.9763 | 24 | 190 | 75.3 | 6457 | 100.0 | 0.578 |
| 12 | 23.557 | 3.7736 | 27 | 33 | 13.0 | 486 | 7.5 | 0.252 |
| 13 | 24.080 | 3.6927 | 26 | 62 | 24.4 | 991 | 15.3 | 0.274 |
| 14 | 25.145 | 3.5388 | 24 | 27 | 10.7 | 257 | 4.0 | 0.162 |
| 15 | 26.521 | 3.3581 | 22 | 71 | 28.1 | 1232 | 19.1 | 0.296 |
| 16 | 27.503 | 3.2405 | 22 | 38 | 15.2 | 696 | 10.8 | 0.309 |
| 17 | 28.417 | 3.1383 | 22 | 78 | 31.0 | 1843 | 28.5 | 0.400 |
| 18 | 29.420 | 3.0335 | 22 | 111 | 44.1 | 2614 | 40.5 | 0.399 |
| 19 | 31.744 | 2.8165 | 28 | 65 | 25.7 | 3183 | 49.3 | 0.834 |
| 20 | 32.419 | 2.7595 | 38 | 54 | 21.6 | 2453 | 38.0 | 0.766 |
| 21 | 34.278 | 2.6139 | 32 | 27 | 10.9 | 666 | 10.3 | 0.413 |
| 22 | 37.916 | 2.3710 | 37 | 40 | 16.0 | 391 | 6.1 | 0.165 |
| 23 | 39.314 | 2.2899 | 42 | 44 | 17.5 | 1121 | 17.4 | 0.431 |

### Example 12

(R)—N-(4-(3-aminopiperidin-1-yl)-5-bromo-1H-pyrrolo[2,3-b]pyridin-3-yl)cyclopropanecarboxamide 2-butanol solvate: 2-butanol (1.5 mL) and heptane (7.5 mL) were added to (R)—N-(4-(3-aminopiperidin-1-yl)-5-bromo-1H-pyrrolo[2,3-b]pyridin-3-yl)cyclopropanecarboxamide (25.9 mg). The mixture was placed in the refrigerator at approximately 5° C. for 5 days. Solvent was removed from the suspension while still cold and the material analyzed while still damp with solvent. The physical properties of the 2-butanol solvate of the free base of (R)—N-(4-(3-aminopiperidin-1-yl)-5-bromo-1H-pyrrolo[2,3-b]pyridin-3-yl)cyclopropanecarboxamide are summarized in Table 22 below.

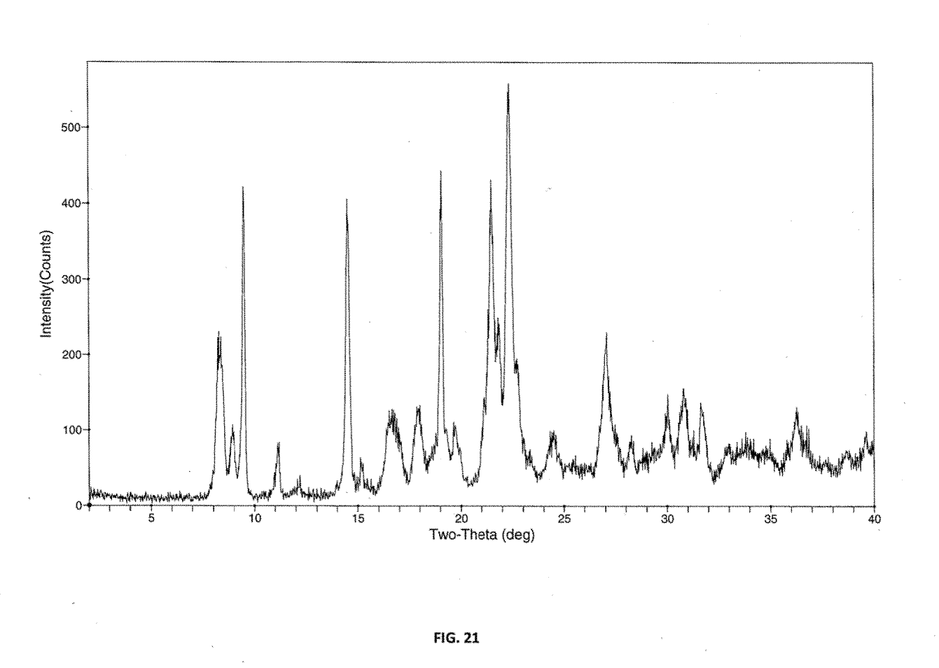

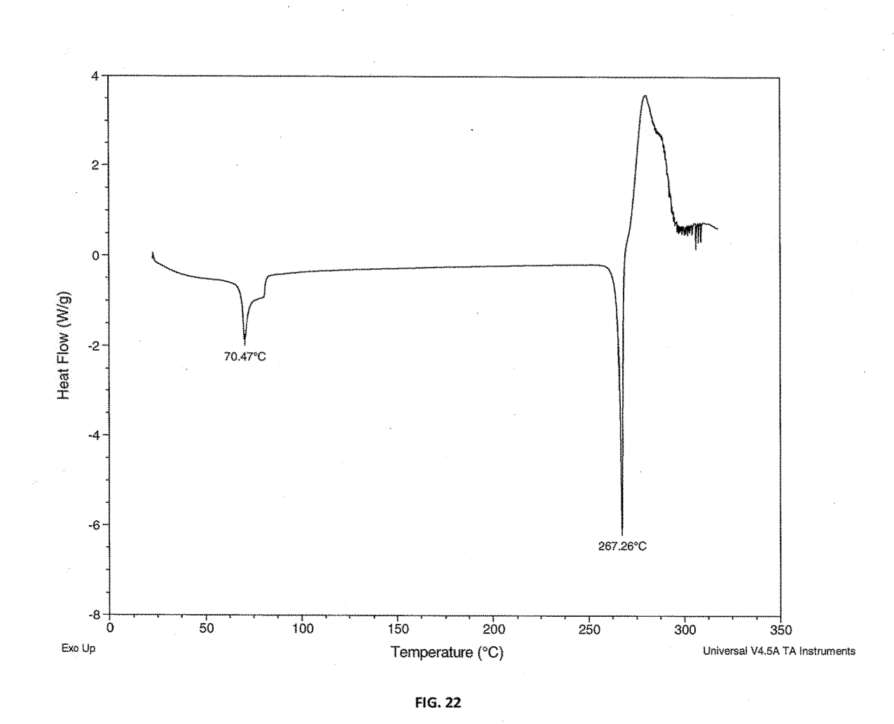

TABLE 22

| | |
|---|---|
| XRPD | FIG. 21: Peaks of ≥20% relative height at about 8.3, 9.5, |
| | 14.5, 16.5, 18.0, 19.1, 21.5, 22.4, and 27.1 °2θ. |
| DSC | FIG. 22: Endotherms at about 70° C. and about 267° C. |

Certain XRPD diffraction peaks for the 2-butanol solvate of the free base of (R)—N-(4-(3-aminopiperidin-1-yl)-5-bromo-1H-pyrrolo[2,3-b]pyridin-3-yl)cyclopropanecarboxamide are shown in Table 23 below.

TABLE 23
SCAN: 2.0/40.0/0.02/0.6 (sec), Cu (30 kV, 15 mA), I (p) = 559.0,
11/19/12 10:19 a
PEAK: 27 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 8.279 | 10.6709 | 10 | 221 | 41.9 | 4905 | 42.6 | 0.377 |
| 2 | 8.940 | 9.8841 | 11 | 96 | 18.1 | 2073 | 18.0 | 0.369 |
| 3 | 9.502 | 9.3004 | 11 | 411 | 77.8 | 3632 | 31.6 | 0.150 |
| 4 | 11.193 | 7.8985 | 15 | 69 | 13.2 | 728 | 6.3 | 0.178 |
| 5 | 14.521 | 6.0948 | 13 | 393 | 74.6 | 5025 | 43.7 | 0.217 |
| 6 | 15.148 | 5.8440 | 13 | 51 | 9.7 | 861 | 7.5 | 0.285 |
| 7 | 16.520 | 5.3619 | 19 | 107 | 20.4 | 4078 | 35.4 | 0.645 |
| 8 | 18.018 | 4.9193 | 19 | 114 | 21.7 | 2948 | 25.6 | 0.438 |
| 9 | 19.078 | 4.6481 | 19 | 424 | 80.4 | 6836 | 59.4 | 0.274 |
| 10 | 19.721 | 4.4981 | 19 | 88 | 16.8 | 2054 | 17.8 | 0.395 |
| 11 | 21.500 | 4.1297 | 31 | 401 | 75.9 | 10031 | 87.2 | 0.426 |
| 12 | 22.379 | 3.9694 | 31 | 528 | 100.0 | 11508 | 100.0 | 0.371 |
| 13 | 24.518 | 3.6278 | 44 | 56 | 10.6 | 1242 | 10.8 | 0.378 |
| 14 | 27.078 | 3.2904 | 47 | 183 | 34.8 | 4405 | 38.3 | 0.408 |
| 15 | 28.279 | 3.1533 | 50 | 44 | 8.3 | 444 | 3.9 | 0.173 |
| 16 | 30.042 | 2.9721 | 67 | 81 | 15.4 | 1008 | 8.8 | 0.211 |
| 17 | 30.837 | 2.8973 | 69 | 87 | 16.5 | 1578 | 13.7 | 0.308 |
| 18 | 31.678 | 2.8222 | 38 | 92 | 17.4 | 1802 | 15.7 | 0.333 |
| 19 | 32.979 | 2.7138 | 38 | 43 | 8.2 | 1141 | 9.9 | 0.451 |
| 20 | 33.903 | 2.6419 | 38 | 53 | 10.0 | 1919 | 16.7 | 0.616 |
| 21 | 34.779 | 2.5774 | 38 | 42 | 8.0 | 1380 | 12.0 | 0.559 |
| 22 | 34.981 | 2.5629 | 38 | 51 | 9.7 | 1380 | 12.0 | 0.460 |
| 23 | 36.278 | 2.4743 | 54 | 77 | 14.5 | 2024 | 17.6 | 0.449 |

### Example 13 (R)—N-(4-(3-aminopiperidin-1-yl)-5-bromo-1H-pyrrolo[2,3-b]pyridin-3-yl)cyclopropanecarboxamide anisole solvate

Method A: (R)—N-(4-(3-aminopiperidin-1-yl)-5-bromo-1H-pyrrolo[2,3-b]pyridin-3-yl)cyclopropanecarboxamide (27.6 mg) was dissolved in anisole (4 mL) at approximately 60° C. The solution was removed from the cooling block and allowed to cool to ambient. The vial was then placed into a freezer at about 18° C. and left there for 3 weeks. The solvent was removed while the sample was still cold and the solid analyzed while still damp with solvent.

Method B: (R)—N-(4-(3-aminopiperidin-1-yl)-5-bromo-1H-pyrrolo[2,3-b]pyridin-3-yl)cyclopropanecarboxamide was dissolved at 25 mg/mL in anisole 65° C., and cooled from 65° C. to 10° C. over 14.5 h. Solids were isolated via filtration to yield (R)—N-(4-(3-aminopiperidin-1-yl)-5-bromo-1H-pyrrolo[2,3-b]pyridin-3-yl)cyclopropanecarboxamide anisole solvate.

The physical properties of the anisole solvate of the free base of (R)—N-(4-(3-aminopiperidin-1-yl)-5-bromo-1H-pyrrolo[2,3-b]pyridin-3-yl)cyclopropanecarboxamide are summarized in Table 24 below.

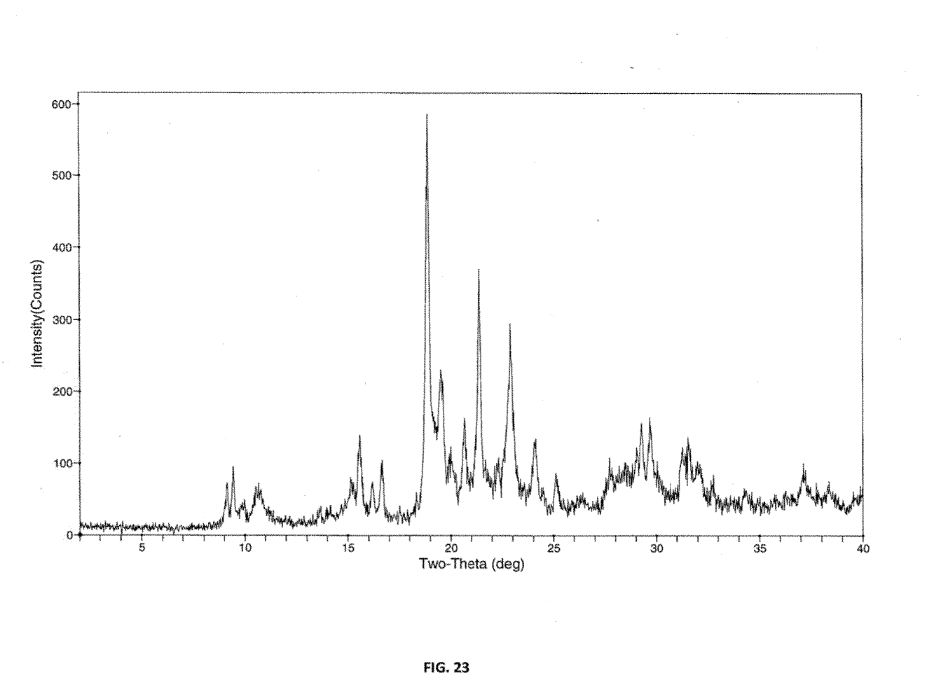

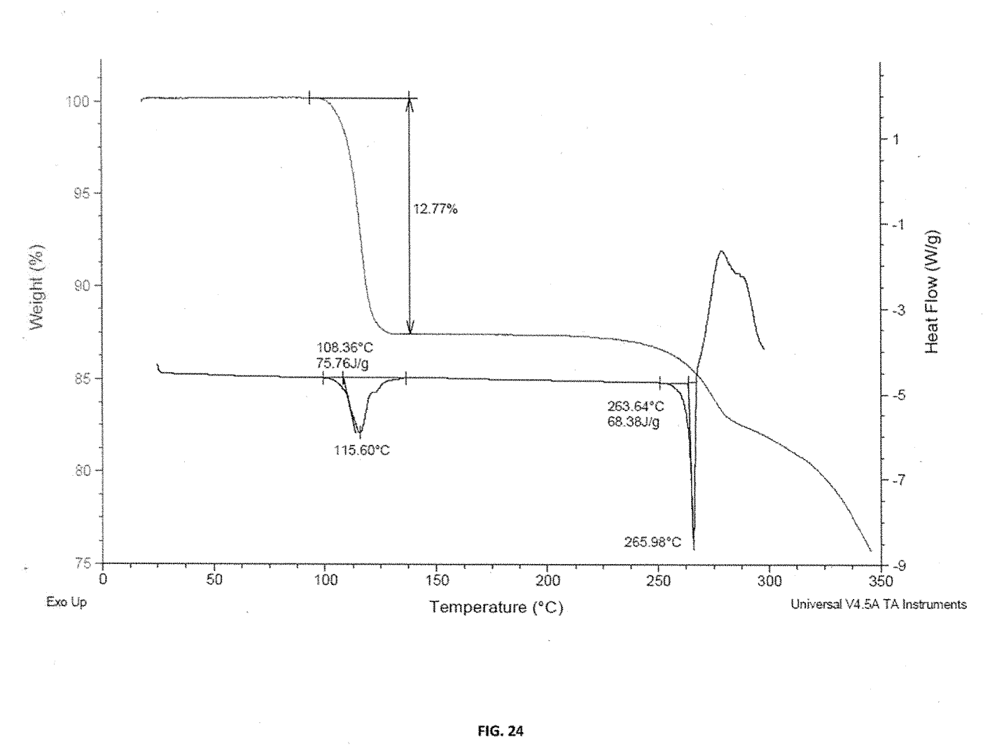

TABLE 24

| | |
|---|---|
| XRPD | FIG. 23: Peaks of ≥15% relative height at about 15.6, 18.9, |
| | 19.5, 20.0, 20.7, 21.4, 22.9, 24.1, and 29.7 °2θ. |
| DSC | FIG. 24: Endotherm with extrapolated onset temperature about |
| | 108° C. and enthalpy of fusion about 76 J/g. Endotherm with |
| | extrapolated onset temperature about 264° C. and enthalpy of |
| | fusion about 68 J/g. |
| TGA | FIG. 24: About 13.0% weight loss up to about 140° C. |

Certain XRPD diffraction peaks for the anisole solvate of the free base of (R)—N-(4-(3-aminopiperidin-1-yl)-5-bromo-1H-pyrrolo[2,3-b]pyridin-3-yl)cyclopropanecarboxamide are shown in Table 25 below.

TABLE 25
SCAN: 2.0/40.0/0.02/0.6 (sec), Cu (30 kV, 15 mA), I (p) = 587.0,
12/03/12 02:55 p
PEAK: 27 (pts)/Parabolic Filter, Threshold = 3.0, Cutoff = 0.1%,
BG = 3/1.0, Peak-Top = Summit
NOTE: Intensity = Counts, 2T (0) = 0.0 (deg), Wavelength to Compute
d-Spacing = 1.54059 Å (Cu/K-alpha1)

| # | 2-Theta | d (Å) | BG | Height | H % | Area | A % | FWHM |
|---|---|---|---|---|---|---|---|---|
| 1 | 9.156 | 9.6509 | 11 | 62 | 11.0 | 1049 | 10.7 | 0.287 |
| 2 | 9.441 | 9.3600 | 15 | 81 | 14.4 | 1722 | 17.5 | 0.362 |
| 3 | 9.978 | 8.8576 | 15 | 35 | 6.2 | 428 | 4.4 | 0.209 |
| 4 | 10.678 | 8.2788 | 30 | 43 | 7.6 | 627 | 6.4 | 0.249 |
| 5 | 15.144 | 5.8456 | 31 | 50 | 8.9 | 1000 | 10.2 | 0.338 |
| 6 | 15.596 | 5.6771 | 35 | 105 | 18.7 | 1427 | 14.5 | 0.230 |
| 7 | 16.197 | 5.4678 | 37 | 38 | 6.8 | 314 | 3.2 | 0.139 |
| 8 | 16.658 | 5.3177 | 31 | 74 | 13.2 | 680 | 6.9 | 0.156 |
| 9 | 18.335 | 4.8349 | 25 | 23 | 4.1 | 287 | 2.9 | 0.212 |
| 10 | 18.919 | 4.6869 | 24 | 563 | 100.0 | 9835 | 100.0 | 0.297 |
| 11 | 19.520 | 4.5438 | 24 | 207 | 36.7 | 7786 | 79.2 | 0.640 |
| 12 | 20.002 | 4.4355 | 24 | 100 | 17.7 | 1811 | 18.4 | 0.309 |
| 13 | 20.662 | 4.2953 | 24 | 139 | 24.7 | 2126 | 21.6 | 0.261 |
| 14 | 21.400 | 4.1487 | 24 | 347 | 61.6 | 6203 | 63.1 | 0.304 |
| 15 | 22.243 | 3.9935 | 24 | 77 | 13.6 | 1200 | 12.2 | 0.266 |
| 16 | 22.901 | 3.8802 | 24 | 272 | 48.3 | 6568 | 66.8 | 0.411 |
| 17 | 24.118 | 3.6870 | 39 | 96 | 17.1 | 1747 | 17.8 | 0.309 |
| 18 | 24.503 | 3.6300 | 39 | 28 | 5.0 | 490 | 5.0 | 0.296 |
| 19 | 25.120 | 3.5422 | 38 | 49 | 8.7 | 505 | 5.1 | 0.175 |
| 20 | 27.720 | 3.2156 | 40 | 69 | 12.3 | 930 | 9.5 | 0.228 |
| 21 | 29.280 | 3.0477 | 77 | 80 | 14.1 | 1062 | 10.8 | 0.227 |
| 22 | 29.684 | 3.0071 | 69 | 95 | 16.8 | 1621 | 16.5 | 0.291 |
| 23 | 31.277 | 2.8575 | 62 | 61 | 10.8 | 663 | 6.7 | 0.185 |
| 24 | 31.550 | 2.8334 | 54 | 83 | 14.8 | 2800 | 28.5 | 0.572 |
| 25 | 32.001 | 2.7945 | 57 | 47 | 8.4 | 1348 | 13.7 | 0.486 |
| 26 | 34.279 | 2.6139 | 38 | 28 | 4.9 | 404 | 4.1 | 0.248 |
| 27 | 37.140 | 2.4188 | 51 | 50 | 8.8 | 566 | 5.8 | 0.194 |

### Example 14

(R)—N-(4-(3-aminopiperidin-1-yl)-5-bromo-1H-pyrrolo[2,3-b]pyridin-3-yl)cyclopropanecarboxamide 1-propanol solvate: (R)—N-(4-(3-aminopiperidin-1-yl)-5-bromo-1H-pyrrolo[2,3-b]pyridin-3-yl)cyclopropanecarboxamide was suspended at 60° C. in heptane (1 mL). 1-Propanol (2 mL) was added. The solids dissolved. The solution allowed to cool to ambient. The vial was then placed into a freezer at about −18° C. and left there for 3 weeks. The solvent was removed while the sample was still cold and the solid analyzed while still damp with solvent. The physical properties of the 1-propanol solvate of the free base of (R)—N-(4-(3-aminopiperidin-1-yl)-5-bromo-1H-pyrrolo[2,3-b]pyridin-3-yl)cyclopropanecarboxamide are summarized in Table 26 below.

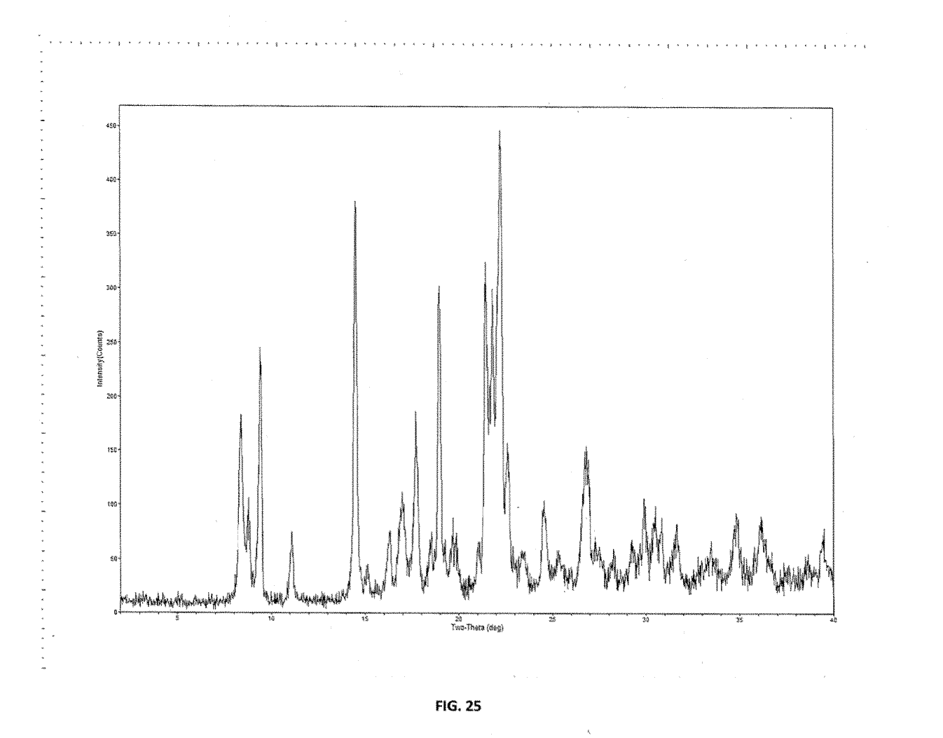

TABLE 26

| | |
|---|---|
| XRPD | FIG. 25: Peaks of ≥20% relative height at about 8.4, 9.4, 14.5, |
| | 17.7, 19.0, 21.5, 21.8, 22.3, 22.6, and 26.8 °2θ. |

Certain XRPD diffraction peaks for the 1-propanol solvate of the free base of (R)—N-(4-(3-aminopiperidin-1-yl)-5-bromo-1H-pyrrolo[2,3-b]pyridin-3-yl)cyclopropanecarboxamide are shown in Table 27 below.

TABLE 27
SCAN: 2.0/40.0/0.02/0.6(sec), Cu(30 kV, 15 mA),
I(p) = 446.0, 12/18/12 09:44a
PEAK: 21(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 8.438 | 10.4709 | 11 | 172 | 41.3 | 3015 | 35.4 | 0.297 |
| 2 | 8.822 | 10.0153 | 14 | 92 | 22.1 | 1915 | 22.5 | 0.353 |
| 3 | 9.441 | 9.3603 | 12 | 233 | 55.7 | 2482 | 29.1 | 0.181 |
| 4 | 11.138 | 7.9373 | 12 | 63 | 15.2 | 697 | 8.2 | 0.187 |
| 5 | 14.539 | 6.0877 | 11 | 369 | 88.2 | 4456 | 52.3 | 0.206 |
| 6 | 15.179 | 5.8323 | 11 | 34 | 8.0 | 588 | 6.9 | 0.298 |
| 7 | 16.301 | 5.4333 | 19 | 56 | 13.3 | 792 | 9.3 | 0.242 |
| 8 | 16.998 | 5.2119 | 28 | 84 | 20.0 | 1792 | 21.0 | 0.364 |
| 9 | 17.740 | 4.9955 | 26 | 160 | 38.3 | 2012 | 23.6 | 0.214 |
| 10 | 18.580 | 4.7717 | 30 | 45 | 10.7 | 598 | 7.0 | 0.227 |
| 11 | 19.000 | 4.6672 | 34 | 268 | 64.1 | 3122 | 36.6 | 0.198 |
| 12 | 21.079 | 4.2112 | 24 | 42 | 10.1 | 498 | 5.8 | 0.201 |
| 13 | 21.462 | 4.1370 | 27 | 297 | 71.0 | 6018 | 70.6 | 0.345 |
| 14 | 21.842 | 4.0658 | 28 | 271 | 64.8 | 6637 | 77.9 | 0.417 |
| 15 | 22.261 | 3.9903 | 28 | 418 | 100.0 | 8520 | 100.0 | 0.347 |
| 16 | 22.641 | 3.9242 | 41 | 116 | 27.9 | 1302 | 15.3 | 0.190 |
| 17 | 24.561 | 3.6216 | 34 | 70 | 16.7 | 778 | 9.1 | 0.189 |
| 18 | 25.357 | 3.5096 | 33 | 25 | 6.0 | 588 | 6.9 | 0.402 |
| 19 | 26.841 | 3.3189 | 33 | 121 | 29.0 | 2977 | 34.9 | 0.417 |
| 20 | 27.592 | 3.2302 | 29 | 27 | 6.6 | 420 | 4.9 | 0.261 |
| 21 | 29.279 | 3.0478 | 31 | 37 | 8.9 | 803 | 9.4 | 0.368 |
| 22 | 29.902 | 2.9857 | 32 | 74 | 17.7 | 1288 | 15.1 | 0.295 |
| 23 | 30.519 | 2.9267 | 46 | 53 | 12.7 | 803 | 9.4 | 0.256 |
| 24 | 30.873 | 2.8940 | 43 | 45 | 10.7 | 439 | 5.2 | 0.167 |
| 25 | 31.678 | 2.8223 | 27 | 56 | 13.5 | 992 | 11.6 | 0.299 |
| 26 | 33.500 | 2.6728 | 34 | 33 | 8.0 | 674 | 7.9 | 0.343 |
| 27 | 34.864 | 2.5713 | 37 | 56 | 13.5 | 891 | 10.5 | 0.269 |
| 28 | 35.827 | 2.5044 | 37 | 25 | 5.9 | 153 | 1.8 | 0.106 |
| 29 | 36.161 | 2.4820 | 32 | 53 | 12.6 | 1338 | 15.7 | 0.433 |
| 30 | 39.442 | 2.2828 | 35 | 33 | 7.9 | 495 | 5.8 | 0.254 |

NOTE:
Intensity = Counts, 2T(0) = 0.0(deg), Wavelength to Compute d-Spacing = 1.54059 Å (Cu/K-alpha1)

### Example 15 (R)—N-(4-(3-aminopiperidin-1-yl)-5-bromo-1H-pyrrolo[2,3-b]pyridin-3-yl)cyclopropanecarboxamide bis-ethanol solvate

Method A: A saturated solution of (R)—N-(4-(3-aminopiperidin-1-yl)-5-bromo-1H-pyrrolo[2,3-b]pyridin-3-yl)cyclopropanecarboxamide was generated at 65° C. without stirring and the sample was held at 65° C. for 24 hours. Solid consisting of plates were observed above the liquid level. The solution was cooled to room temperature.

Method B: A saturated solution of (R)—N-(4-(3-aminopiperidin-1-yl)-5-bromo-1H-pyrrolo[2,3-b]pyridin-3-yl)cyclopropanecarboxamide was generated in ethanol at room temperature. The solution was subjected to vapor diffusion using water and isooctane. The vial within vials were placed in a refrigerator at about 5-10° C. to give some plates of the ethanol solvate.

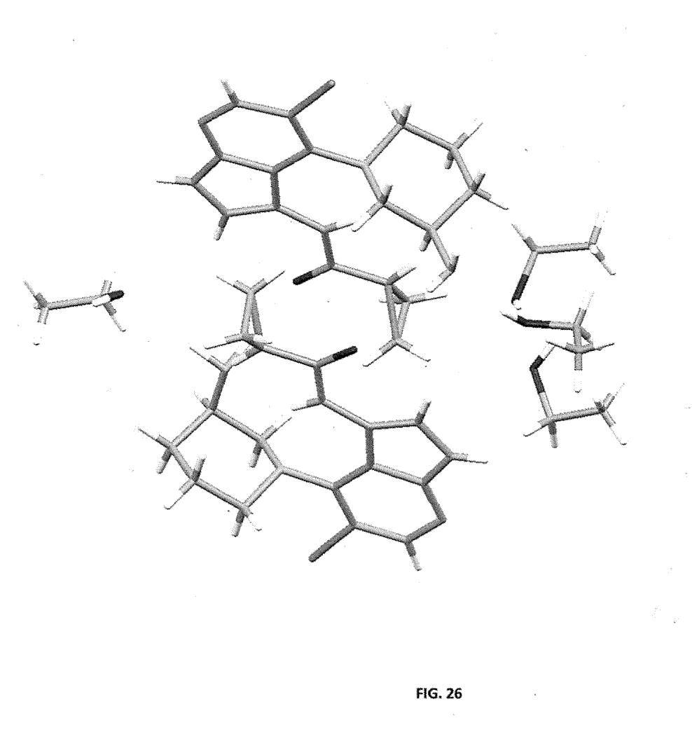

A yellow plate 0.050×0.040×0.030 mm in size was mounted on a Cryoloop with Paratone oil. Data were collected in a nitrogen gas stream at 100(2) K using and scans. Crystal-to-detector distance was 60 mm and exposure time was 5 seconds per frame using a scan width of 2.0°. Data collection was 98.4% complete to 67.000° in q. A total of 32,872 reflections were collected covering the indices, −11<=h<=11, −11<=k<=11, −15<=l<=15. 7,254 reflections were found to be symmetry independent, with an Rᵢₙₜ of 0.0248. Indexing and unit cell refinement indicated a primitive, triclinic lattice. The space group was found to be P 1 (No. 1). The data were integrated using the Bruker SAINT software program and scaled using the SADABS software program. Solution by iterative methods (SHELXT-2014) produced a complete heavy-atom phasing model consistent with the proposed structure. All non-hydrogen atoms were refined anisotropically by full-matrix least-squares (SHELXL-2014). All hydrogen atoms were placed using a riding model. Their positions were constrained relative to their parent atom using the appropriate HFIX command in SHELXL-2014. The crystal structure of the asymmetric unit for the bis-ethanol solvate is shown in FIG. 26.

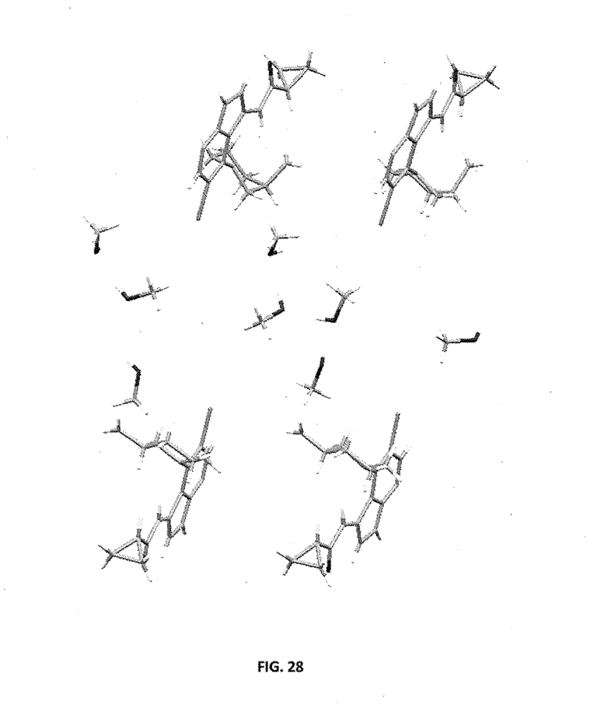

The XRPD of the bis-ethanol solvate of the free base of (R)—N-(4-(3-aminopiperidin-1-yl)-5-bromo-1H-pyrrolo[2,3-b]pyridin-3-yl)cyclopropanecarboxamide, as calculated from the SCXRD data at 100 K, is shown in FIG. 28 and summarized in Table 28 below.

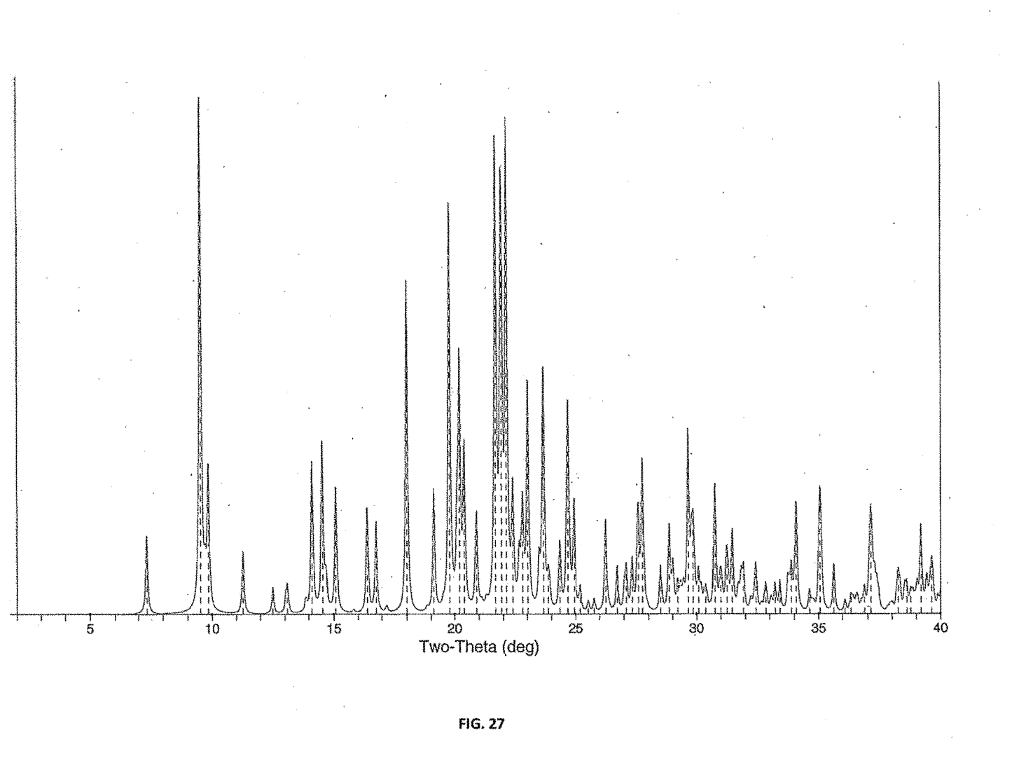

TABLE 28

| | |
|---|---|
| XRPD | FIG. 27: Peaks of ≥30% relative height at about 9.5, 14.6, |
| | 18.0, 19.8, 20.2, 20.4, 21.7, 21.9, 22.1, 23.0, 23.7, 24.7, |
| | and 29.7 °2θ. |

Certain calculated XRPD diffraction peaks for the bis-ethanol solvate of the free base of (R)—N-(4-(3-aminopiperidin-1-yl)-5-bromo-1H-pyrrolo[2,3-b]pyridin-3-yl)cyclopropanecarboxamide are shown in Table 29 below.

TABLE 29
SCAN: 2.0/40.0/0.02/0.1(sec), Cu, I(p) = 10000, 02/05/16 11:12a
PEAK: 15(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 7.338 | 12.0378 | 8 | 1491 | 15.0 | 10309 | 13.2 | 0.118 |
| 2 | 9.537 | 9.2664 | 32 | 9968 | 100.0 | 78258 | 100.0 | 0.133 |
| 3 | 9.858 | 8.9651 | 31 | 2871 | 28.8 | 34398 | 44.0 | 0.204 |
| 4 | 11.280 | 7.8380 | 10 | 1190 | 11.9 | 8194 | 10.5 | 0.117 |
| 5 | 12.520 | 7.0642 | 10 | 498 | 5.0 | 3572 | 4.6 | 0.122 |
| 6 | 13.098 | 6.7536 | 11 | 584 | 5.9 | 5617 | 7.2 | 0.163 |
| 7 | 14.139 | 6.2588 | 35 | 2907 | 29.2 | 23604 | 30.2 | 0.138 |
| 8 | 14.557 | 6.0800 | 40 | 3315 | 33.3 | 29799 | 38.1 | 0.153 |
| 9 | 15.083 | 5.8691 | 39 | 2409 | 24.2 | 16922 | 21.6 | 0.119 |
| 10 | 16.383 | 5.4063 | 47 | 2000 | 20.1 | 14372 | 18.4 | 0.122 |
| 11 | 16.758 | 5.2860 | 53 | 1729 | 17.3 | 12328 | 15.8 | 0.121 |
| 12 | 18.000 | 4.9241 | 64 | 6375 | 64.0 | 42365 | 54.1 | 0.113 |
| 13 | 19.121 | 4.6378 | 71 | 2340 | 23.5 | 17156 | 21.9 | 0.125 |
| 14 | 19.783 | 4.4841 | 71 | 7857 | 78.8 | 62870 | 80.3 | 0.136 |
| 15 | 20.201 | 4.3923 | 71 | 5057 | 50.7 | 55380 | 70.8 | 0.186 |
| 16 | 20.399 | 4.3501 | 71 | 3309 | 33.2 | 38500 | 49.2 | 0.198 |
| 17 | 20.897 | 4.2475 | 71 | 1902 | 19.1 | 15605 | 19.9 | 0.139 |
| 18 | 21.683 | 4.0953 | 277 | 8971 | 90.0 | 75923 | 97.0 | 0.144 |
| 19 | 21.921 | 4.0514 | 277 | 8357 | 83.8 | 74257 | 94.9 | 0.151 |
| 20 | 22.141 | 4.0117 | 277 | 9328 | 93.6 | 77230 | 98.7 | 0.141 |
| 21 | 22.402 | 3.9655 | 277 | 2359 | 23.7 | 23831 | 30.5 | 0.172 |
| 22 | 22.820 | 3.8938 | 320 | 2037 | 20.4 | 29004 | 37.1 | 0.242 |
| 23 | 23.023 | 3.8598 | 251 | 4270 | 42.8 | 32690 | 41.8 | 0.130 |
| 24 | 23.663 | 3.7569 | 195 | 4577 | 45.9 | 40247 | 51.4 | 0.149 |
| 25 | 23.860 | 3.7263 | 195 | 757 | 7.6 | 15074 | 19.3 | 0.338 |
| 26 | 24.343 | 3.6535 | 109 | 1296 | 13.0 | 10236 | 13.1 | 0.134 |
| 27 | 24.679 | 3.6045 | 111 | 4026 | 40.4 | 34332 | 43.9 | 0.145 |
| 28 | 24.937 | 3.5677 | 88 | 2133 | 21.4 | 18544 | 23.7 | 0.148 |
| 29 | 25.197 | 3.5316 | 88 | 472 | 4.7 | 5059 | 6.5 | 0.182 |
| 30 | 25.539 | 3.4850 | 98 | 167 | 1.7 | 780 | 1.0 | 0.079 |
| 31 | 25.785 | 3.4524 | 101 | 198 | 2.0 | 864 | 1.1 | 0.074 |
| 32 | 26.257 | 3.3913 | 88 | 1728 | 17.3 | 11091 | 14.2 | 0.109 |
| 33 | 26.717 | 3.3340 | 88 | 839 | 8.4 | 5522 | 7.1 | 0.112 |
| 34 | 27.096 | 3.2882 | 125 | 851 | 8.5 | 8269 | 10.6 | 0.165 |
| 35 | 27.340 | 3.2594 | 85 | 1009 | 10.1 | 8744 | 11.2 | 0.147 |
| 36 | 27.598 | 3.2296 | 85 | 2054 | 20.6 | 21762 | 27.8 | 0.180 |
| 37 | 27.762 | 3.2108 | 85 | 2922 | 29.3 | 28601 | 36.5 | 0.166 |
| 38 | 28.520 | 3.1272 | 85 | 843 | 8.5 | 5226 | 6.7 | 0.105 |
| 39 | 28.862 | 3.0909 | 85 | 1652 | 16.6 | 13348 | 17.1 | 0.137 |
| 40 | 29.219 | 3.0540 | 85 | 600 | 6.0 | 8712 | 11.1 | 0.247 |
| 41 | 29.662 | 3.0093 | 85 | 3488 | 35.0 | 48456 | 61.9 | 0.236 |
| 42 | 29.858 | 2.9900 | 85 | 1934 | 19.4 | 37292 | 47.7 | 0.328 |
| 43 | 30.080 | 2.9684 | 85 | 830 | 8.3 | 15575 | 19.9 | 0.319 |
| 44 | 30.379 | 2.9399 | 85 | 490 | 4.9 | 5353 | 6.8 | 0.186 |
| 45 | 30.777 | 2.9028 | 85 | 2419 | 24.3 | 17572 | 22.5 | 0.124 |
| 46 | 31.001 | 2.8823 | 85 | 817 | 8.2 | 11227 | 14.3 | 0.234 |
| 47 | 31.260 | 2.8591 | 85 | 1231 | 12.4 | 13104 | 16.7 | 0.181 |
| 48 | 31.479 | 2.8396 | 85 | 1544 | 15.5 | 13371 | 17.1 | 0.147 |
| 49 | 31.937 | 2.7999 | 85 | 909 | 9.1 | 12362 | 15.8 | 0.231 |
| 50 | 32.439 | 2.7577 | 85 | 898 | 9.0 | 8322 | 10.6 | 0.158 |
| 51 | 32.840 | 2.7250 | 85 | 531 | 5.3 | 6016 | 7.7 | 0.193 |
| 52 | 33.222 | 2.6945 | 85 | 520 | 5.2 | 7051 | 9.0 | 0.230 |
| 53 | 33.420 | 2.6791 | 100 | 546 | 5.5 | 4272 | 5.5 | 0.133 |
| 54 | 33.880 | 2.6437 | 100 | 927 | 9.3 | 18600 | 23.8 | 0.341 |
| 55 | 34.083 | 2.6284 | 100 | 2063 | 20.7 | 17666 | 22.6 | 0.146 |
| 56 | 34.640 | 2.5874 | 102 | 379 | 3.8 | 4042 | 5.2 | 0.181 |
| 57 | 35.062 | 2.5572 | 87 | 2365 | 23.7 | 21954 | 28.1 | 0.158 |
| 58 | 35.642 | 2.5169 | 68 | 884 | 8.9 | 5669 | 7.2 | 0.109 |
| 59 | 36.101 | 2.4860 | 68 | 211 | 2.1 | 1117 | 1.4 | 0.090 |
| 60 | 36.342 | 2.4700 | 68 | 329 | 3.3 | 5650 | 7.2 | 0.292 |
| 61 | 36.879 | 2.4353 | 79 | 484 | 4.9 | 8151 | 10.4 | 0.286 |
| 62 | 37.140 | 2.4188 | 88 | 2015 | 20.2 | 30446 | 38.9 | 0.257 |
| 63 | 38.262 | 2.3504 | 88 | 793 | 8.0 | 9301 | 11.9 | 0.199 |
| 64 | 38.617 | 2.3296 | 88 | 573 | 5.7 | 6454 | 8.2 | 0.192 |
| 65 | 38.782 | 2.3201 | 108 | 407 | 4.1 | 15742 | 20.1 | 0.658 |
| 66 | 39.201 | 2.2963 | 108 | 1616 | 16.2 | 25916 | 33.1 | 0.273 |
| 67 | 39.640 | 2.2718 | 108 | 995 | 10.0 | 11361 | 14.5 | 0.194 |

NOTE:
Intensity = Counts, 2T(0) = 0.0(deg), Wavelength to Compute d-Spacing = 1.54059 Å (Cu/K-alpha1)

### Example 16

(R)—N-(4-(3-aminopiperidin-1-yl)-5-bromo-1H-pyrrolo[2,3-b]pyridin-3-yl)cyclopropanecarboxamide bis-methanol solvate: X-ray quality crystals were grown from a saturated, heated methanol solution of (R)—N-(4-(3-aminopiperidin-1-yl)-5-bromo-1H-pyrrolo[2,3-b]pyridin-3-yl)cyclopropanecarboxamide followed by the slow cooling and evaporation of the solvent overnight and allowing the super saturated solution to stand for 2 months to give colorless plates.

A colorless plate 0.060×0.040×0.020 mm in size was mounted on a Cryoloop with Paratone oil. Data were collected in a nitrogen gas stream at 100(2) K using phi and omega scans. Crystal-to-detector distance was 60 mm and exposure time was 10 seconds per frame using a scan width of 1.0°. Data collection was 100.0% complete to 25.000° in q. A total of 39,817 reflections were collected covering the indices, −11<=h<=11, −15<=k<=15, −21<=l<=21. 14799 reflections were found to be symmetry independent, with an Rᵢₙₜ of 0.0383. Indexing and unit cell refinement indicated a primitive, triclinic lattice. The space group was found to be P 1 (No. 1). The data were integrated using the Bruker SAINT software program and scaled using the SADABS software program. Solution by iterative methods (SHELXT) produced a complete heavy-atom phasing model consistent with the proposed structure. All non-hydrogen atoms were refined anisotropically by full-matrix least-squares (SHELXL-2014). All hydrogen atoms were placed using a riding model. Their positions were constrained relative to their parent atom using the appropriate HFIX command in SHELXL-2014. Absolute stereochemistry was unambiguously determined to be R at C15, C31, C47, and C63, respectively. The crystal structure of the asymmetric unit for the bis-methanol solvate along the crystallographic b axis is shown in FIG. 28.

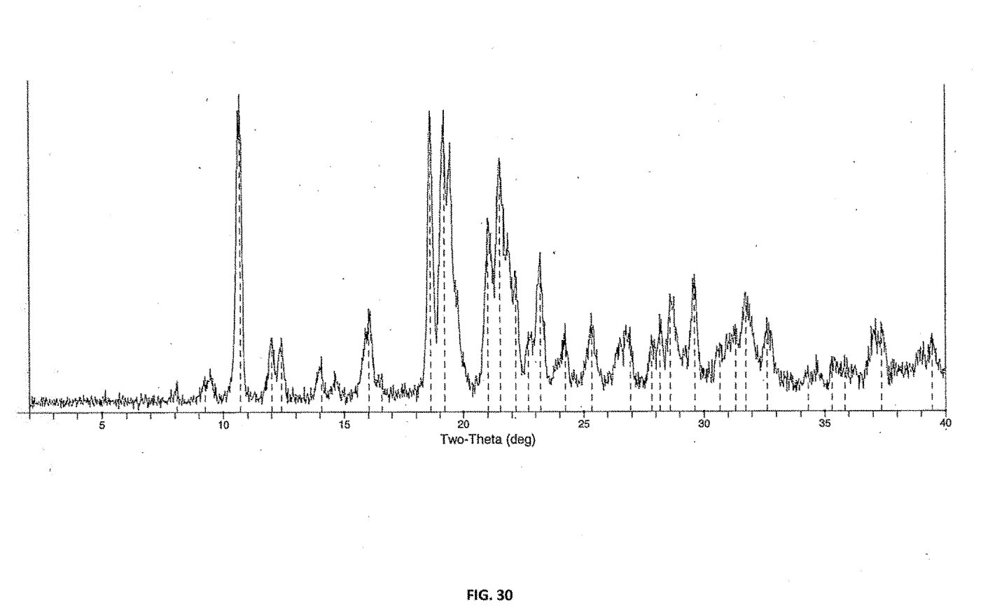

The XRPD of the bis-methanol solvate of the free base of (R)—N-(4-(3-aminopiperidin-1-yl)-5-bromo-1H-pyrrolo[2,3-b]pyridin-3-yl)cyclopropanecarboxamide, as calculated from the SCXRD data at 100 K, is shown in FIG. 30 and summarized in Table 30 below.

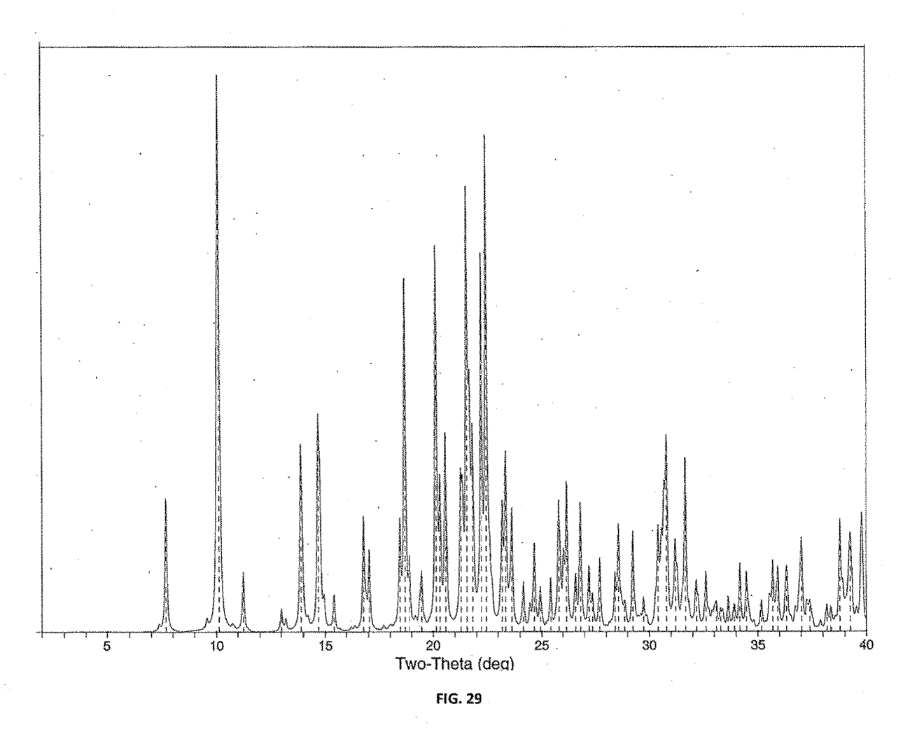

TABLE 30

| | |
|---|---|
| XRPD | FIG. 29: Peaks of ≥30% relative height at about 10.1, 13.9, |
| | 14.7, 18.7, 20.1, 20.6, 21.6, 21.8, 22.2, 22.4, 23.4, and |
| | 30.8 °2θ. |

Certain calculated XRPD diffraction peaks for the bis-methanol solvate of the free base of (R)—N-(4-(3-aminopiperidin-1-yl)-5-bromo-1H-pyrrolo[2,3-b]pyridin-3-yl)cyclopropanecarboxamide are shown in Table 31 below.

TABLE 31
SCAN: 2.0/40.0/0.02/0.1(sec), Cu, I(p) = 10000, 02/16/16 09:58a
PEAK: 15(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 7.683 | 11.4979 | 7 | 2372 | 23.8 | 17496 | 18.8 | 0.125 |
| 2 | 10.119 | 8.7342 | 31 | 9969 | 100.0 | 92690 | 99.5 | 0.158 |
| 3 | 11.259 | 7.8526 | 31 | 1036 | 10.4 | 6116 | 6.6 | 0.100 |
| 4 | 13.018 | 6.7953 | 39 | 372 | 3.7 | 2111 | 2.3 | 0.096 |
| 5 | 13.902 | 6.3648 | 65 | 3284 | 32.9 | 26858 | 28.8 | 0.139 |
| 6 | 14.682 | 6.0285 | 63 | 3837 | 38.5 | 40955 | 43.9 | 0.181 |
| 7 | 15.403 | 5.7478 | 26 | 636 | 6.4 | 5053 | 5.4 | 0.135 |
| 8 | 16.781 | 5.2788 | 26 | 2038 | 20.4 | 18785 | 20.2 | 0.157 |
| 9 | 17.041 | 5.1989 | 27 | 1438 | 14.4 | 15701 | 16.8 | 0.186 |
| 10 | 18.461 | 4.8023 | 44 | 1991 | 20.0 | 22827 | 24.5 | 0.195 |
| 11 | 18.697 | 4.7421 | 63 | 6269 | 62.9 | 45796 | 49.1 | 0.124 |
| 12 | 18.899 | 4.6918 | 72 | 1292 | 13.0 | 32234 | 34.6 | 0.424 |
| 13 | 19.460 | 4.5579 | 72 | 1012 | 10.1 | 9151 | 9.8 | 0.154 |
| 14 | 20.142 | 4.4050 | 197 | 6728 | 67.5 | 51107 | 54.8 | 0.129 |
| 15 | 20.321 | 4.3666 | 204 | 2615 | 26.2 | 31856 | 34.2 | 0.207 |
| 16 | 20.580 | 4.3123 | 214 | 3350 | 33.6 | 23467 | 25.2 | 0.119 |
| 17 | 21.281 | 4.1717 | 228 | 2702 | 27.1 | 46637 | 50.0 | 0.293 |
| 18 | 21.561 | 4.1183 | 228 | 7755 | 77.8 | 93202 | 100.0 | 0.204 |
| 19 | 21.819 | 4.0701 | 228 | 3497 | 35.1 | 73763 | 79.1 | 0.359 |
| 20 | 22.222 | 3.9972 | 228 | 6561 | 65.8 | 52318 | 56.1 | 0.136 |
| 21 | 22.460 | 3.9554 | 228 | 8692 | 87.2 | 77673 | 83.3 | 0.152 |
| 22 | 23.202 | 3.8306 | 143 | 2207 | 22.1 | 32991 | 35.4 | 0.254 |
| 23 | 23.359 | 3.8051 | 143 | 3085 | 30.9 | 30407 | 32.6 | 0.168 |
| 24 | 23.639 | 3.7606 | 143 | 2070 | 20.8 | 17990 | 19.3 | 0.148 |
| 25 | 24.159 | 3.6809 | 142 | 751 | 7.5 | 4274 | 4.6 | 0.097 |
| 26 | 24.663 | 3.6068 | 140 | 1438 | 14.4 | 10531 | 11.3 | 0.125 |
| 27 | 24.940 | 3.5674 | 139 | 665 | 6.7 | 4693 | 5.0 | 0.120 |
| 28 | 25.419 | 3.5012 | 138 | 823 | 8.3 | 4821 | 5.2 | 0.100 |
| 29 | 25.820 | 3.4477 | 138 | 2207 | 22.1 | 21569 | 23.1 | 0.166 |
| 30 | 26.182 | 3.4009 | 138 | 2546 | 25.5 | 25111 | 26.9 | 0.168 |
| 31 | 26.599 | 3.3485 | 138 | 902 | 9.0 | 9483 | 10.2 | 0.179 |
| 32 | 26.821 | 3.3213 | 138 | 2159 | 21.7 | 16220 | 17.4 | 0.128 |
| 33 | 27.222 | 3.2733 | 112 | 1058 | 10.6 | 8151 | 8.7 | 0.131 |
| 34 | 27.378 | 3.2550 | 112 | 584 | 5.9 | 7189 | 7.7 | 0.209 |
| 35 | 27.720 | 3.2156 | 112 | 1202 | 12.1 | 7229 | 7.8 | 0.102 |
| 36 | 28.421 | 3.1379 | 112 | 950 | 9.5 | 12173 | 13.1 | 0.218 |
| 37 | 28.581 | 3.1206 | 112 | 1805 | 18.1 | 26783 | 28.7 | 0.252 |
| 38 | 28.861 | 3.0910 | 112 | 450 | 4.5 | 10698 | 11.5 | 0.404 |
| 39 | 29.259 | 3.0498 | 112 | 1676 | 16.8 | 13308 | 14.3 | 0.135 |
| 40 | 29.741 | 3.0016 | 144 | 481 | 4.8 | 5532 | 5.9 | 0.196 |
| 41 | 30.419 | 2.9361 | 125 | 1779 | 17.8 | 34567 | 37.1 | 0.330 |
| 42 | 30.800 | 2.9007 | 125 | 3394 | 34.0 | 56300 | 60.4 | 0.282 |
| 43 | 31.201 | 2.8643 | 125 | 1522 | 15.3 | 18172 | 19.5 | 0.203 |
| 44 | 31.683 | 2.8218 | 125 | 2983 | 29.9 | 25452 | 27.3 | 0.145 |
| 45 | 32.178 | 2.7795 | 125 | 802 | 8.0 | 7982 | 8.6 | 0.169 |
| 46 | 32.622 | 2.7427 | 125 | 943 | 9.5 | 8138 | 8.7 | 0.147 |
| 47 | 33.097 | 2.7045 | 196 | 357 | 3.6 | 5065 | 5.4 | 0.241 |
| 48 | 33.301 | 2.6883 | 195 | 225 | 2.3 | 1819 | 2.0 | 0.137 |
| 49 | 33.643 | 2.6618 | 210 | 425 | 4.3 | 1684 | 1.8 | 0.067 |
| 50 | 33.920 | 2.6406. | 102 | 384 | 3.9 | 4371 | 4.7 | 0.194 |
| 51 | 34.180 | 2.6212 | 102 | 1116 | 11.2 | 7906 | 8.5 | 0.120 |
| 52 | 34.483 | 2.5988 | 102 | 976 | 9.8 | 9291 | 10.0 | 0.162 |
| 53 | 35.161 | 2.5503 | 102 | 457 | 4.6 | 2580 | 2.8 | 0.096 |
| 54 | 35.700 | 2.5130 | 102 | 1175 | 11.8 | 17948 | 19.3 | 0.260 |
| 55 | 35.938 | 2.4969 | 102 | 1071 | 10.7 | 11373 | 12.2 | 0.180 |
| 56 | 36.321 | 2.4714 | 102 | 1077 | 10.8 | 10553 | 11.3 | 0.167 |
| 57 | 37.019 | 2.4264 | 88 | 1583 | 15.9 | 19451 | 20.9 | 0.209 |
| 58 | 37.401 | 2.4025 | 88 | 484 | 4.9 | 8511 | 9.1 | 0.299 |
| 59 | 38.200 | 2.3541 | 88 | 393 | 3.9 | 3658 | 3.9 | 0.158 |
| 60 | 38.381 | 2.3434 | 88 | 352 | 3.5 | 3050 | 3.3 | 0.147 |
| 61 | 38.783 | 2.3200 | 93 | 1910 | 19.2 | 22138 | 23.8 | 0.197 |
| 62 | 39.259 | 2.2930 | 93 | 1672 | 16.8 | 24368 | 26.1 | 0.248 |

NOTE:
Intensity = Counts, 2T(0) = 0.0(deg), Wavelength to Compute d-Spacing = 1.54059 Å (Cu/K-alpha1)

### Example 17

(R)—N-(4-(3-aminopiperidin-1-yl)-5-bromo-1H-pyrrolo[2,3-b]pyridin-3-yl)cyclopropanecarboxamide methyl tert-butyl ether solvate: (R)—N-(4-(3-aminopiperidin-1-yl)-5-bromo-1H-pyrrolo[2,3-b]pyridin-3-yl)cyclopropanecarboxamide was dissolve at 200 mg/mL in pyridine at 65° C. The solution was diluted to 10 mg/mL with methyl tert-butyl ether and cooled from 65° C. to 10° C. over 14.5 h. The mixture was evaporated until dry to yield (R)—N-(4-(3-aminopiperidin-1-yl)-5-bromo-1H-pyrrolo[2,3-b]pyridin-3-yl)cyclopropanecarboxamide methyl tert-butyl ether solvate. The physical properties of the methyl tert-butyl ether solvate of the free base of (R)—N-(4-(3-aminopiperidin-1-yl)-5-bromo-1H-pyrrolo[2,3-b]pyridin-3-yl)cyclopropanecarboxamide are summarized in Table 32 below.

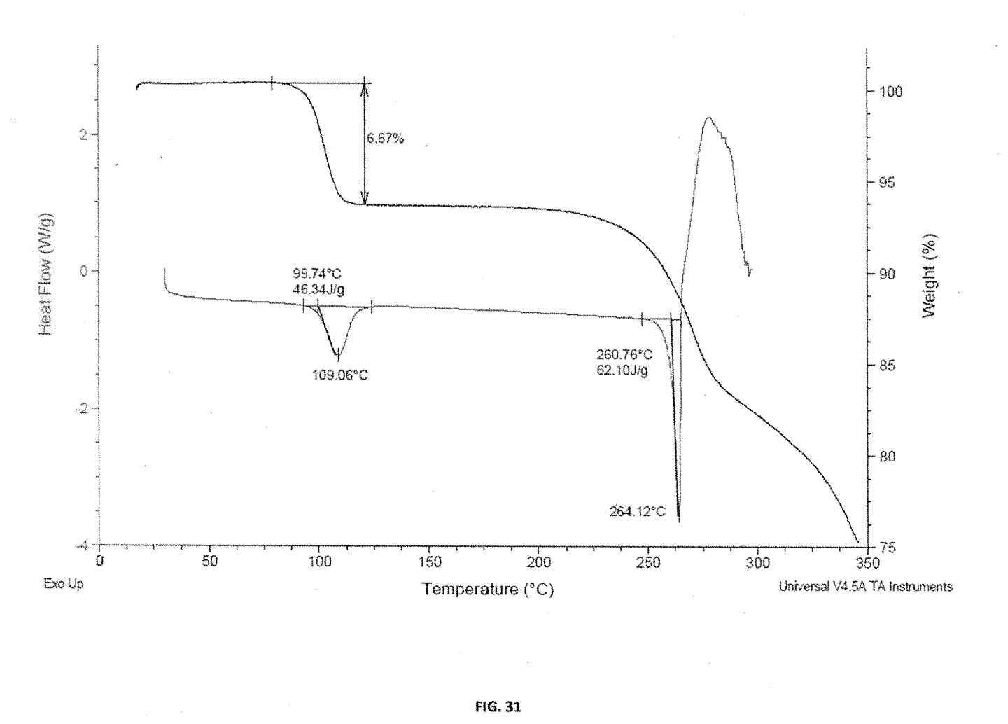

TABLE 32

| | |
|---|---|
| XRPD | FIG. 30: Peaks of ≥20% relative height at about |
| | 10.7, 16.0, 18.6, 19.2, 21.0, 21.5, 22.2, 23.2, |
| | 25.3, 28.1, 28.6, 29.6, and 31.8 °2θ. |
| DSC | FIG. 31: Endotherm with extrapolated onset temperature |
| | about 100° C. and enthalpy of fusion about 46 J/g. |
| | Endotherm with extrapolated onset temperature about |
| | 261° C. and enthalpy of fusion about 62 J/g. |
| TGA | FIG. 31: About 6.7% weight loss up to about 120° C. |

Certain XRPD diffraction peaks for the methyl tert-butyl ether solvate of the free base of (R)—N-(4-(3-aminopiperidin-1-yl)-5-bromo-1H-pyrrolo[2,3-b]pyridin-3-yl)cyclopropanecarboxamide are shown in Table 33 below.

TABLE 33
SCAN: 2.0/40.0/0.02/0.6(sec), Cu(30 kV, 15 mA),
I(p) = 391.0, 09/21/11 10:42a
PEAK: 29(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 8.092 | 10.9179 | 14 | 24 | 6.4 | 149 | 1.2 | 0.105 |
| 2 | 9.257 | 9.5457 | 15 | 24 | 6.4 | 590 | 4.7 | 0.420 |
| 3 | 10.700 | 8.2614 | 17 | 374 | 100.0 | 5094 | 41.0 | 0.231 |
| 4 | 12.019 | 7.3578 | 18 | 74 | 19.7 | 1335 | 10.7 | 0.307 |
| 5 | 12.421 | 7.1204 | 18 | 73 | 19.4 | 897 | 7.2 | 0.210 |
| 6 | 14.077 | 6.2863 | 17 | 51 | 13.6 | 703 | 5.7 | 0.235 |
| 7 | 16.022 | 5.5272 | 19 | 108 | 28.7 | 2453 | 19.7 | 0.388 |
| 8 | 16.589 | 5.3395 | 19 | 28 | 7.4 | 163 | 1.3 | 0.101 |
| 9 | 18.620 | 4.7614 | 24 | 346 | 92.5 | 4890 | 39.3 | 0.240 |
| 10 | 19.219 | 4.6145 | 38 | 333 | 89.0 | 11412 | 91.8 | 0.583 |
| 11 | 21.023 | 4.2224 | 31 | 208 | 55.6 | 6035 | 48.6 | 0.493 |
| 12 | 21.520 | 4.1260 | 37 | 275 | 73.6 | 12429 | 100.0 | 0.767 |
| 13 | 22.179 | 4.0048 | 37 | 136 | 36.5 | 1548 | 12.5 | 0.193 |
| 14 | 22.702 | 3.9137 | 38 | 60 | 16.0 | 1596 | 12.8 | 0.452 |
| 15 | 23.218 | 3.8278 | 38 | 158 | 42.2 | 3401 | 27.4 | 0.366 |
| 16 | 24.238 | 3.6690 | 37 | 71 | 19.1 | 1053 | 8.5 | 0.251 |
| 17 | 25.338 | 3.5122 | 36 | 85 | 22.7 | 1394 | 11.2 | 0.279 |
| 18 | 26.938 | 3.3071 | 39 | 65 | 17.4 | 1558 | 12.5 | 0.407 |
| 19 | 27.840 | 3.2020 | 36 | 58 | 15.4 | 987 | 7.9 | 0.292 |
| 20 | 28.181 | 3.1640 | 36 | 83 | 22.1 | 1270 | 10.2 | 0.262 |
| 21 | 28.622 | 3.1163 | 38 | 106 | 28.4 | 3296 | 26.5 | 0.526 |
| 22 | 29.620 | 3.0135 | 44 | 124 | 33.2 | 1757 | 14.1 | 0.240 |
| 23 | 30.683 | 2.9115 | 45 | 39 | 10.4 | 470 | 3.8 | 0.205 |
| 24 | 31.320 | 2.8537 | 51 | 56 | 14.8 | 2833 | 22.8 | 0.868 |
| 25 | 31.758 | 2.8153 | 63 | 83 | 22.1 | 2226 | 17.9 | 0.459 |
| 26 | 32.621 | 2.7428 | 55 | 60 | 16.2 | 795 | 6.4 | 0.224 |
| 27 | 34.298 | 2.6124 | 31 | 25 | 6.6 | 347 | 2.8 | 0.240 |
| 28 | 35.304 | 2.5402 | 35 | 32 | 8.6 | 566 | 4.6 | 0.299 |
| 29 | 35.839 | 2.5035 | 37 | 31 | 8.4 | 897 | 7.2 | 0.485 |
| 30 | 37.345 | 2.4060 | 42 | 67 | 18.0 | 1778 | 14.3 | 0.449 |
| 31 | 39.459 | 2.2818 | 49 | 46 | 12.2 | 939 | 7.6 | 0.350 |

NOTE:
Intensity = Counts, 2T(0) = 0.0(deg), Wavelength to Compute d-Spacing = 1.54059 Å (Cu/K-alpha1)

### Example 18 (R)—N-(4-(3-aminopiperidin-1-yl)-5-bromo-1H-pyrrolo[2,3-b]pyridin-3-yl)cyclopropanecarboxamide toluene solvate

Method A: (R)—N-(4-(3-aminopiperidin-1-yl)-5-bromo-1H-pyrrolo[2,3-b]pyridin-3-yl)cyclopropanecarboxamide (26.4 mg) was heated in toluene (5 mL) at 50° C. in a shaker block for 22 days. The sample was cooled to ambient and the solid removed and analyzed by XRPD while still damp with solvent.

Method B: (R)—N-(4-(3-aminopiperidin-1-yl)-5-bromo-1H-pyrrolo[2,3-b]pyridin-3-yl)cyclopropanecarboxamide (28.8 mg) was slurried in toluene (5 mL) at 25° C. in a shaker block for 24 days. The solid was removed and analyzed by XRPD while still damp with solvent.

Method C: (R)—N-(4-(3-aminopiperidin-1-yl)-5-bromo-1H-pyrrolo[2,3-b]pyridin-3-yl)cyclopropanecarboxamide was dissolved at 10 mg/mL in toluene at 65° C. The solution was cooled from 65° C. to 10° C. over 14.5 h, then held at 5° C. until crystals formed. Solids were isolated via filtration to yield (R)—N-(4-(3-aminopiperidin-1-yl)-5-bromo-1H-pyrrolo[2,3-b]pyridin-3-yl)cyclopropanecarboxamide toluene solvate.

The physical properties of the toluene solvate of the free base of (R)—N-(4-(3-aminopiperidin-1-yl)-5-bromo-1H-pyrrolo[2,3-b]pyridin-3-yl)cyclopropanecarboxamide are summarized in Table 34 below.

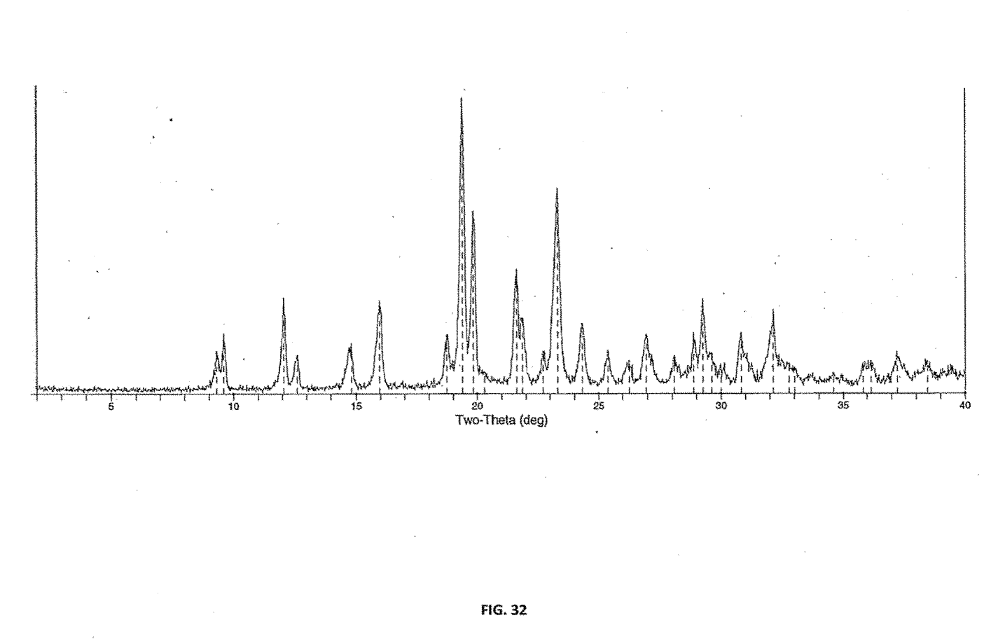

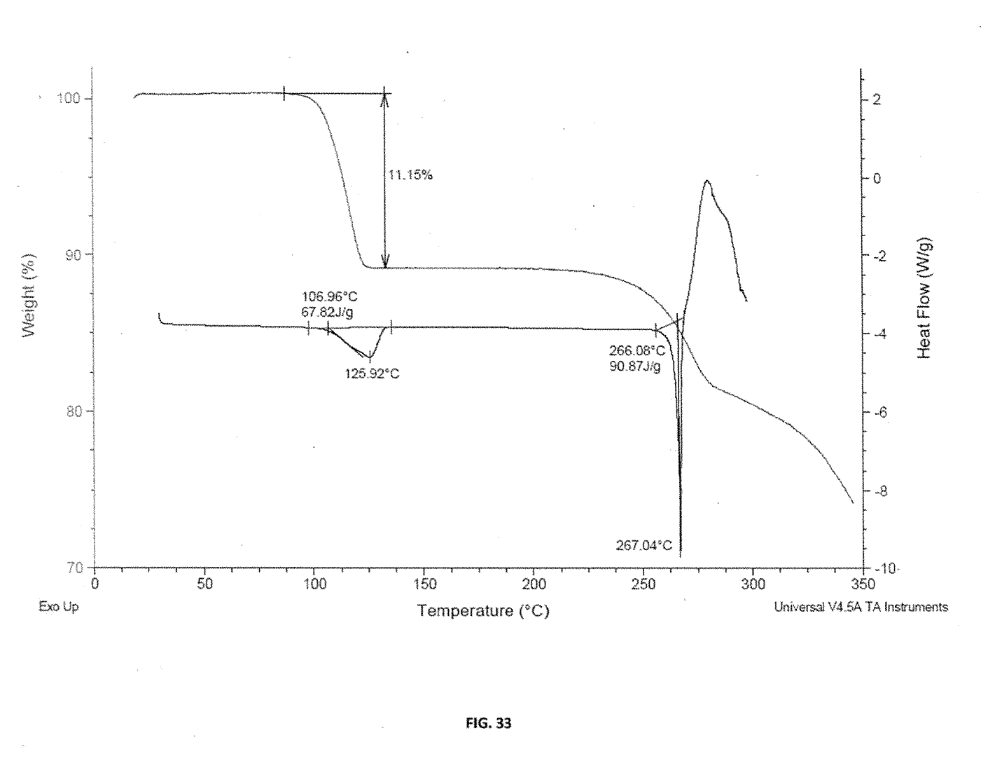

TABLE 34

| | |
|---|---|
| XRPD | FIG. 32: Peaks of ≥20% relative height at about |
| | 12.1, 16.0, 19.4, 19.8, 21.6, 21.9, 23.3, 24.3, 29.3, |
| | and 32.1 °2θ. |
| DSC | FIG. 33: Endotherm with extrapolated onset temperature |
| | about 107° C. and enthalpy of fusion about 67 J/g. |
| | Endotherm with extrapolated onset temperature about |
| | 266° C. and enthalpy of fusion about 91 J/g. |
| TGA | FIG. 33: About 11.2% weight loss up to about 140° C. |

Certain XRPD diffraction peaks for the toluene solvate of the free base of (R)—N-(4-(3-aminopiperidin-1-yl)-5-bromo-1H-pyrrolo[2,3-b]pyridin-3-yl)cyclopropanecarboxamide are shown in Table 35 below.

TABLE 35
SCAN: 2.0/40.0/0.02/0.6(sec), Cu(30 kV,15 mA),
I(p) = 778.0, 11/12/12 02:43p
PEAK: 21 (pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 9.302 | 9.5000 | 13 | 99 | 13.0 | 1451 | 13.0 | 0.250 |
| 2 | 9.603 | 9.2023 | 13 | 144 | 18.9 | 1514 | 13.6 | 0.179 |
| 3 | 12.062 | 7.3312 | 12 | 239 | 31.4 | 2962 | 26.5 | 0.211 |
| 4 | 12.619 | 7.0090 | 13 | 88 | 11.6 | 1186 | 10.6 | 0.229 |
| 5 | 14.818 | 5.9737 | 18 | 114 | 15.0 | 1483 | 13.3 | 0.222 |
| 6 | 15.961 | 5.5483 | 19 | 225 | 29.6 | 3111 | 27.9 | 0.235 |
| 7 | 18.742 | 4.7306 | 19 | 137 | 18.1 | 2109 | 18.9 | 0.261 |
| 8 | 19.381 | 4.5763 | 19 | 759 | 100.0 | 11168 | 100.0 | 0.250 |
| 9 | 19.841 | 4.4711 | 19 | 460 | 60.6 | 7237 | 64.8 | 0.267 |
| 10 | 20.299 | 4.3713 | 19 | 38 | 5.1 | 908 | 8.1 | 0.402 |
| 11 | 21.619 | 4.1072 | 33 | 293 | 38.6 | 5096 | 45.6 | 0.296 |
| 12 | 21.860 | 4.0626 | 32 | 166 | 21.8 | 3411 | 30.5 | 0.350 |
| 13 | 22.719 | 3.9109 | 34 | 78 | 10.2 | 1338 | 12.0 | 0.293 |
| 14 | 23.318 | 3.8117 | 34 | 506 | 66.7 | 7880 | 70.6 | 0.265 |
| 15 | 24.320 | 3.6569 | 29 | 156 | 20.5 | 2135 | 19.1 | 0.233 |
| 16 | 25.399 | 3.5039 | 26 | 89 | 11.7 | 1051 | 9.4 | 0.202 |
| 17 | 26.259 | 3.3911 | 31 | 55 | 7.2 | 653 | 5.8 | 0.202 |
| 18 | 26.940 | 3.3069 | 33 | 122 | 16.0 | 2093 | 18.7 | 0.293 |
| 19 | 28.083 | 3.1748 | 30 | 69 | 9.1 | 1129 | 10.1 | 0.277 |
| 20 | 28.883 | 3.0887 | 35 | 124 | 16.4 | 2618 | 23.4 | 0.358 |
| 21 | 29.259 | 3.0499 | 52 | 196 | 25.8 | 3557 | 31.8 | 0.308 |
| 22 | 29.616 | 3.0139 | 33 | 74 | 9.7 | 3454 | 30.9 | 0.795 |
| 23 | 29.993 | 2.9769 | 33 | 40 | 5.2 | 535 | 4.8 | 0.228 |
| 24 | 30.819 | 2.8990 | 36 | 124 | 16.4 | 2055 | 18.4 | 0.281 |
| 25 | 32.139 | 2.7828 | 43 | 176 | 23.2 | 3535 | 31.6 | 0.341 |
| 26 | 32.797 | 2.7285 | 31 | 48 | 6.4 | 1072 | 9.6 | 0.376 |
| 27 | 33.021 | 2.7105 | 31 | 38 | 5.1 | 326 | 2.9 | 0.144 |
| 28 | 34.603 | 2.5901 | 29 | 25 | 3.2 | 434 | 3.9 | 0.300 |
| 29 | 35.838 | 2.5036 | 29 | 51 | 6.7 | 1247 | 11.2 | 0.417 |
| 30 | 36.176 | 2.4810 | 31 | 54 | 7.1 | 1247 | 11.2 | 0.391 |
| 31 | 37.222 | 2.4136 | 36 | 74 | 9.8 | 1282 | 11.5 | 0.294 |
| 32 | 38.458 | 2.3389 | 42 | 44 | 5.8 | 819 | 7.3 | 0.314 |

NOTE:
Intensity = Counts, 2T(0) = 0.0(deg), Wavelength to Compute d-Spacing = 1.54059 Å (Cu/K-alpha1)

### Example 19

(R)—N-(4-(3-aminopiperidin-1-yl)-5-bromo-1H-pyrrolo[2,3-b]pyridin-3-yl)cyclopropanecarboxamide butyronitrile solvate: (R)—N-(4-(3-aminopiperidin-1-yl)-5-bromo-1H-pyrrolo[2,3-b]pyridin-3-yl)cyclopropanecarboxamide was dissolved at 500 mg/mL in 2,2,2-trifluoroethanol at 65° C. The solution was diluted to 25 mg/mL with butyronitrile and cooled from 65° C. to 10° C. over 14.5 h. Solids were isolated via filtration to yield (R)—N-(4-(3-aminopiperidin-1-yl)-5-bromo-1H-pyrrolo[2,3-b]pyridin-3-yl)cyclopropanecarboxamide butyronitrile solvate. The physical properties of the butyronitrile solvate of the free base of (R)—N-(4-(3-aminopiperidin-1-yl)-5-bromo-1H-pyrrolo[2,3-b]pyridin-3-yl)cyclopropanecarboxamide are summarized in Table 36 below.

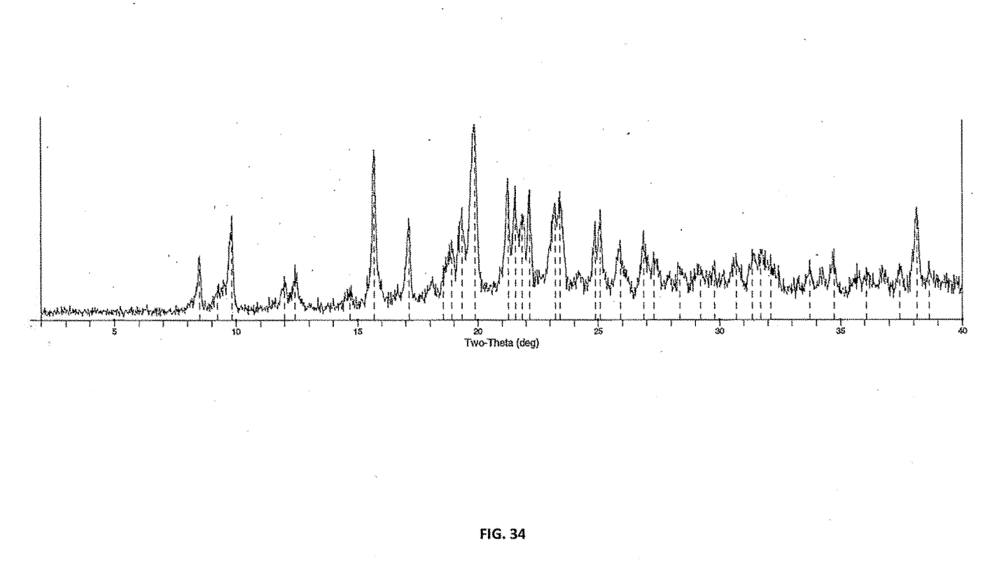

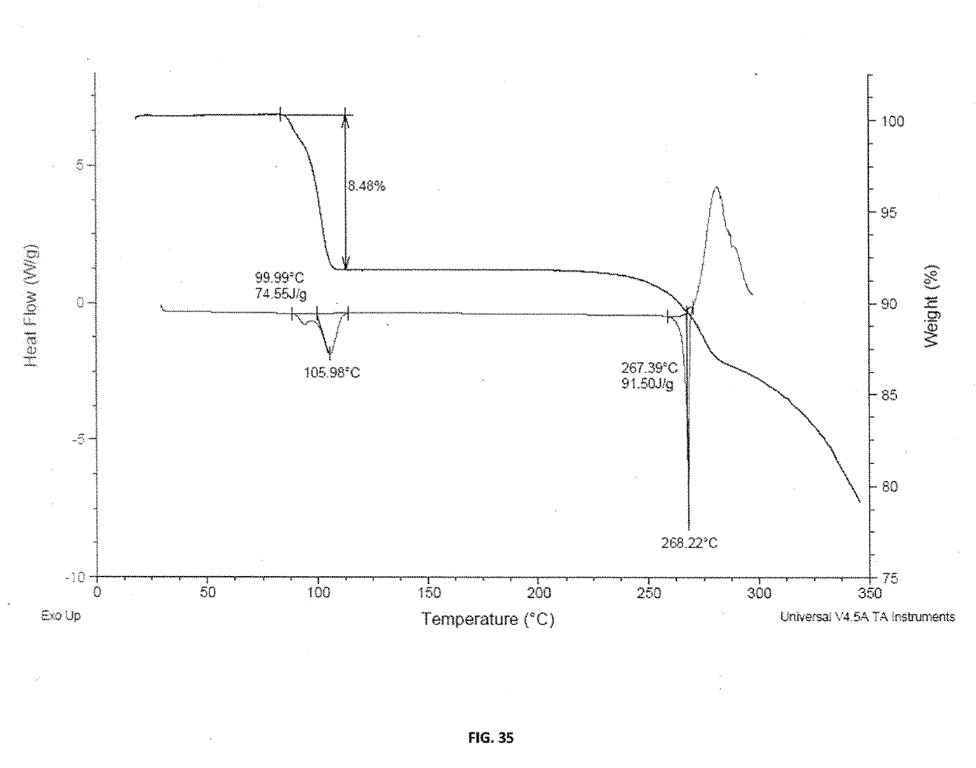

TABLE 36

| | |
|---|---|
| XRPD | FIG. 34: Peaks of ≥40% relative height at about |
| | 9.8, 15.7, 17.1, 19.4, 19.9, 21.3, 21.6, 22.2, 23.2, |
| | 23.4, 25.1, and 47.9 °2θ. |
| DSC | FIG. 35: Endotherm with extrapolated onset temperature |
| | about 100° C. and enthalpy of fusion about 75 J/g. |
| | Endotherm with extrapolated onset temperature about |
| | 267° C. and enthalpy of fusion about 92 J/g. |
| TGA | FIG. 35: About 8.5% weight loss up to about 120° C. |

Certain XRPD diffraction peaks for the butyronitrile solvate of the free base of (R)—N-(4-(3-aminopiperidin-1-yl)-5-bromo-1H-pyrrolo[2,3-b]pyridin-3-yl)cyclopropanecarboxamide are shown in Table 37 below.

TABLE 37
SCAN: 2.0/40.0/0.02/0.6(sec), Cu(30 kV, 15 mA),
I(p) = 336.0, 09/20/11 05:02p
PEAK: 21(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 8.496 | 10.3991 | 21 | 88 | 31.2 | 867 | 16.5 | 0.167 |
| 2 | 9.242 | 9.5611 | 23 | 29 | 10.2 | 949 | 18.1 | 0.559 |
| 3 | 9.840 | 8.9816 | 19 | 160 | 56.6 | 2402 | 45.7 | 0.256 |
| 4 | 12.001 | 7.3687 | 23 | 51 | 18.2 | 844 | 16.1 | 0.280 |
| 5 | 12.440 | 7.1095 | 23 | 71 | 25.3 | 838 | 16.0 | 0.200 |
| 6 | 14.677 | 6.0306 | 24 | 31 | 11.0 | 361 | 6.9 | 0.198 |
| 7 | 15.681 | 5.6466 | 32 | 260 | 92.0 | 2847 | 54.2 | 0.186 |
| 8 | 17.141 | 5.1688 | 37 | 137 | 48.5 | 1362 | 25.9 | 0.169 |
| 9 | 18.550 | 4.7793 | 37 | 55 | 19.4 | 671 | 12.8 | 0.208 |
| 10 | 18.901 | 4.6914 | 46 | 89 | 31.4 | 1540 | 29.3 | 0.295 |
| 11 | 19.358 | 4.5817 | 58 | 134 | 47.4 | 2791 | 53.1 | 0.355 |
| 12 | 19.879 | 4.4626 | 54 | 282 | 100.0 | 5253 | 100.0 | 0.316 |
| 13 | 21.257 | 4.1764 | 54 | 189 | 67.1 | 3225 | 61.4 | 0.290 |
| 14 | 21.558 | 4.1188 | 61 | 169 | 59.9 | 5075 | 96.6 | 0.510 |
| 15 | 21.840 | 4.0661 | 69 | 112 | 39.6 | 2629 | 50.1 | 0.400 |
| 16 | 22.176 | 4.0054 | 67 | 156 | 55.3 | 1253 | 23.8 | 0.136 |
| 17 | 23.218 | 3.8279 | 70 | 130 | 46.2 | 3182 | 60.6 | 0.415 |
| 18 | 23.420 | 3.7954 | 66 | 155 | 54.9 | 3630 | 69.1 | 0.398 |
| 19 | 24.877 | 3.5762 | 59 | 109 | 38.6 | 1542 | 29.4 | 0.241 |
| 20 | 25.101 | 3.5449 | 58 | 131 | 46.3 | 1584 | 30.1 | 0.206 |
| 21 | 25.916 | 3.4352 | 53 | 82 | 29.1 | 1257 | 23.9 | 0.260 |
| 22 | 26.878 | 3.3144 | 55 | 97 | 34.5 | 1739 | 33.1 | 0.304 |
| 23 | 27.299 | 3.2642 | 54 | 61 | 21.6 | 1367 | 26.0 | 0.380 |
| 24 | 28.360 | 3.1445 | 58 | 33 | 11.8 | 414 | 7.9 | 0.211 |
| 25 | 29.220 | 3.0538 | 61 | 29 | 10.2 | 579 | 11.0 | 0.343 |
| 26 | 29.796 | 2.9961 | 69 | 31 | 11.0 | 321 | 6.1 | 0.176 |
| 27 | 30.701 | 2.9098 | 67 | 46 | 16.3 | 380 | 7.2 | 0.140 |
| 28 | 31.364 | 2.8498 | 67 | 53 | 18.8 | 1415 | 26.9 | 0.452 |
| 29 | 31.705 | 2.8199 | 60 | 60 | 21.2 | 2242 | 42.7 | 0.638 |
| 30 | 32.118 | 2.7846 | 55 | 55 | 19.5 | 2379 | 45.3 | 0.735 |
| 31 | 33.740 | 2.6544 | 55 | 46 | 16.2 | 549 | 10.4 | 0.204 |
| 32 | 34.736 | 2.5805 | 55 | 65 | 23.2 | 532 | 10.1 | 0.138 |
| 33 | 36.083 | 2.4872 | 48 | 40 | 14.2 | 1559 | 29.7 | 0.661 |
| 34 | 37.424 | 2.4011 | 56 | 39 | 14.0 | 268 | 5.1 | 0.115 |
| 35 | 38.140 | 2.3577 | 57 | 135 | 47.9 | 1884 | 35.9 | 0.237 |
| 36 | 38.640 | 2.3283 | 60 | 39 | 14.0 | 617 | 11.7 | 0.266 |

NOTE:
Intensity = Counts, 2T(0) = 0.0(deg), Wavelength to Compute d-Spacing = 1.54050 Å (Cu/K-alpha1)

### Example 20 (R)—N-(4-(3-aminopiperidin-1-yl)-5-bromo-1H-pyrrolo[2,3-b]pyridin-3-yl)cyclopropanecarboxamide di-methanesulfonic acid salt hydrate

Method A: (R)—N-(4-(3-aminopiperidin-1-yl)-5-bromo-1H-pyrrolo[2,3-b]pyridin-3-yl)cyclopropanecarboxamide (40 mg, 0.11 mmol) was suspended in about 1 mL of acetone. Methanesulfonic acid (0.22 mmol, 20.3 mg) was added and the resulting suspension stirred for one day. The solids were isolated by centrifugation.

Method B: (R)—N-(4-(3-aminopiperidin-1-yl)-5-bromo-1H-pyrrolo[2,3-b]pyridin-3-yl)cyclopropanecarboxamide (40 mg, 0.11 mmol) was dissolved in about 1 mL of tetrahydrofuran. Methanesulfonic acid (0.22 mmol, 20.3 mg) was added and the resulting suspension stirred for one day. The solids were isolated by centrifugation.

The physical properties of the hydrate of the di-mesylate salt of (R)—N-(4-(3-aminopiperidin-1-yl)-5-bromo-1H-pyrrolo[2,3-b]pyridin-3-yl)cyclopropanecarboxamide are summarized in Table 38 below.

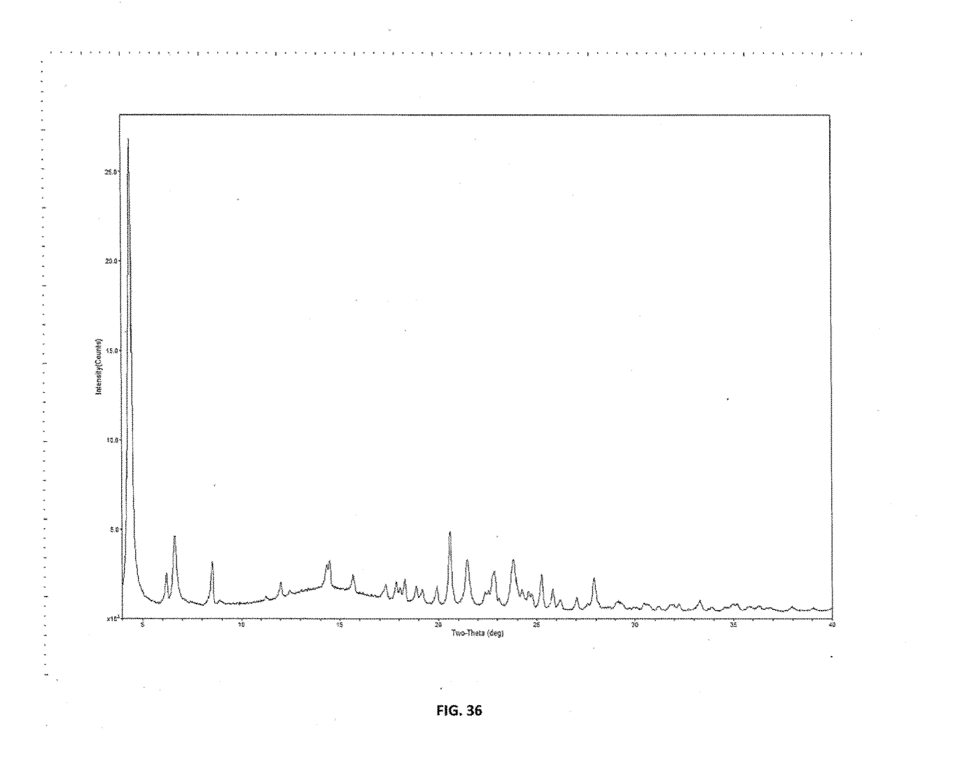

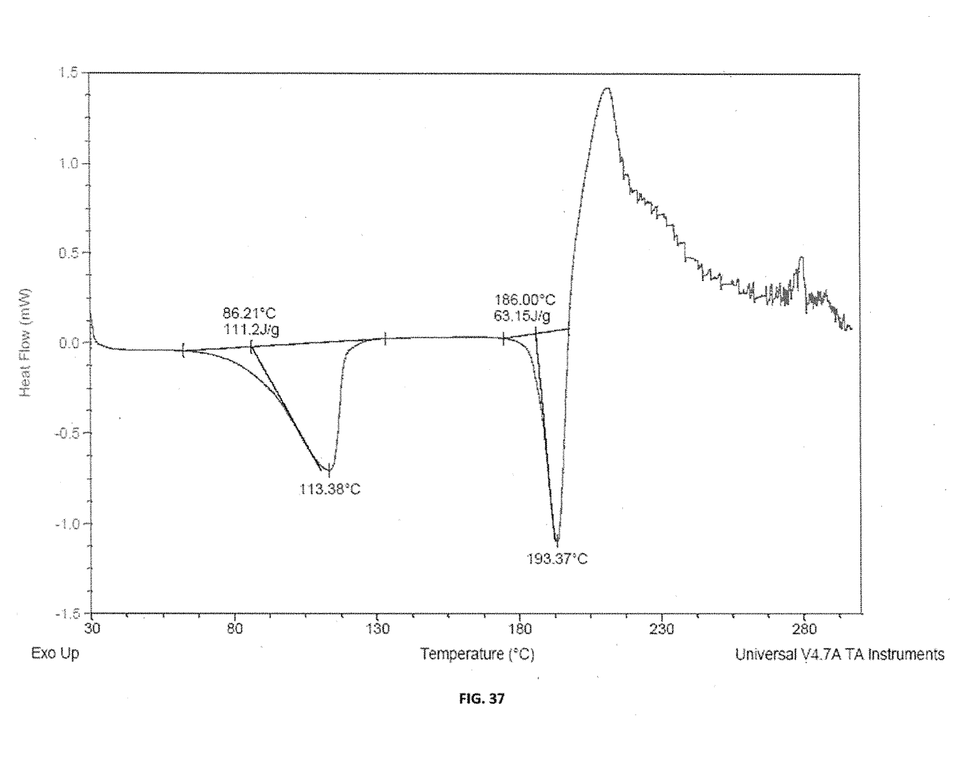

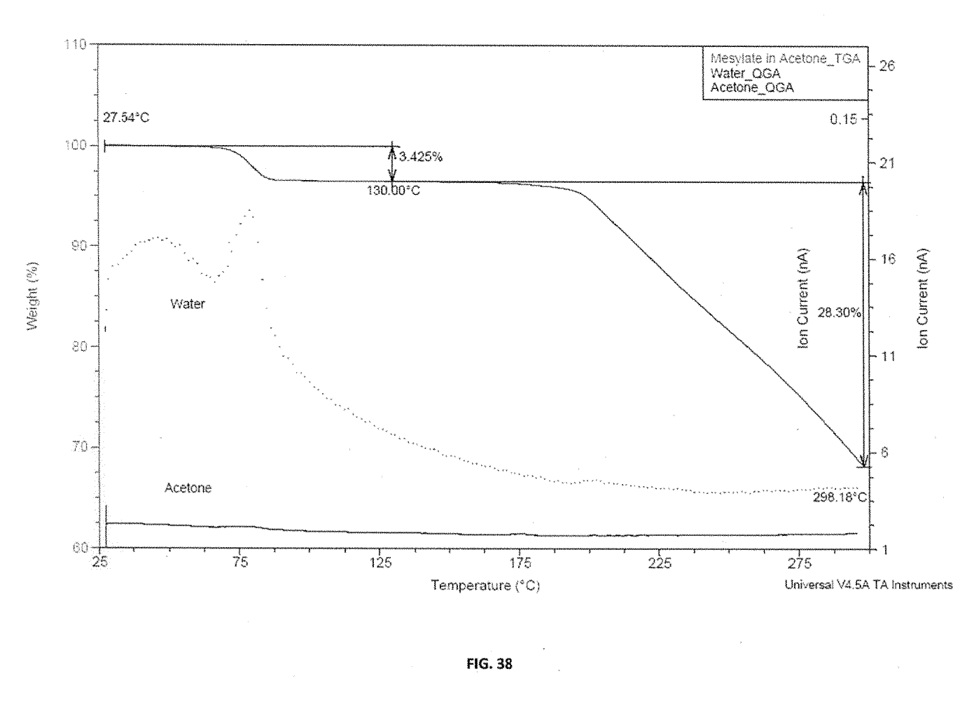

TABLE 38

| | |
|---|---|
| XRPD | FIG. 36: Peaks of ≥10% relative height at about 4.4, |
| | 6.2, 6.7, 8.5, 14.5, 20.6, 21.4, 22.9, 23.9, 25.3, and |
| | 27.9 °2θ. |
| DSC | FIG. 37: Endotherm with extrapolated onset temperature |
| | about 86° C. and enthalpy of fusion about 111 J/g. |
| | Endotherm with extrapolated onset temperature about |
| | 186° C. and enthalpy of fusion about 63 J/g. |
| TGA | FIG. 38: About 3.4% weight loss up to about130° C. |

Certain XRPD diffraction peaks for the hydrate of the di-mesylate salt of (R)—N-(4-(3-aminopiperidin-1-yl)-5-bromo-1H-pyrrolo[2,3-b]pyridin-3-yl)cyclopropanecarboxamide are shown in Table 39 below.

TABLE 39
SCAN: 4.0/40.0055/0.01975/21.6(sec), Cu(40 kV, 40 mA),
I(p) = 26838, 09/08/13 05:40p
PEAK: 19(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 4.435 | 19.9098 | 1024 | 25814 | 100.0 | 340263 | 100.0 | 0.221 |
| 2 | 6.232 | 14.1708 | 921 | 1630 | 6.3 | 14494 | 4.3 | 0.149 |
| 3 | 6.666 | 13.2489 | 883 | 3759 | 14.6 | 58061 | 17.1 | 0.259 |
| 4 | 8.541 | 10.3441 | 810 | 2387 | 9.2 | 20384 | 6.0 | 0.143 |
| 5 | 8.916 | 9.9106 | 829 | 236 | 0.9 | 2550 | 0.7 | 0.182 |
| 6 | 12.021 | 7.3563 | 1226 | 819 | 3.2 | 6353 | 1.9 | 0.130 |
| 7 | 12.492 | 7.0802 | 1358 | 239 | 0.9 | 1959 | 0.6 | 0.137 |
| 8 | 14.488 | 6.1089 | 1649 | 1564 | 6.1 | 27687 | 8.1 | 0.297 |
| 9 | 15.696 | 5.6414 | 1540 | 913 | 3.5 | 8066 | 2.4 | 0.148 |
| 10 | 17.349 | 5.1073 | 1205 | 705 | 2.7 | 7104 | 2.1 | 0.169 |
| 11 | 17.868 | 4.9602 | 1123 | 939 | 3.6 | 11325 | 3.3 | 0.202 |
| 12 | 18.317 | 4.8395 | 1037 | 1175 | 4.6 | 9978 | 2.9 | 0.143 |
| 13 | 18.897 | 4.6924 | 966 | 859 | 3.3 | 10263 | 3.0 | 0.200 |
| 14 | 19.225 | 4.6129 | 944 | 729 | 2.8 | 7890 | 2.3 | 0.182 |
| 15 | 19.958 | 4.4452 | 877 | 961 | 3.7 | 7303 | 2.1 | 0.128 |
| 16 | 20.628 | 4.3023 | 885 | 3999 | 15.5 | 39012 | 11.5 | 0.164 |
| 17 | 21.462 | 4.1370 | 857 | 2418 | 9.4 | 35879 | 10.5 | 0.249 |
| 18 | 22.409 | 3.9642 | 796 | 731 | 2.8 | 26557 | 7.8 | 0.610 |
| 19 | 22.899 | 3.8804 | 793 | 1862 | 7.2 | 38499 | 11.3 | 0.347 |
| 20 | 23.868 | 3.7251 | 793 | 2524 | 9.8 | 45137 | 13.3 | 0.300 |
| 21 | 24.266 | 3.6650 | 748 | 919 | 3.6 | 20859 | 6.1 | 0.381 |
| 22 | 24.637 | 3.6106 | 658 | 833 | 3.2 | 13491 | 4.0 | 0.272 |
| 23 | 25.270 | 3.5215 | 681 | 1805 | 7.0 | 15904 | 4.7 | 0.148 |
| 24 | 25.846 | 3.4443 | 611 | 1075 | 4.2 | 9921 | 2.9 | 0.155 |
| 25 | 26.220 | 3.3960 | 561 | 518 | 2.0 | 5560 | 1.6 | 0.180 |
| 26 | 27.088 | 3.2892 | 541 | 668 | 2.6 | 5678 | 1.7 | 0.143 |
| 27 | 27.621 | 3.2269 | 589 | 273 | 1.1 | 4261 | 1.3 | 0.262 |
| 28 | 27.939 | 3.1909 | 575 | 1720 | 6.7 | 23567 | 6.9 | 0.230 |
| 29 | 29.182 | 3.0578 | 603 | 438 | 1.7 | 7751 | 2.3 | 0.297 |
| 30 | 29.975 | 2.9786 | 564 | 122 | 0.5 | 1078 | 0.3 | 0.148 |
| 31 | 30.504 | 2.9282 | 532 | 361 | 1.4 | 6266 | 1.8 | 0.291 |
| 32 | 31.197 | 2.8646 | 514 | 197 | 0.8 | 1649 | 0.5 | 0.140 |
| 33 | 31.908 | 2.8024 | 490 | 321 | 1.2 | 7424 | 2.2 | 0.388 |
| 34 | 32.279 | 2.7710 | 480 | 368 | 1.4 | 4902 | 1.4 | 0.224 |
| 35 | 33.329 | 2.6861 | 457 | 603 | 2.3 | 9548 | 2.8 | 0.266 |
| 36 | 33.958 | 2.6378 | 457 | 193 | 0.7 | 2424 | 0.7 | 0.211 |
| 37 | 34.614 | 2.5893 | 457 | 209 | 0.8 | 5124 | 1.5 | 0.412 |
| 38 | 35.225 | 2.5458 | 486 | 368 | 1.4 | 10668 | 3.1 | 0.487 |
| 39 | 35.839 | 2.5036 | 511 | 188 | 0.7 | 3531 | 1.0 | 0.315 |
| 40 | 36.312 | 2.4720 | 469 | 276 | 1.1 | 8435 | 2.5 | 0.512 |
| 41 | 36.866 | 2.4362 | 497 | 159 | 0.6 | 2224 | 0.7 | 0.235 |
| 42 | 38.009 | 2.3655 | 458 | 257 | 1.0 | 2647 | 0.8 | 0.173 |
| 43 | 39.099 | 2.3020 | 453 | 181 | 0.7 | 1846 | 0.5 | 0.171 |

NOTE:
Intensity = Counts, 2T(0) = 0.0(deg), Wavelength to Compute d-Spacing = 1.54059 Å (Cu/K-alpha1)

### Example 21 (R)—N-(4-(3-aminopiperidin-1-yl)-5-bromo-1H-pyrrolo[2,3-b]pyridin-3-yl)cyclopropanecarboxamide di-ethanesulfonic acid salt hydrate

Method A: (R)—N-(4-(3-aminopiperidin-1-yl)-5-bromo-1H-pyrrolo[2,3-b]pyridin-3-yl)cyclopropanecarboxamide (40 mg, 0.11 mmol) was dissolved in about 1 mL of tetrahydrofuran. Ethanesulfonic acid (0.22 mmol, 24.5 mg) was added and the resulting suspension stirred for one day. The solids were isolated by centrifugation.

Method B: (R)—N-(4-(3-aminopiperidin-1-yl)-5-bromo-1H-pyrrolo[2,3-b]pyridin-3-yl)cyclopropanecarboxamide (40 mg, 0.11 mmol) was suspended in about 1 mL of acetone. Ethanesulfonic acid (0.22 mmol, 24.5 mg) was added and the resulting suspension stirred for one day. The solids were isolated by centrifugation.

The physical properties of the hydrate of the di-esylate salt of (R)—N-(4-(3-aminopiperidin-1-yl)-5-bromo-1H-pyrrolo[2,3-b]pyridin-3-yl)cyclopropanecarboxamide are summarized in Table 40 below.

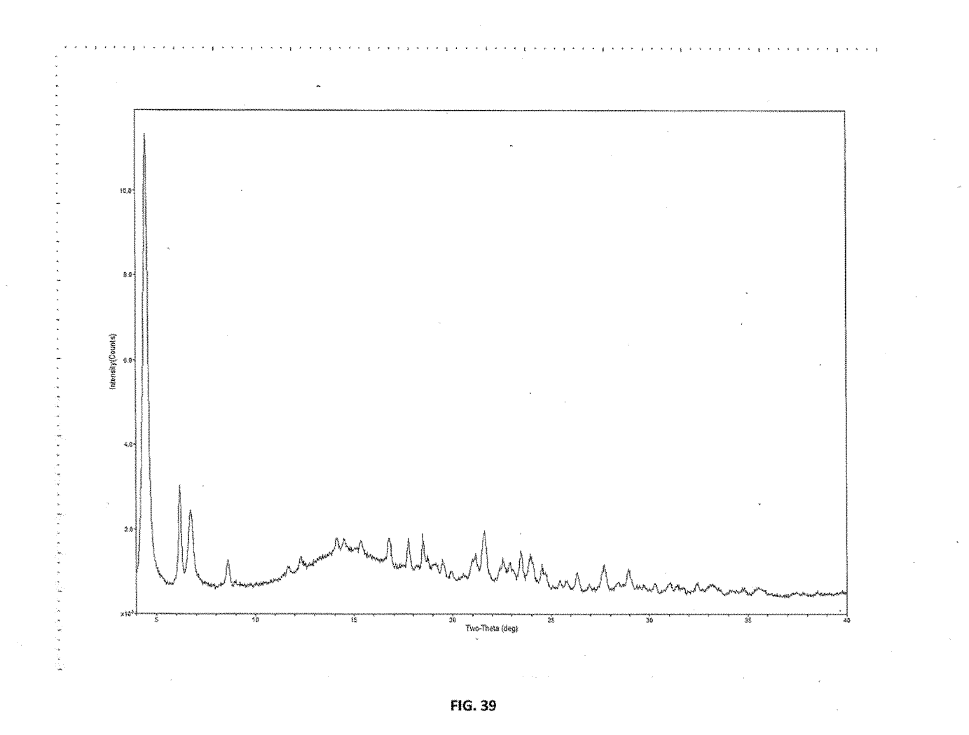

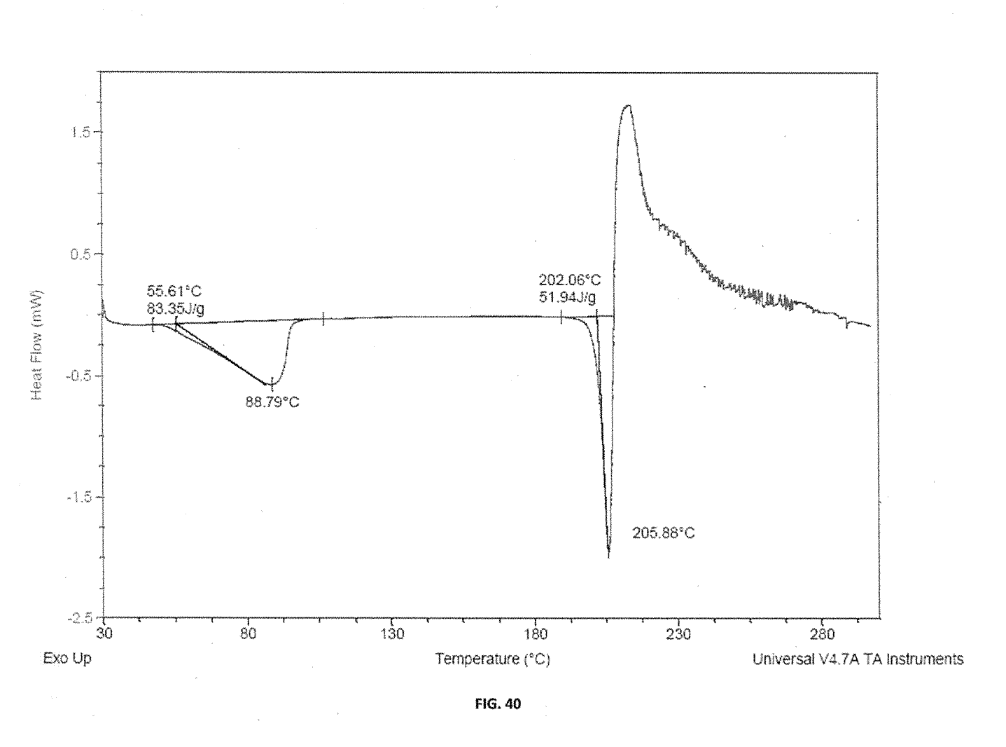

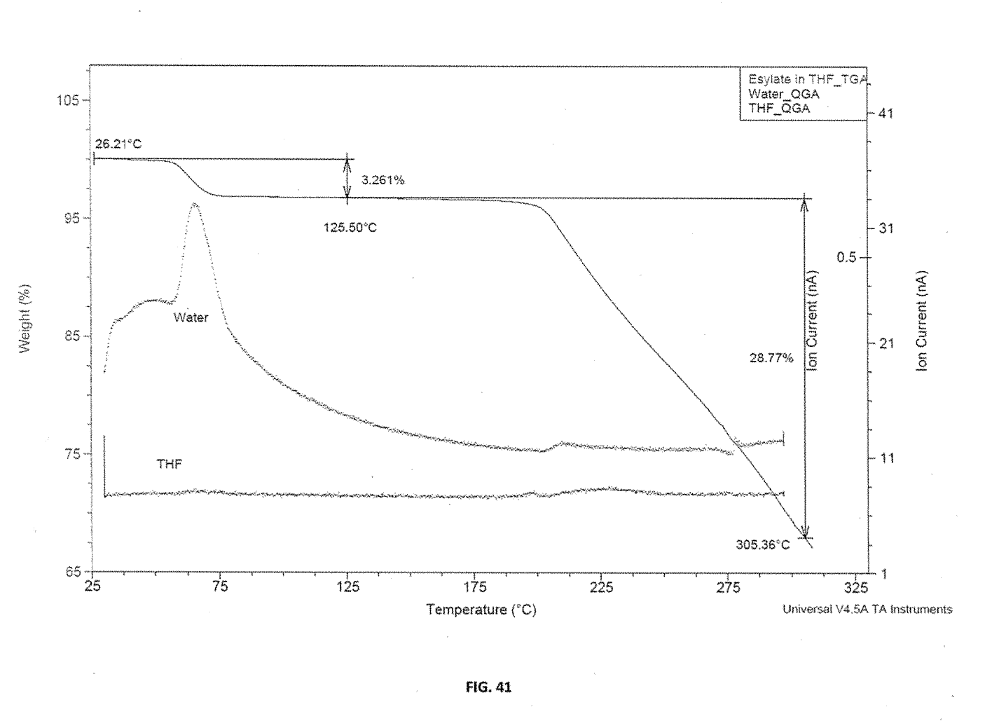

TABLE 40

| | |
|---|---|
| XRPD | FIG. 39: Peaks of ≥5% relative height at about 4.5, |
| | 6.2, 6.7, 8.6, 16.8, 17.8, 18.5, 21.6, 23.5, 24.0, and |
| | 27.7 °2θ. |
| DSC | FIG. 40: Endotherm with extrapolated onset temperature |
| | about 56° C. and enthalpy of fusion about 83 J/g. |
| | Endotherm with extrapolated onset temperature about |
| | 202° C. and enthalpy of fusion about 52 J/g. |
| TGA | FIG. 41: About 3.3% weight loss up to about 126° C. |

Certain XRPD diffraction peaks for the of (R)—N-(4-(3-aminopiperidin-1-yl)-5-bromo-1H-pyrrolo[2,3-b]pyridin-3-yl)cyclopropanecarboxamide are shown in Table 41 below.

TABLE 41
SCAN: 4.0/40.0055/0.01975/21.6(sec), Cu(40 kV, 40 mA),
I(p) = 11324, 09/08/13 05:40p
PEAK: 19(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 4.514 | 19.5616 | 914 | 10410 | 100.0 | 167672 | 100.0 | 0.270 |
| 2 | 6.211 | 14.2195 | 736 | 2294 | 22.0 | 22548 | 13.4 | 0.165 |
| 3 | 6.746 | 13.0930 | 698 | 1761 | 16.9 | 34370 | 20.5 | 0.328 |
| 4 | 8.622 | 10.2473 | 654 | 627 | 6.0 | 8284 | 4.9 | 0.222 |
| 5 | 9.010 | 9.8065 | 658 | 137 | 1.3 | 1695 | 1.0 | 0.207 |
| 6 | 11.684 | 7.5680 | 932 | 201 | 1.9 | 1722 | 1.0 | 0.144 |
| 7 | 12.335 | 7.1697 | 1077 | 282 | 2.7 | 4733 | 2.8 | 0.282 |
| 8 | 14.151 | 6.2534 | 1408 | 397 | 3.8 | 8856 | 5.3 | 0.374 |
| 9 | 14.492 | 6.1072 | 1469 | 312 | 3.0 | 5288 | 3.2 | 0.284 |
| 10 | 16.799 | 5.2732 | 1192 | 606 | 5.8 | 5943 | 3.5 | 0.165 |
| 11 | 17.785 | 4.9831 | 1076 | 711 | 6.8 | 6147 | 3.7 | 0.145 |
| 12 | 18.518 | 4.7874 | 1037 | 862 | 8.3 | 12719 | 7.6 | 0.248 |
| 13 | 18.772 | 4.7233 | 984 | 403 | 3.9 | 13963 | 8.3 | 0.581 |
| 14 | 19.205 | 4.6178 | 984 | 230 | 2.2 | 3208 | 1.9 | 0.234 |
| 15 | 19.528 | 4.5421 | 964 | 332 | 3.2 | 3466 | 2.1 | 0.176 |
| 16 | 21.185 | 4.1905 | 810 | 628 | 6.0 | 15192 | 9.1 | 0.406 |
| 17 | 21.638 | 4.1038 | 834 | 1141 | 11.0 | 17589 | 10.5 | 0.259 |
| 18 | 22.588 | 3.9333 | 839 | 490 | 4.7 | 7497 | 4.5 | 0.257 |
| 19 | 22.975 | 3.8679 | 860 | 360 | 3.5 | 7248 | 4.3 | 0.338 |
| 20 | 23.496 | 3.7832 | 867 | 642 | 6.2 | 5928 | 3.5 | 0.155 |
| 21 | 23.989 | 3.7066 | 847 | 583 | 5.6 | 6870 | 4.1 | 0.198 |
| 22 | 24.561 | 3.6215 | 723 | 461 | 4.4 | 5415 | 3.2 | 0.197 |
| 23 | 25.484 | 3.4924 | 617 | 176 | 1.7 | 1833 | 1.1 | 0.174 |
| 24 | 25.843 | 3.4447 | 585 | 211 | 2.0 | 3940 | 2.3 | 0.313 |
| 25 | 26.375 | 3.3765 | 575 | 409 | 3.9 | 4348 | 2.6 | 0.178 |
| 26 | 26.972 | 3.3030 | 569 | 141 | 1.4 | 1036 | 0.6 | 0.123 |
| 27 | 27.721 | 3.2154 | 595 | 575 | 5.5 | 7815 | 4.7 | 0.228 |
| 28 | 28.432 | 3.1366 | 622 | 130 | 1.3 | 2170 | 1.3 | 0.280 |
| 29 | 28.965 | 3.0802 | 600 | 478 | 4.6 | 9430 | 5.6 | 0.331 |
| 30 | 30.306 | 2.9468 | 539 | 184 | 1.8 | 1499 | 0.9 | 0.137 |
| 31 | 31.077 | 2.8755 | 511 | 251 | 2.4 | 4379 | 2.6 | 0.293 |
| 32 | 31.471 | 2.8404 | 513 | 192 | 1.8 | 3762 | 2.2 | 0.328 |
| 33 | 31.789 | 2.8126 | 524 | 113 | 1.1 | 1308 | 0.8 | 0.194 |
| 34 | 32.495 | 2.7532 | 542 | 210 | 2.0 | 2217 | 1.3 | 0.178 |
| 35 | 33.212 | 2.6953 | 526 | 197 | 1.9 | 4634 | 2.8 | 0.395 |
| 36 | 33.642 | 2.6619 | 489 | 97 | 0.9 | 2297 | 1.4 | 0.397 |
| 37 | 34.238 | 2.6168 | 474 | 99 | 1.0 | 2432 | 1.5 | 0.411 |
| 38 | 34.755 | 2.5791 | 478 | 160 | 1.5 | 3467 | 2.1 | 0.364 |
| 39 | 35.485 | 2.5277 | 473 | 169 | 1.6 | 4029 | 2.4 | 0.400 |
| 40 | 35.838 | 2.5036 | 456 | 113 | 1.1 | 4601 | 2.7 | 0.686 |
| 41 | 37.492 | 2.3969 | 447 | 84 | 0.8 | 1348 | 0.8 | 0.269 |

NOTE:
Intensity = Counts, 2T(0) = 0.0(deg), Wavelength to Compute d-Spacing = 1.54059 Å (Cu/K-alpha1)

### Example 22

(R)—N-(4-(3-aminopiperidin-1-yl)-5-bromo-1H-pyrrolo[2,3-b]pyridin-3-yl)cyclopropanecarboxamide mono-methanesulfonic acid salt: Approximately 5.0 g of (R)—N-(4-(3-aminopiperidin-1-yl)-5-bromo-1H-pyrrolo[2,3-b]pyridin-3-yl)cyclopropanecarboxamide was weighed into a 500 mL of round-bottom flask, and then approximately 250 mL of acetone was added. 1.0 equivalents of methanesulfonic acid was slowly titrated into the reaction bulb. The suspension was kept stirring on a magnetic stirrer at room temperature. After 24 hrs, the remaining solid was separated by vacuum filtration. The wet cake was dried under reduced pressure at 35° C. overnight. The physical properties of the non-solvated mono-mesylate salt of (R)—N-(4-(3-aminopiperidin-1-yl)-5-bromo-1H-pyrrolo[2,3-b]pyridin-3-yl)cyclopropanecarboxamide are summarized in Table 42 below.

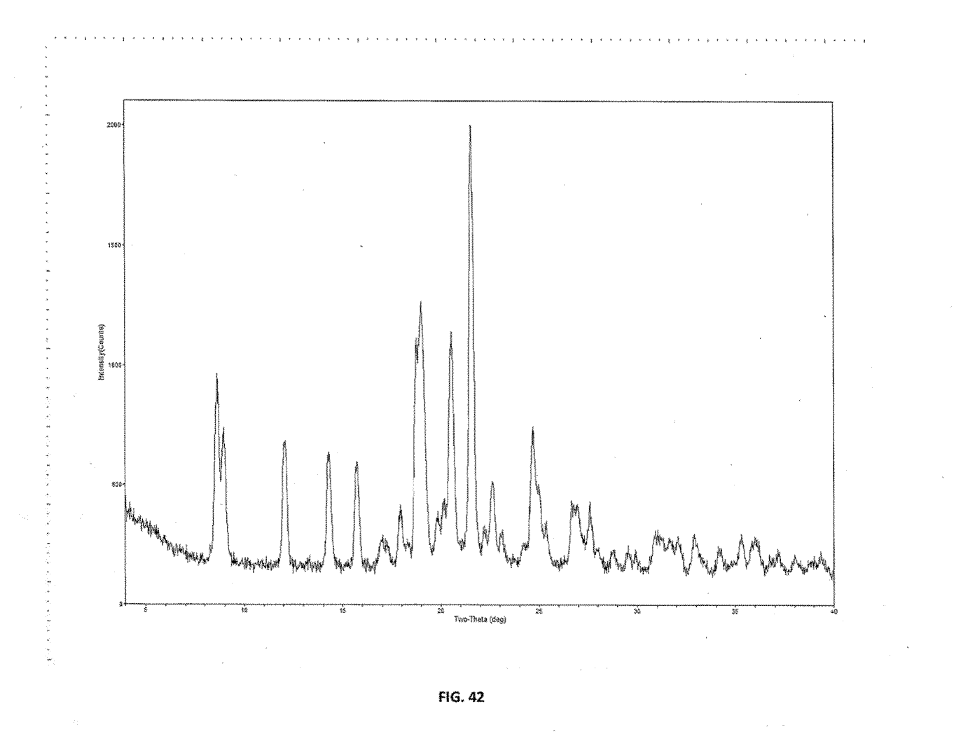

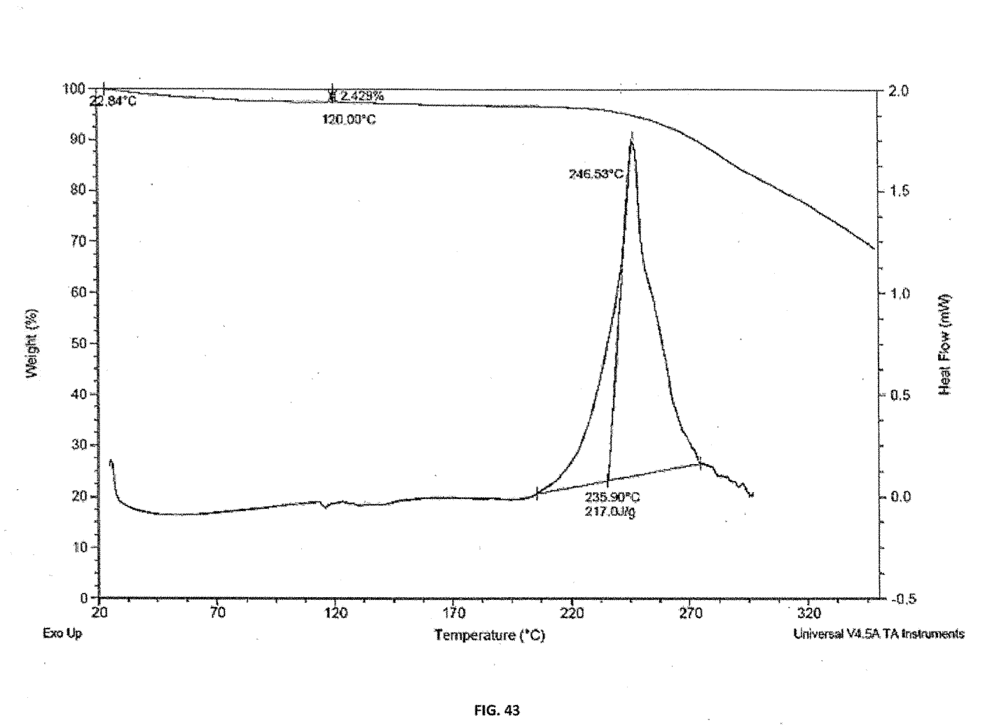

TABLE 42

| | |
|---|---|
| XRPD | FIG. 42: Peaks of ≥15% relative height at about 8.7, |
| | 9.0, 12.1, 14.3, 15.8, 19.0, 20.6, 21.6, 22.7, 24.7, |
| | 26.7, and 27.6 °2θ. |
| DSC | FIG. 43: Exotherm with extrapolated onset temperature |
| | about 236° C. and enthalpy of fusion about 217 J/g. |
| TGA | FIG. 43: About 2.4% weight loss up to about 120° C. |

Certain XRPD diffraction peaks for the non-solvated mono-mesylate salt of (R)—N-(4-(3-aminopiperidin-1-yl)-5-bromo-1H-pyrrolo[2,3-b]pyridin-3-yl)cyclopropanecarboxamide are shown in Table 43 below.

TABLE 43
SCAN: 4.0/40.0087/0.01972/18.6(sec), Cu(40 kV, 40 mA),
I(p) = 2002, 09/11/14 11:24p
PEAK: 27(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 8.654 | 10.2098 | 186 | 776 | 44.2 | 13180 | 48.7 | 0.285 |
| 2 | 8.988 | 9.8305 | 172 | 567 | 32.3 | 12995 | 48.0 | 0.384 |
| 3 | 12.124 | 7.2939 | 167 | 518 | 29.5 | 7254 | 26.8 | 0.235 |
| 4 | 14.313 | 6.1829 | 158 | 479 | 27.3 | 6889 | 25.5 | 0.241 |
| 5 | 15.773 | 5.6140 | 162 | 437 | 24.9 | 5981 | 22.1 | 0.229 |
| 6 | 17.037 | 5.2002 | 174 | 109 | 6.2 | 2119 | 7.8 | 0.325 |
| 7 | 17.960 | 4.9348 | 202 | 216 | 12.3 | 2694 | 10.0 | 0.209 |
| 8 | 19.046 | 4.6559 | 230 | 1034 | 58.9 | 27057 | 100.0 | 0.438 |
| 9 | 19.892 | 4.4597 | 238 | 153 | 8.7 | 2229 | 8.2 | 0.244 |
| 10 | 20.565 | 4.3154 | 246 | 894 | 50.9 | 16212 | 59.9 | 0.304 |
| 11 | 21.571 | 4.1163 | 246 | 1756 | 100.0 | 24154 | 89.3 | 0.231 |
| 12 | 22.260 | 3.9905 | 236 | 95 | 5.4 | 1174 | 4.3 | 0.207 |
| 13 | 22.656 | 3.9216 | 217 | 298 | 17.0 | 5881 | 21.7 | 0.330 |
| 14 | 23.166 | 3.8364 | 237 | 78 | 4.4 | 340 | 1.3 | 0.073 |
| 15 | 24.725 | 3.5978 | 175 | 571 | 32.5 | 15604 | 57.7 | 0.458 |
| 16 | 25.394 | 3.5046 | 197 | 154 | 8.7 | 2404 | 8.9 | 0.262 |
| 17 | 26.699 | 3.3361 | 167 | 268 | 15.3 | 8017 | 29.6 | 0.501 |
| 18 | 27.623 | 3.2267 | 165 | 266 | 15.1 | 4593 | 17.0 | 0.289 |
| 19 | 28.056 | 3.1778 | 165 | 78 | 4.4 | 1070 | 4.0 | 0.230 |
| 20 | 28.826 | 3.0947 | 159 | 75 | 4.3 | 949 | 3.5 | 0.212 |
| 21 | 29.559 | 3.0196 | 157 | 93 | 5.3 | 1439 | 5.3 | 0.260 |
| 22 | 29.949 | 2.9811 | 151 | 82 | 4.7 | 1178 | 4.4 | 0.241 |
| 23 | 30.958 | 2.8863 | 150 | 160 | 9.1 | 6850 | 25.3 | 0.719 |
| 24 | 31.648 | 2.8248 | 159 | 124 | 7.1 | 8032 | 29.7 | 1.084 |
| 25 | 32.102 | 2.7860 | 150 | 139 | 7.9 | 2200 | 8.1 | 0.266 |
| 26 | 32.929 | 2.7179 | 148 | 150 | 8.5 | 2834 | 10.5 | 0.317 |
| 27 | 34.136 | 2.6244 | 150 | 96 | 5.4 | 1110 | 4.1 | 0.195 |
| 28 | 35.336 | 2.5381 | 174 | 126 | 7.2 | 1549 | 5.7 | 0.205 |
| 29 | 36.027 | 2.4909 | 165 | 124 | 7.1 | 2513 | 9.3 | 0.339 |
| 30 | 37.174 | 2.4166 | 150 | 85 | 4.8 | 1424 | 5.3 | 0.280 |
| 31 | 38.040 | 2.3636 | 148 | 63 | 3.6 | 712 | 2.6 | 0.190 |
| 32 | 38.924 | 2.3119 | 142 | 46 | 2.6 | 2074 | 7.7 | 0.757 |
| 33 | 39.360 | 2.2873 | 137 | 89 | 5.1 | 2074 | 7.7 | 0.392 |

NOTE:
Intensity = Counts, 2T(0) = 0.0(deg), Wavelength to Compute d-Spacing = 1.54059 Å (Cu/K-alpha1)

### Example 23

(R)—N-(4-(3-aminopiperidin-1-yl)-5-bromo-1H-pyrrolo[2,3-b]pyridin-3-yl)cyclopropanecarboxamide mono-ethanesulfonic acid salt: Approximately 10 g of (R)—N-(4-(3-aminopiperidin-1-yl)-5-bromo-1H-pyrrolo[2,3-b]pyridin-3-yl)cyclopropanecarboxamide was weighed into a 500 mL round-bottom flask, and then approximately 500 mL of acetone was added. 1.0 equivalents of ethanesulfonic acid was slowly titrated into the sample. The suspension was kept stirring on a magnetic stirrer at room temperature. After 24 hrs, the remaining solid was isolated by vacuum filtering. After that, the wet cake was suspended with 180 mL acetone for purification. The purified solid samples were isolated by vacuum filtering and dried under reduced pressure at room temperature. The physical properties of the non-solvated mono-esylate salt of (R)—N-(4-(3-aminopiperidin-1-yl)-5-bromo-1H-pyrrolo[2,3-b]pyridin-3-yl)cyclopropanecarboxamide are summarized in Table 44 below.

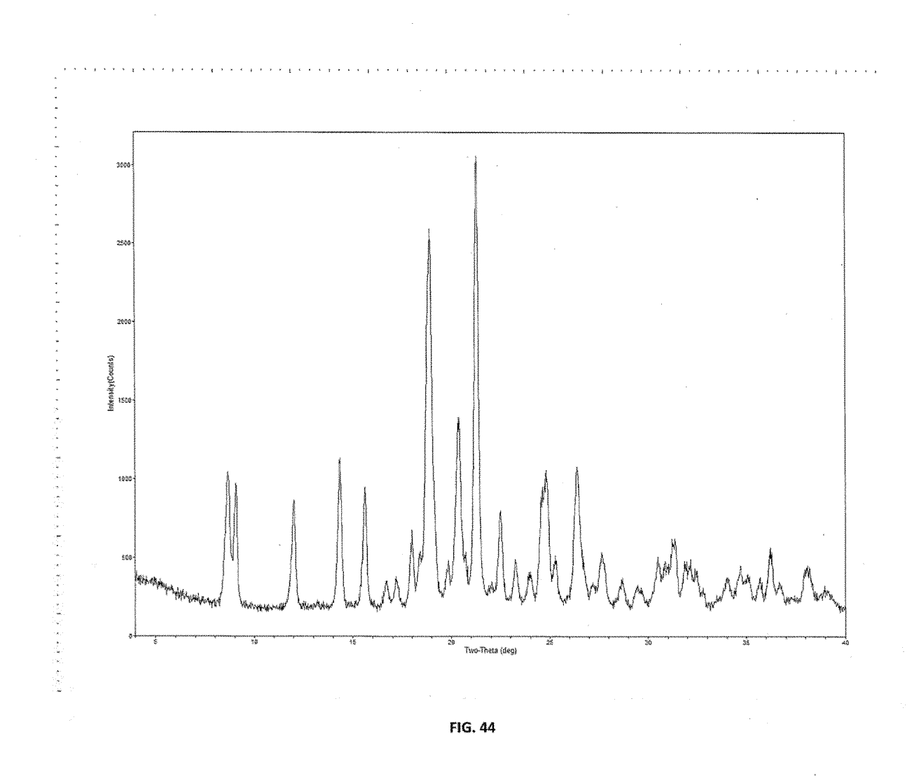

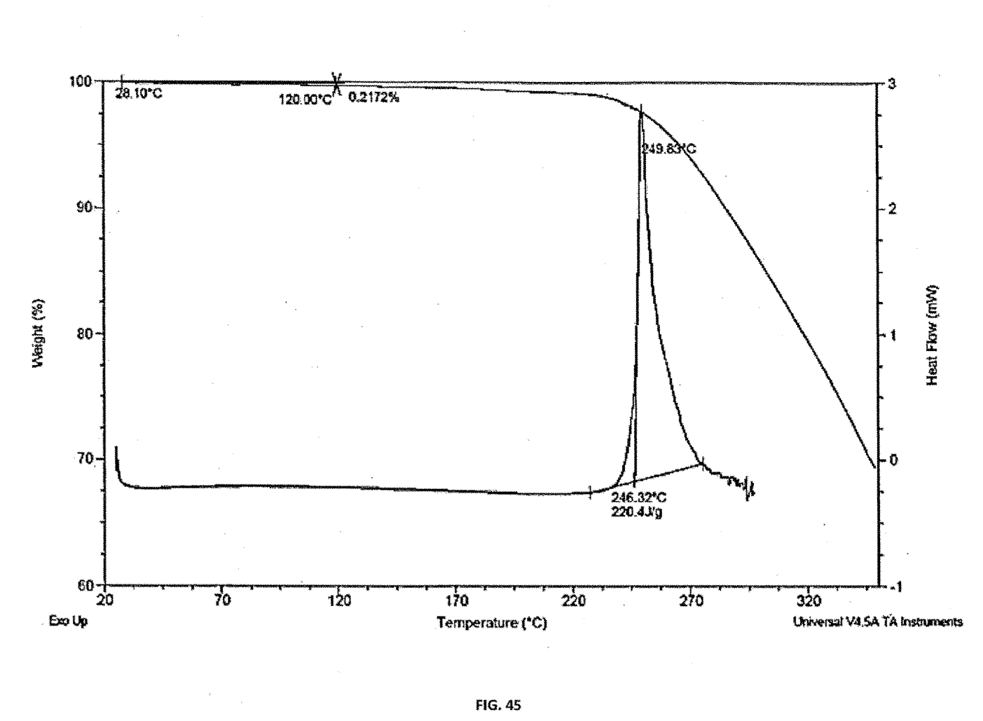

TABLE 44

| | |
|---|---|
| XRPD | FIG. 44: Peaks of ≥15% relative height at about 8.7, |
| | 9.1, 12.1, 14.4, 15.7, 18.0, 19.0, 20.4, 21.3, 22.5, |
| | 29.5, and 26.4, °2θ. |
| DSC | FIG. 45: Exotherm with extrapolated onset temperature |
| | about 246° C. and enthalpy of fusion about 220 J/g. |
| TGA | FIG. 45: About 0.2% weight loss up to about 120° C. |

Certain XRPD diffraction peaks for the non-solvated mono-esylate salt of (R)—N-(4-(3-aminopiperidin-1-yl)-5-bromo-1H-pyrrolo[2,3-b]pyridin-3-yl)cyclopropanecarboxamide are shown in Table 45 below.

TABLE 45
SCAN: 4.0/40.0102/0.01973/18.6(sec), Cu(40 kV, 40 mA),
I(p) = 3055, 11/28/13 12:26a
PEAK: 23(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 8.677 | 10.1827 | 206 | 835 | 30.6 | 16425 | 33.5 | 0.330 |
| 2 | 9.074 | 9.7383 | 201 | 771 | 28.2 | 11030 | 22.5 | 0.240 |
| 3 | 12.050 | 7.3389 | 186 | 679 | 24.9 | 8943 | 18.2 | 0.221 |
| 4 | 14.377 | 6.1559 | 195 | 936 | 34.3 | 11306 | 23.0 | 0.203 |
| 5 | 15.660 | 5.6542 | 199 | 749 | 27.4 | 9346 | 19.0 | 0.209 |
| 6 | 16.743 | 5.2908 | 198 | 158 | 5.8 | 2278 | 4.6 | 0.241 |
| 7 | 17.203 | 5.1503 | 205 | 174 | 6.4 | 2423 | 4.9 | 0.233 |
| 8 | 18.028 | 4.9164 | 212 | 463 | 17.0 | 4943 | 10.1 | 0.179 |
| 9 | 18.443 | 4.8069 | 249 | 293 | 10.7 | 9172 | 18.7 | 0.524 |
| 10 | 18.957 | 4.6775 | 249 | 2345 | 85.9 | 49093 | 100.0 | 0.351 |
| 11 | 19.883 | 4.4618 | 249 | 236 | 8.7 | 2266 | 4.6 | 0.161 |
| 12 | 20.398 | 4.3504 | 324 | 1066 | 39.0 | 17646 | 35.9 | 0.278 |
| 13 | 21.325 | 4.1633 | 323 | 2732 | 100.0 | 34862 | 71.0 | 0.214 |
| 14 | 22.546 | 3.9404 | 254 | 550 | 20.1 | 7406 | 15.1 | 0.226 |
| 15 | 23.281 | 3.8177 | 237 | 256 | 9.4 | 2976 | 6.1 | 0.195 |
| 16 | 24.046 | 3.6980 | 256 | 156 | 5.7 | 1716 | 3.5 | 0.184 |
| 17 | 24.837 | 3.5820 | 250 | 805 | 29.5 | 19930 | 40.6 | 0.415 |
| 18 | 25.310 | 3.5160 | 237 | 277 | 10.1 | 4634 | 9.4 | 0.281 |
| 19 | 26.416 | 3.3713 | 252 | 823 | 30.1 | 16123 | 32.8 | 0.328 |
| 20 | 27.226 | 3.2728 | 263 | 83 | 3.0 | 1505 | 3.1 | 0.304 |
| 21 | 27.678 | 3.2203 | 238 | 298 | 10.9 | 5718 | 11.6 | 0.322 |
| 22 | 28.724 | 3.1054 | 200 | 173 | 6.3 | 2684 | 5.5 | 0.261 |
| 23 | 29.453 | 3.0302 | 201 | 120 | 4.4 | 2522 | 5.1 | 0.352 |
| 24 | 30.520 | 2.9267 | 212 | 293 | 10.7 | 4356 | 8.9 | 0.249 |
| 25 | 30.893 | 2.8922 | 248 | 229 | 8.4 | 8266 | 16.8 | 0.606 |
| 26 | 31.388 | 2.8477 | 248 | 373 | 13.6 | 10107 | 20.6 | 0.455 |
| 27 | 31.882 | 2.8046 | 284 | 196 | 7.2 | 3215 | 6.5 | 0.275 |
| 28 | 32.438 | 2.7579 | 240 | 195 | 7.1 | 4840 | 9.9 | 0.416 |
| 29 | 34.050 | 2.6309 | 251 | 128 | 4.7 | 2007 | 4.1 | 0.262 |
| 30 | 34.723 | 2.5814 | 247 | 205 | 7.5 | 5423 | 11.0 | 0.443 |
| 31 | 35.096 | 2.5548 | 263 | 135 | 4.9 | 3384 | 6.9 | 0.421 |
| 32 | 35.726 | 2.5112 | 253 | 127 | 4.6 | 1145 | 2.3 | 0.152 |
| 33 | 36.242 | 2.4766 | 248 | 319 | 11.7 | 5357 | 10.9 | 0.282 |
| 34 | 36.680 | 2.4481 | 236 | 117 | 4.3 | 1846 | 3.8 | 0.264 |
| 35 | 37.996 | 2.3662 | 243 | 187 | 6.8 | 4435 | 9.0 | 0.397 |
| 36 | 39.048 | 2.3049 | 228 | 73 | 2.7 | 1814 | 3.7 | 0.416 |

NOTE:
Intensity = Counts, 2T(0) = 0.0(deg), Wavelength to Compute d-Spacing = 1.54059 Å (Cu/K-alpha1)

### Example 24 (R)—N-(4-(3-aminopiperidin-1-yl)-5-bromo-1H-pyrrolo[2,3-b]pyridin-3-yl)cyclopropanecarboxamide di-benzenesulfonic acid salt

Method A: Approximately 40 mg of (R)—N-(4-(3-aminopiperidin-1-yl)-5-bromo-1H-pyrrolo[2,3-b]pyridin-3-yl)cyclopropanecarboxamide was weighed into a 4 mL vial and suspended with suitable amount of THF, then 2 equivalents of benzenesulfonic acid was added. The mixture was stirred for 1 day.

Method B: (R)—N-(4-(3-aminopiperidin-1-yl)-5-bromo-1H-pyrrolo[2,3-b]pyridin-3-yl)cyclopropanecarboxamide (40 mg) was weighed into a 4 mL centrifuge tube and added into 2 mL of THF to form a suspension. An appropriate amount of benzenesulfonic acid (39 mg, content 90%) was added into the suspension to keep the molar ratio of API:acid equal to 1:2.1. The suspension was mixed completely on a rotary shaker for 1 day at room temperature. The precipitation was centrifuged at 10,000 rpm for 3 minutes, and dried under reduced pressure for 1 day at room temperature.

The physical properties of the non-solvated di-besylate salt of (R)—N-(4-(3-aminopiperidin-1-yl)-5-bromo-1H-pyrrolo[2,3-b]pyridin-3-yl)cyclopropanecarboxamide are summarized in Table 46 below.

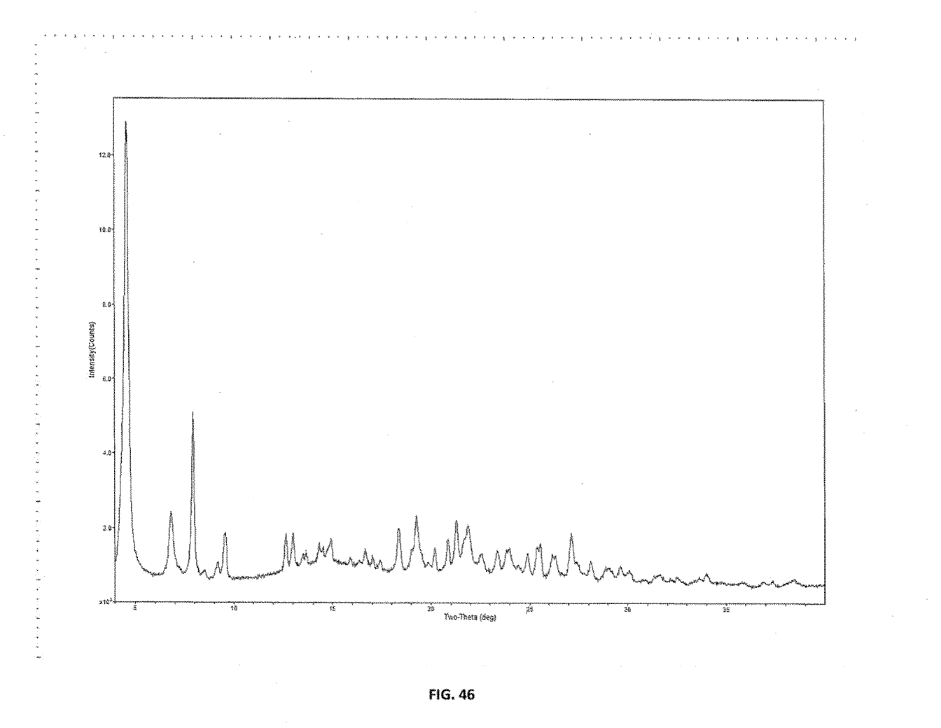

TABLE 46

| | |
|---|---|
| XRPD | FIG. 46: Peaks of ≥5% relative height at about 4.6, |
| | 6.8, 8.0, 9.6, 13.0, 14.9, 18.4, 19.3, 20.9, 21.3, 21.9, |
| | 25.6, and 27.2 °2θ. |

Certain XRPD diffraction peaks for the non-solvated di-besylate salt of (R)—N-(4-(3-aminopiperidin-1-yl)-5-bromo-1H-pyrrolo[2,3-b]pyridin-3-yl)cyclopropanecarboxamide are shown in Table 47 below.

TABLE 47
SCAN: 4.0/39.9975/0.01975/21.6(sec), Cu(40 kV,40 mA),
I(p) = 12887, 09/10/13 05:02p
PEAK: 21(pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 4.631 | 19.0640 | 992 | 11895 | 100.0 | 186126 | 100.0 | 0.263 |
| 2 | 6.843 | 12.9061 | 789 | 1639 | 13.8 | 24874 | 13.4 | 0.255 |
| 3 | 7.968 | 11.0866 | 747 | 4350 | 36.6 | 41038 | 22.0 | 0.158 |
| 4 | 9.193 | 9.6124 | 656 | 435 | 3.7 | 6144 | 3.3 | 0.237 |
| 5 | 9.587 | 9.2176 | 655 | 1232 | 10.4 | 13993 | 7.5 | 0.191 |
| 6 | 13.040 | 6.7836 | 968 | 900 | 7.6 | 8108 | 4.4 | 0.151 |
| 7 | 13.532 | 6.5381 | 1038 | 283 | 2.4 | 2873 | 1.5 | 0.170 |
| 8 | 14.346 | 6.1688 | 1066 | 550 | 4.6 | 10814 | 5.8 | 0.330 |
| 9 | 14.923 | 5.9319 | 1055 | 679 | 5.7 | 13008 | 7.0 | 0.322 |
| 10 | 16.370 | 5.4107 | 1031 | 126 | 1.1 | 1528 | 0.8 | 0.203 |
| 11 | 16.696 | 5.3055 | 987 | 461 | 3.9 | 6064 | 3.3 | 0.221 |
| 12 | 17.038 | 5.2000 | 955 | 324 | 2.7 | 4015 | 2.2 | 0.208 |
| 13 | 18.376 | 4.8243 | 907 | 1090 | 9.2 | 12376 | 6.6 | 0.191 |
| 14 | 19.285 | 4.5939 | 986 | 1352 | 11.4 | 21425 | 11.5 | 0.266 |
| 15 | 20.231 | 4.3858 | 991 | 500 | 4.2 | 2760 | 1.5 | 0.093 |
| 16 | 20.899 | 4.2472 | 1060 | 658 | 5.5 | 4575 | 2.5 | 0.117 |
| 17 | 21.316 | 4.1650 | 992 | 1219 | 10.3 | 16320 | 8.8 | 0.225 |
| 18 | 21.909 | 4.0535 | 1006 | 1081 | 9.1 | 26070 | 14.0 | 0.405 |
| 19 | 22.603 | 3.9307 | 1008 | 331 | 2.8 | 3437 | 1.8 | 0.174 |
| 20 | 23.392 | 3.7999 | 946 | 480 | 4.0 | 4252 | 2.3 | 0.149 |
| 21 | 23.984 | 3.7073 | 870 | 583 | 4.9 | 14238 | 7.6 | 0.410 |
| 22 | 24.441 | 3.6391 | 891 | 134 | 1.1 | 2320 | 1.2 | 0.291 |
| 23 | 24.929 | 3.5689 | 841 | 511 | 4.3 | 4336 | 2.3 | 0.142 |
| 24 | 25.563 | 3.4819 | 778 | 801 | 6.7 | 12001 | 6.4 | 0.252 |
| 25 | 26.214 | 3.3969 | 759 | 526 | 4.4 | 10023 | 5.4 | 0.320 |
| 26 | 27.163 | 3.2802 | 773 | 1108 | 9.3 | 17128 | 9.2 | 0.259 |
| 27 | 27.459 | 3.2456 | 726 | 391 | 3.3 | 12046 | 6.5 | 0.517 |
| 28 | 28.149 | 3.1675 | 728 | 409 | 3.4 | 4000 | 2.1 | 0.164 |
| 29 | 28.902 | 3.0867 | 694 | 304 | 2.6 | 5259 | 2.8 | 0.290 |
| 30 | 29.650 | 3.0105 | 649 | 358 | 3.0 | 8965 | 4.8 | 0.421 |
| 31 | 30.120 | 2.9646 | 662 | 252 | 2.1 | 4560 | 2.4 | 0.304 |
| 32 | 31.646 | 2.8250 | 584 | 209 | 1.8 | 4984 | 2.7 | 0.400 |
| 33 | 32.183 | 2.7792 | 556 | 137 | 1.2 | 3653 | 2.0 | 0.448 |
| 34 | 32.593 | 2.7451 | 581 | 145 | 1.2 | 1516 | 0.8 | 0.175 |
| 35 | 33.660 | 2.6605 | 536 | 185 | 1.6 | 4939 | 2.7 | 0.449 |
| 36 | 34.035 | 2.6320 | 519 | 304 | 2.6 | 9050 | 4.9 | 0.500 |
| 37 | 35.832 | 2.5041 | 486 | 117 | 1.0 | 2354 | 1.3 | 0.337 |
| 38 | 36.954 | 2.4305 | 473 | 129 | 1.1 | 3136 | 1.7 | 0.408 |
| 39 | 37.388 | 2.4033 | 476 | 162 | 1.4 | 3179 | 1.7 | 0.330 |
| 40 | 38.478 | 2.3377 | 479 | 197 | 1.7 | 4907 | 2.6 | 0.417 |

NOTE:
Intensity = Counts, 2T(0) = 0.0(deg), Wavelength to Compute d-Spacing = 1.54059 Å (Cu/K-alpha1)

### Example 25 (R)—N-(4-(3-aminopiperidin-1-yl)-5-bromo-1H-pyrrolo[2,3-b]pyridin-3-yl)cyclopropanecarboxamide di-toluenesulfonic acid salt

Method A: Approximately 40 mg of (R)—N-(4-(3-aminopiperidin-1-yl)-5-bromo-1H-pyrrolo[2,3-b]pyridin-3-yl)cyclopropanecarboxamide was weighed into a 4 mL vial and suspended with suitable amount of acetone or THF, then 2 equivalents of toluenesulfonic acid was added and the mixture was stirred for 1 day.

Method B: (R)—N-(4-(3-aminopiperidin-1-yl)-5-bromo-1H-pyrrolo[2,3-b]pyridin-3-yl)cyclopropanecarboxamide (40 mg) was weighed into a 4 mL centrifuge tube and added into 2 mL of IPA (isopropanol) to form a suspension. An appropriate amount of toluenesulfonic acid monohydrate (43 mg, content 99%) was added into the suspension to keep the molar ratio of API:acid equal to 1:2.1. The suspension was mixed completely on a rotary shaker for 1 day at room temperature. The precipitation were centrifuged at 10,000 rpm for 3 minutes, and dried under reduced pressure for 1 day at room temperature.

The physical properties of the non-solvated di tosylate salt of (R)—N-(4-(3-aminopiperidin-1-yl)-5-bromo-1H-pyrrolo[2,3-b]pyridin-3-yl)cyclopropanecarboxamide are summarized in Table 48 below.

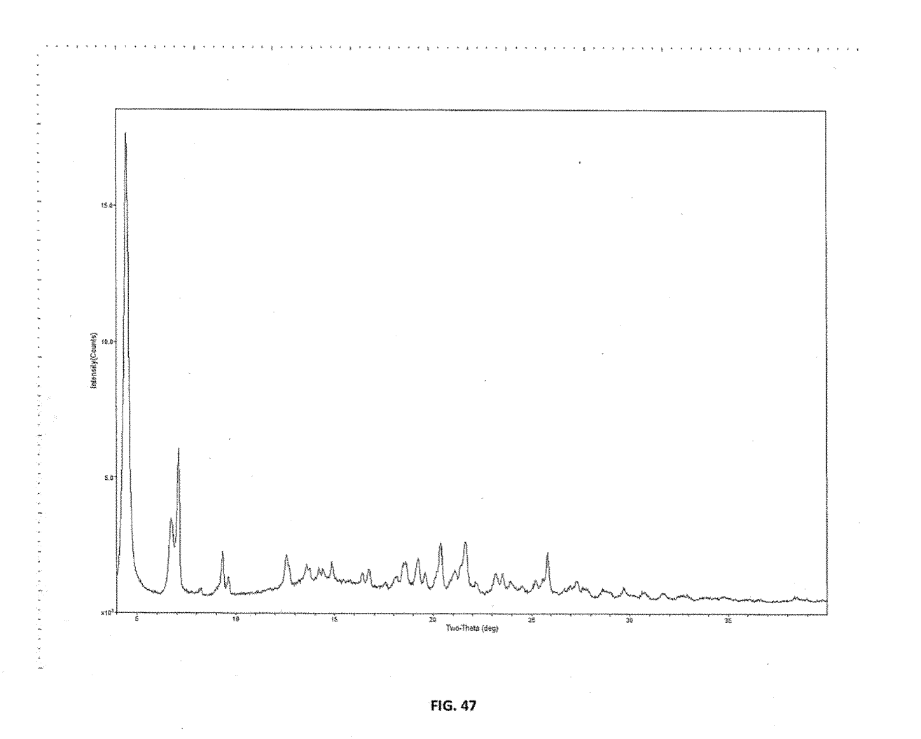

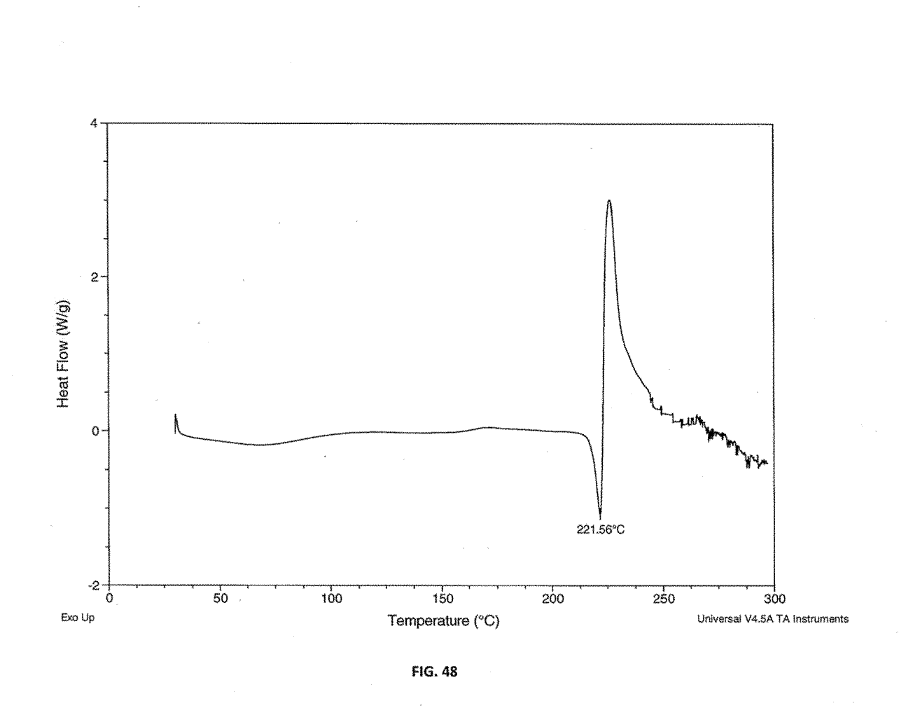

TABLE 48

| | |
|---|---|
| XRPD | FIG. 47: Peaks of ≥5% relative height at about 4.5, |
| | 6.7, 7.1, 9.4, 12.6, 19.3, 20.4, 21.6, and 25.9 °2θ. |
| DSC | FIG. 48: Endotherm at about 222° C. |

Certain XRPD diffraction peaks for the non-solvated di tosylate salt of (R)—N-(4-(3-aminopiperidin-1-yl)-5-bromo-1H-pyrrolo[2,3-b]pyridin-3-yl)cyclopropanecarboxamide are shown in Table 49 below.

TABLE 49
SCAN: 4.0/39.9975/0.01975/21.6(sec), Cu(40 kV, 40 mA),
I(p) = 17690, 09/10/13 05:02p
PEAK: 21 (pts)/Parabolic Filter, Threshold = 3.0,
Cutoff = 0.1%, BG = 3/1.0, Peak-Top = Summit

| # | 2-Theta | d(Å) | BG | Height | H % | Area | A % | FWHM |
|---|---|---|---|---|---|---|---|---|
| 1 | 4.533 | 19.4790 | 886 | 16804 | 100.0 | 279482 | 100.0 | 0.279 |
| 2 | 6.746 | 13.0921 | 756 | 2740 | 16.3 | 71478 | 25.6 | 0.438 |
| 3 | 7.139 | 12.3721 | 747 | 5314 | 31.6 | 64806 | 23.2 | 0.205 |
| 4 | 8.246 | 10.7132 | 714 | 226 | 1.3 | 1568 | 0.6 | 0.116 |
| 5 | 9.352 | 9.4492 | 699 | 1560 | 9.3 | 21039 | 7.5 | 0.226 |
| 6 | 9.645 | 9.1626 | 710 | 641 | 3.8 | 6977 | 2.5 | 0.183 |
| 7 | 12.609 | 7.0148 | 1042 | 1113 | 6.6 | 14355 | 5.1 | 0.216 |
| 8 | 13.616 | 6.4983 | 1217 | 602 | 3.6 | 8832 | 3.2 | 0.246 |
| 9 | 14.214 | 6.2258 | 1220 | 476 | 2.8 | 7468 | 2.7 | 0.263 |
| 10 | 14.882 | 5.9478 | 1223 | 685 | 4.1 | 9425 | 3.4 | 0.231 |
| 11 | 16.461 | 5.3808 | 1103 | 411 | 2.4 | 3980 | 1.4 | 0.163 |
| 12 | 16.792 | 5.2754 | 1069 | 579 | 3.4 | 5092 | 1.8 | 0.148 |
| 13 | 17.598 | 5.0356 | 1017 | 167 | 1.0 | 758 | 0.3 | 0.076 |
| 14 | 18.179 | 4.8761 | 1038 | 353 | 2.1 | 6059 | 2.2 | 0.288 |
| 15 | 18.631 | 4.7587 | 1108 | 832 | 4.9 | 13744 | 4.9 | 0.277 |
| 16 | 19.265 | 4.6034 | 1108 | 951 | 5.7 | 10741 | 3.8 | 0.190 |
| 17 | 19.638 | 4.5168 | 1021 | 507 | 3.0 | 4543 | 1.6 | 0.150 |
| 18 | 20.427 | 4.3441 | 989 | 1637 | 9.7 | 20628 | 7.4 | 0.212 |
| 19 | 21.121 | 4.2030 | 1016 | 606 | 3.6 | 12612 | 4.5 | 0.350 |
| 20 | 21.636 | 4.1042 | 889 | 1755 | 10.4 | 36261 | 13.0 | 0.347 |
| 21 | 22.224 | 3.9968 | 889 | 287 | 1.7 | 1714 | 0.6 | 0.100 |
| 22 | 23.214 | 3.8286 | 899 | 621 | 3.7 | 9548 | 3.4 | 0.258 |
| 23 | 23.568 | 3.7718 | 853 | 658 | 3.9 | 11310 | 4.0 | 0.289 |
| 24 | 23.962 | 3.7106 | 859 | 372 | 2.2 | 8546 | 3.1 | 0.386 |
| 25 | 24.537 | 3.6250 | 895 | 163 | 1.0 | 1222 | 0.4 | 0.126 |
| 26 | 25.246 | 3.5249 | 848 | 407 | 2.4 | 5619 | 2.0 | 0.232 |
| 27 | 25.857 | 3.4428 | 779 | 1484 | 8.8 | 22642 | 8.1 | 0.256 |
| 28 | 26.704 | 3.3355 | 785 | 180 | 1.1 | 1699 | 0.6 | 0.159 |
| 29 | 27.021 | 3.2972 | 737 | 345 | 2.1 | 11718 | 4.2 | 0.571 |
| 30 | 27.301 | 3.2639 | 698 | 521 | 3.1 | 18187 | 6.5 | 0.586 |
| 31 | 27.657 | 3.2228 | 698 | 314 | 1.9 | 4301 | 1.5 | 0.230 |
| 32 | 28.625 | 3.1159 | 648 | 325 | 1.9 | 6310 | 2.3 | 0.326 |
| 33 | 29.020 | 3.0744 | 654 | 188 | 1.1 | 7215 | 2.6 | 0.644 |
| 34 | 29.712 | 3.0044 | 658 | 361 | 2.2 | 4547 | 1.6 | 0.211 |
| 35 | 30.204 | 2.9565 | 622 | 111 | 0.7 | 3010 | 1.1 | 0.454 |
| 36 | 30.793 | 2.9013 | 630 | 220 | 1.3 | 2516 | 0.9 | 0.192 |
| 37 | 31.704 | 2.8200 | 585 | 211 | 1.3 | 3290 | 1.2 | 0.261 |
| 38 | 32.573 | 2.7468 | 571 | 126 | 0.7 | 4095 | 1.5 | 0.546 |
| 39 | 32.985 | 2.7133 | 549 | 155 | 0.9 | 4095 | 1.5 | 0.444 |
| 40 | 34.349 | 2.6087 | 543 | 101 | 0.6 | 6110 | 2.2 | 1.012 |
| 41 | 34.790 | 2.5766 | 566 | 111 | 0.7 | 2264 | 0.8 | 0.344 |
| 42 | 36.129 | 2.4841 | 497 | 80 | 0.5 | 2551 | 0.9 | 0.536 |
| 43 | 38.358 | 2.3447 | 492 | 195 | 1.2 | 4681 | 1.7 | 0.403 |

NOTE:
Intensity = Counts, 2T(0) = 0.0(deg), Wavelength to Compute d-Spacing = 1.54059 Å (Cu/K-alpha1)

### Example 26

(R)—N-(4-(3-aminopiperidin-1-yl)-5-bromo-1H-pyrrolo[2,3-b]pyridin-3-yl)cyclopropanecarboxamide di-ethane sulfonic acid salt: (R)—N-(4-(3-aminopiperidin-1-yl)-5-bromo-1H-pyrrolo[2,3-b]pyridin-3-yl)cyclopropanecarboxamide di-ethanesulfonic acid salt hydrate was heated to 130° C. in a DSC apparatus and then cooled to room temperature. The anhydrate re-absorbs water at 80% RH and is converted to anhydrate. The physical properties of the non-solvated di-esylate salt of (R)—N-(4-(3-aminopiperidin-1-yl)-5-bromo-1H-pyrrolo[2,3-b]pyridin-3-yl)cyclopropanecarboxamide are summarized in Table 50 below.

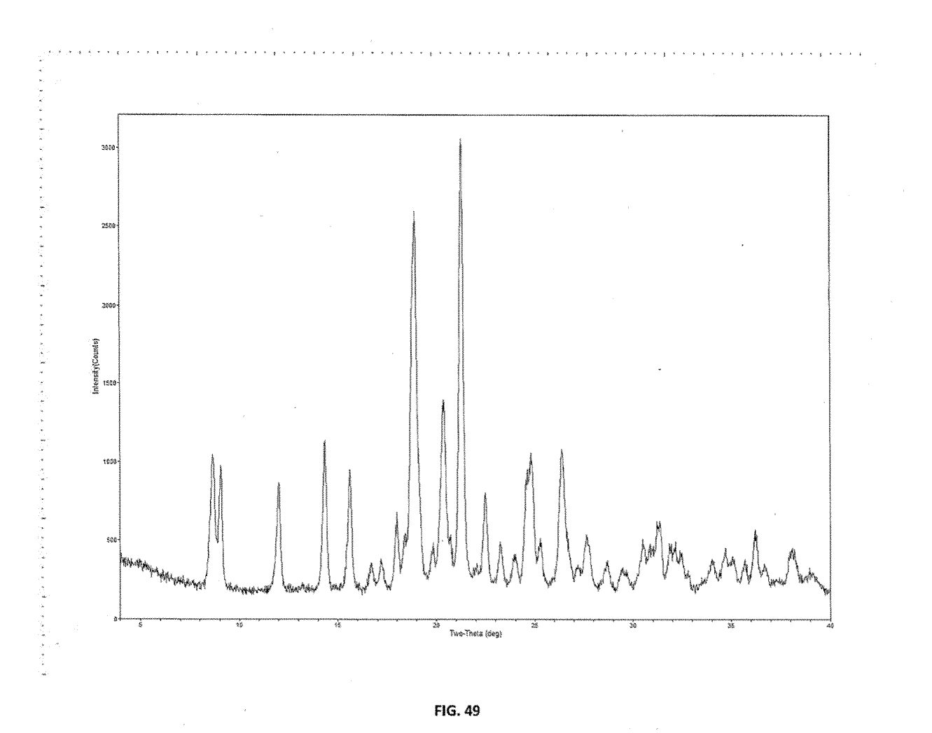

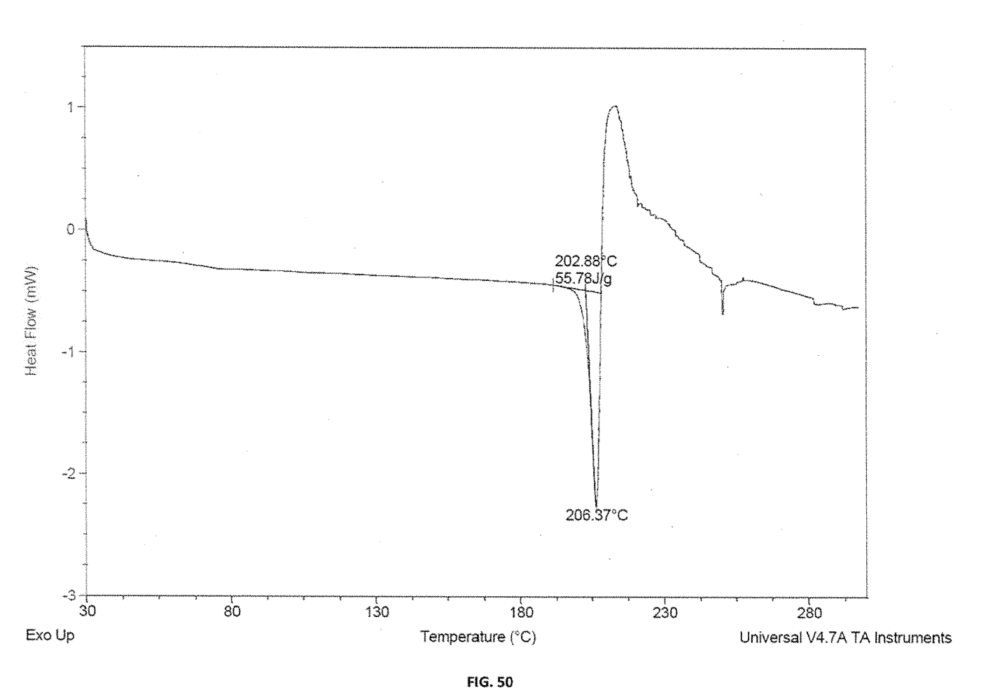

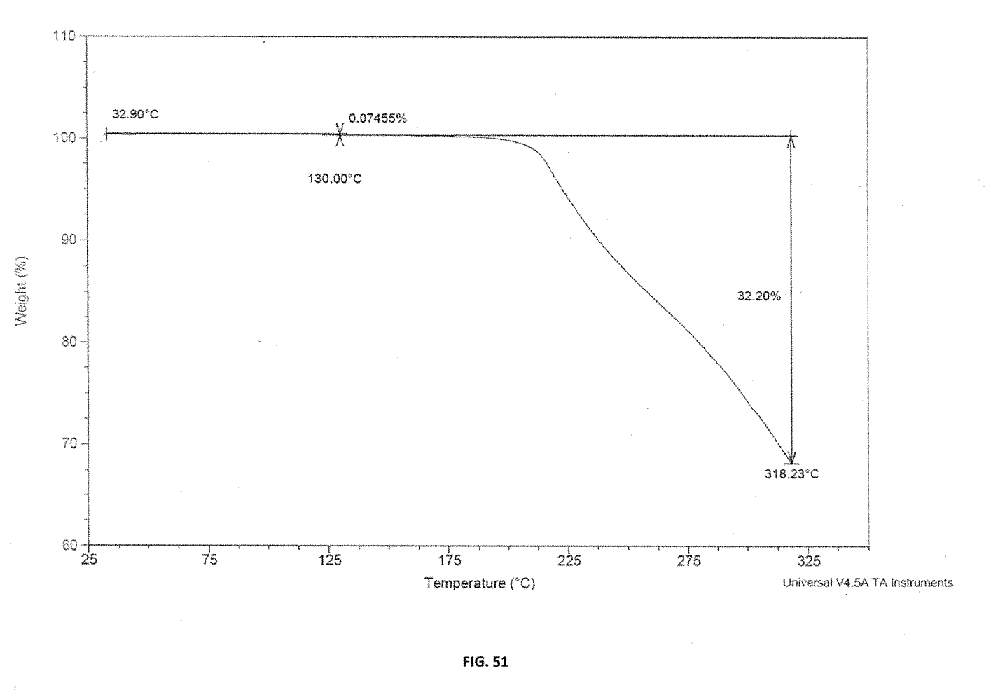

TABLE 50

| | |
|---|---|
| XRPD | FIG. 49. |
| DSC | FIG. 50: Endotherm with extrapolated onset temperature |
| | about 203° C. and enthalpy of fusion about 56 J/g. |
| TGA | FIG. 51: Less than 0.08% weight loss up to about 130° C. |

### Example 27

Maleic acid salt of (R)—N-(4-(3-aminopiperidin-1-yl)-5-bromo-1H-pyrrolo[2,3-b]pyridin-3-yl)cyclopropanecarboxamide: Prepared by methods similar to those disclosed herein. The physical properties of the non-solvated mono-maleate salt of (R)—N-(4-(3-aminopiperidin-1-yl)-5-bromo-1H-pyrrolo[2,3-b]pyridin-3-yl)cyclopropanecarboxamide are summarized in Table 51 below.

TABLE 51

| | |
|---|---|
| Aqueous | >10 at pH ≤ 7 |
| Solubility | |
| (mg/mL) | |
| DSC/TGA | Melting point about 214° C. No weight loss until |
| | melt/degradation. |
| DVS | Non-hygroscopic, 0.3% wt./wt. gain at 80% RH. |

### Example 28

(R)—N-(4-(3-aminopiperidin-1-yl)-5-bromo-1H-pyrrolo[2,3-b]pyridin-3-yl)cyclopropanecarboxamide HBr salt methanol solvate: Prepared by methods similar to those disclosed herein. The physical properties of the methanol solvate of the HBr salt of (R)—N-(4-(3-aminopiperidin-1-yl)-5-bromo-1H-pyrrolo[2,3-b]pyridin-3-yl)cyclopropanecarboxamide are summarized in Table 52 below.

TABLE 52

| | |
|---|---|
| DSC/TGA | Onset of desolvation at approximately 112° C. |

### Example 29

(R)—N-(4-(3-aminopiperidin-1-yl)-5-bromo-1H-pyrrolo[2,3-b]pyridin-3-yl)cyclopropanecarboxamide di-HCl salt: Prepared by methods similar to those disclosed herein. The physical properties of the non-solvated di-HCl salt of (R)—N-(4-(3-aminopiperidin-1-yl)-5-bromo-1H-pyrrolo[2,3-b]pyridin-3-yl)cyclopropanecarboxamide are summarized in Table 53 below.

TABLE 53

| | |
|---|---|
| DSC/TGA | Single endotherm due to melting/decomposition at ~209° |
| | C. ~7% weight loss by TGA, which occurs upon melting. |
| DVS | Non-hygroscopic below 80% RH. Converts to a |
| | crystalline dihydrate form above 80% RH. |

### Example 30

(R)—N-(4-(3-aminopiperidin-1-yl)-5-bromo-1H-pyrrolo[2,3-b]pyridin-3-yl)cyclopropanecarboxamide di-HBr salt: Prepared by methods similar to those disclosed herein. The physical properties of the non-solvated di-HBr salt of (R)—N-(4-(3-aminopiperidin-1-yl)-5-bromo-1H-pyrrolo[2,3-b]pyridin-3-yl)cyclopropanecarboxamide are summarized in Table 54 below.

TABLE 54

| | |
|---|---|
| DSC/TGA | Anhydrous solids with a concurrent melt/decomposition |
| | exotherm at ~273° C. Analysis of the post-DVS solids |
| | shows ~4% step-wise weight loss by TGA with an onset of |
| | desolvation at 81.2° C. Further heating on the DSC shows |
| | the same exothermic behavior as the starting material |
| | however, the onset of the exotherm is at ~217° C. |
| DVS | Non-hygroscopic below 80% RH. Upon desorption the solids |
| | were found to convert to a new mono-hydrate crystal form. |

### Example 31

Crystalline forms of (R)—N-(4-(3-aminopiperidin-1-yl)-5-bromo-1H-pyrrolo[2,3-b]pyridin-3-yl)cyclopropanecarboxamide salts with tartaric, succinic, and phosphoric acids, and solvates thereof, were also prepared by methods similar to those disclosed herein (data not shown).

### Example 32

Excipient compatibility study of (R)—N-(4-(3-aminopiperidin-1-yl)-5-bromo-1H-pyrrolo[2,3-b]pyridin-3-yl)cyclopropanecarboxamide mono-ethanesulfonic acid salt: (R)—N-(4-(3-aminopiperidin-1-yl)-5-bromo-1H-pyrrolo[2,3-b]pyridin-3-yl)cyclopropanecarboxamide mono-ethanesulfonic acid salt and excipients were weighed according to formulation of Composite 2 and Composite 4 with equivalent incremental addition and ground thoroughly in a mortar, respectively (Table 55). Composite blanks (same formulation without API) were placed as controls with the same process. Each of about 100 mg of composite blends was pressed to a tablet formulation (diameter 8 mm, thickness 1 mm) with manual sheeter under the pressure (4 MPa) for half a minute. The tablet was transferred into a 40 mL glass vial for the storage conditions (Table 56). The stability samples in the vials were capped and sealed with parafilm. The samples were placed at 30° C. (closed), 40° C. (closed) and 50° C. (closed) for 0 day, 1 month, 3 months, 6 months, 12 months and backup in duplicate and monitored with physical appearance, impurities/degradants and recovery at each time point, respectively. The excipient blanks as controls were conducted in single.

TABLE 55

| | | | Actual weight (mg) for | |
|---|---|---|---|---|
| | Composition (%, w/w) | | tablets | |
| | Composite- | Composite- | Composite- | Composite- |
| Excipients | 2 blend | 4 blend | 2 blend | 4 blend |
| Mono-esylate | 5 | 5 | 202.60 | 199.54 |
| MCC | 44.5 | 68 | 1782.31 | 2731.29 |
| Starch | 44.5 | 20 | 1780.08 | 798.85 |
| Crocarmellose | 5 | — | 200.95 | — |
| sodium | | | | |
| Crospovidone | — | 5 | — | 200.66 |
| Silicon dioxide | — | 1 | — | 42.29 |
| Mg stearate | 1 | 1 | 40.14 | 40.26 |

TABLE 56

| | | Recovery (%) | | | | | TRS (%) | | | | |
|---|---|---|---|---|---|---|---|---|---|---|---|
| | Stress | | 1^{st} | 3^{rd} | 6^{th} | 12^{th} | | 1^{st} | 3^{rd} | 6^{th} | 12^{th} |
| Samples | condition | Initial | month | month | month | month | Initial | month | month | month | month |
| Mono-esylate | 50° C.- | 96.1 | 95.2 | 95.0 | 95.1 | | 0.41 | 0.49 | 0.56 | 0.51 | |
| composite-2 | closed | | | | | | | | | | |
| blend (MCC, | 40° C.- | | 95.1 | 94.3 | 95.3 | | | 0.40 | 0.43 | 0.41 | |
| Starch, etc) | closed | | | | | | | | | | |
| | 30° C.- | | 95.7 | 95.3 | 94.6 | | | 0.42 | 0.40 | 0.33 | |
| | closed | | | | | | | | | | |
| Mono-esylate | 50° C.- | 97.4 | 95.1 | 95.8 | 96.4 | | 0.41 | 0.56 | 0.56 | 0.48 | |
| composite-4 | closed | | | | | | | | | | |
| blend (MCC, | 40° C.- | | 96.6 | 97.3 | 97.7 | | | 0.51 | 0.42 | 0.39 | |
| Starch, | closed | | | | | | | | | | |
| Crospovidone | 30° C.- | | 96.8 | 96.3 | 97.0 | | | 0.44 | 0.43 | 0.39 | |
| etc) | closed | | | | | | | | | | |

### Example 33

Chemical characteristics of (R)—N-(4-(3-aminopiperidin-1-yl)-5-bromo-1H-pyrrolo[2,3-b]pyridin-3-yl)cyclopropanecarboxamide free base (Form A) and maleate salt crystal forms: The stability of the crystal forms described in Examples 4 and 27 was measured neat as well as in a Powder-in-Capsule (PiC) formulation. The stability as neat powder was measured over a period of 1-year for the free base and 3-months for the maleate salt, under ambient (25° C./60% RH) and accelerated (40° C./75% RH) conditions. The free base was found to show very good stability with no chiral or achiral degradation observed under any condition for a period of 1-year. The maleate salt under the same conditions, after 3-months on stability, was found to show very slight degradation under accelerated conditions. It should be noted that the maleate salt used in the neat powder stability study contained high levels (˜9% wt./wt.) of residual tetrahydrofuran (THF). The PiC stability using 15 mg of (R)—N-(4-(3-aminopiperidin-1-yl)-5-bromo-1H-pyrrolo[2,3-b]pyridin-3-yl)cyclopropanecarboxamide in Size 0 hard gelatin capsules was carried out over a period of 2-months at 5° C., ambient and accelerated conditions. Similar to the neat powder, the PiC formulation did not cause any significant physical or chemical degradation in the free base formulation and the maleate salt, containing no residual THF, was also found to be stable.

To further investigate the chemical properties of these crystal forms, an abbreviated excipient compatibility study was run at 50° C. with 20% H₂O added. The excipients used in the study consisted of common diluents and disintegrants and the study was conducted over a period of 4 weeks. The free base was found to show significant degradation with lactose and Ac-Di-Sol® (croscarmellose sodium) whereas the maleate salt was found to show significant degradation with all excipients investigated. Notably, the maleate salt also showed the same degradant formation as the free base in lactose suggesting that the salt does not fully attenuate the presumed Maillard reaction. Overall, the free base Form A appears to be more stable with excipients than the maleate salt of (R)—N-(4-(3-aminopiperidin-1-yl)-5-bromo-1H-pyrrolo[2,3-b]pyridin-3-yl)cyclopropanecarboxamide.

### Example 34

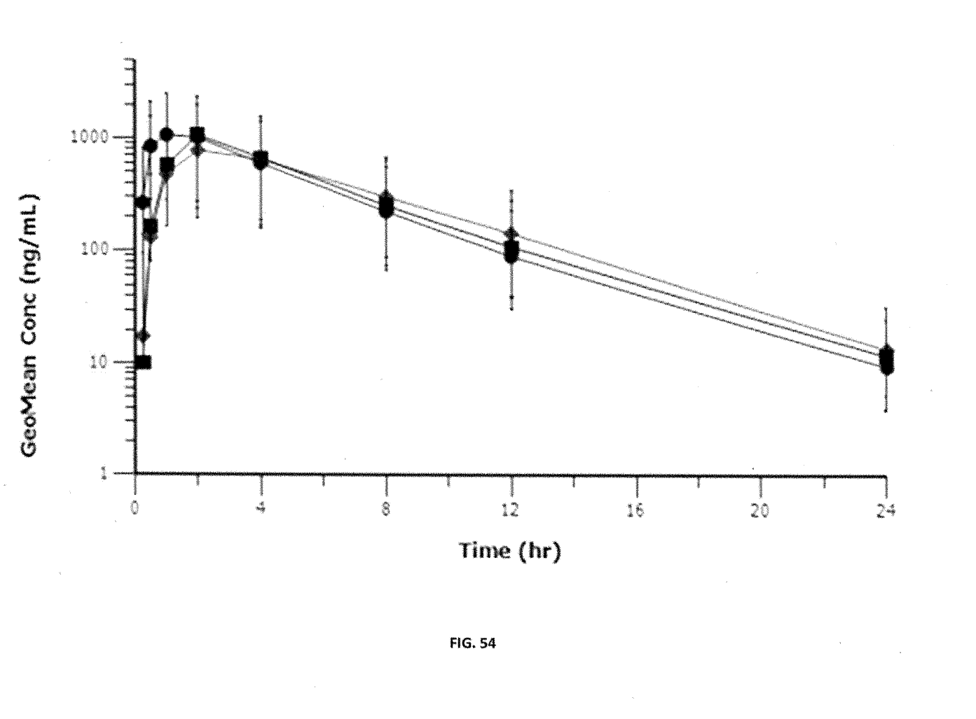

Biological characteristics of (R)—N-(4-(3-aminopiperidin-1-yl)-5-bromo-1H-pyrrolo[2,3-b]pyridin-3-yl)cyclopropanecarboxamide free base (Form A) and maleate salt crystal forms: The oral absorption of the crystal forms described in Examples 4 and 27 as PiC formulations was investigated in a preclinical pharmacokinetic study conducted in canines using a reduced Latin square crossover design at 3 mg/kg to compare the solid formulations to a solution of (R)—N-(4-(3-aminopiperidin-1-yl)-5-bromo-1H-pyrrolo[2,3-b]pyridin-3-yl)cyclopropanecarboxamide. The results of this study showed that the area under the curve (AUC) variability is consistent between (R)—N-(4-(3-aminopiperidin-1-yl)-5-bromo-1H-pyrrolo[2,3-b]pyridin-3-yl)cyclopropanecarboxamide formulations (PiC vs. solution) and that the maximal concentration (Cₘₐₓ) data suggest the maleate salt PiC is less variable, however high variability was observed throughout the study. Consistent terminal elimination phases as well as half-life (t_{1/2}) values were observed between formulations and the time to maximum concentration (Tₘₐₓ) values were consistent with the toxicokinetic values obtained in GLP toxicology studies. No trends observed by AUC or t_{1/2} could be discerned for each of the three formulations. In addition, the median Cₘₐₓ values were similar across all formulations. (See FIG. 54). Overall, no significant differences in absorption were found between the freebase and maleate crystal forms of (R)—N-(4-(3-aminopiperidin-1-yl)-5-bromo-1H-pyrrolo[2,3-b]pyridin-3-yl)cyclopropanecarboxamide.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-24. (canceled)

25. A pharmaceutical formulation comprising a DNA damaging agent and a non-solvated crystalline form of the free base of (R)—N-(4-(3-aminopiperidin-1-yl)-5-bromo-1H-pyrrolo[2,3-b]pyridin-3-yl)cyclopropanecarboxamide.

26. (canceled)

27. The formulation of claim 25, wherein the DNA damaging agent is selected from: gemcitabine, irinotecan, temozolomide, capecitabine, camptothecin, cisplatin, ara-C, and 5-FU.

28. The formulation of claim 25, further comprising an excipient.

29. The formulation of claim 28, wherein the formulation is a tablet for oral delivery.

30. A method of treating a disease or disorder modulated by CHK1, comprising administering the formulation of claim 25, to a patient in need thereof.

31. The method of claim 30, wherein the disease is cancer.

32-45. (canceled)
